(19) United States
(12) Patent Application Publication
(10) Pub. No.: US 2025/0270507 A1
(43) Pub. Date: Aug. 28, 2025

(54) METHODS FOR PRODUCING CARTILAGE AND BONES

(71) Applicant: Murdoch Children's Research Institute, Parkville, Victoria (AU)

(72) Inventors: John Bateman, Parkville, Victoria (AU); Shireen Lamandé, Parkville, Victoria (AU); Elizabeth Ng, Parkville, Victoria (AU)

(21) Appl. No.: 18/698,337

(22) PCT Filed: Oct. 14, 2022
(86) PCT No.: PCT/AU2022/051242

§ 371 Date: Apr. 3, 2024

(30) Foreign Application Priority Data
Oct. 14, 2021 (AU) 2021903310

(51) Int. Cl. C12N 5/077; A61K 35/32; G01N 33/50

(52) U.S. Cl. CPC C12N 5/0655; A61K 35/32; C12N 5/0654; G01N 33/5044; C12N 2501/115; C12N 2501/15; C12N 2501/155; C12N 2501/415; C12N 2506/45; C12N 2513/00

(57) ABSTRACT

The present application relates to methods for inducing the differentiation of stem cells into chondrocyte progenitors and chondrocytes in vitro. The methods also relate to the production of in vitro-engineered cartilage and bone and related biomaterials as well as methods of drug-screening and modeling the bone- and cartilage-related diseases and disorders.

### FIELD

The present application relates to methods for inducing the differentiation of stem cells into chondrocyte progenitors and chondrocytes in vitro. The methods also relate to the production of in vitro-engineered cartilage and bone and related biomaterials as well as methods of drug-screening and modelling of bone- and cartilage-related diseases and disorders.

### BACKGROUND

The major connective tissues of the body, such as skin, tendon, ligaments, cartilage and bone provide the crucial structural and informational framework necessary for development. The extensive extracellular matrix (ECM) of connective tissues is a complex interacting network of proteins, glycoproteins and proteoglycans providing the dynamic and essential three-dimensional environment that supports the maintenance, growth and differentiation of cells. In addition to providing a highly organized framework, the ECM mediates signals to and from cells involved in important biological processes such as cell differentiation and migration during development and repair processes.

Chondrocytes at the developing joint articulating surfaces form a distinct population of articular chondrocytes which produce the permanent articular cartilages. These articular chondrocytes synthesize a distinct cartilage extracellular matrix with the specialized properties required to provide resilience to the repetitive mechanical loading that occurs during diarthrodial joint function. These permanent articular chondrocytes produce the characteristic extracellular components of cartilage, such as the major structural collagen II and aggrecan, they have a specific repertoire of gene expression and stoichiometry of cartilage extracellular matrix components that supports their crucial role in joint homeostasis and function. Because of these important biological properties, it is necessary to be able to provide biomaterials, suitable for use in tissue repair/regeneration or disease modelling, which have tissue-specific ECM and which are phenotypically accurate.

Embryonic chondrocytes can also follow a developmental pathway that forms the transient chondrocytes of the cartilage growth plate. The chondrocytes entering this pathway produce the highly organized growth plate structures that are the crucial driver of longitudinal bone growth by sequential maturation, hypertrophy and transition to bone.

Despite advances in orthopedic surgery, finding effective treatments for musculoskeletal injuries, cartilage and meniscus injuries, inherited diseases of cartilage and bone development and homeostasis, and other joint related diseases and disorders, such as osteoarthritis, remain a significant clinical challenge. Additionally, the complex orchestration of cartilage and endochondral bone formation provides a fertile landscape for human inherited disorders with abnormal cartilage development and bone growth (osteochondrodysplasias). The current genetic skeletal disorders nosology includes more than 450 disorders reflecting their diverse manifestations. While recent advances in genomic technologies have revealed many underlying gene mutations, understanding the cellular disease mechanisms lags, and few well-targeted therapies are available. In addition to genetic skeletal disease, acquired cartilage disorders, such as osteoarthritis, are a huge clinical problem. The central pathological feature of osteoarthritis is the progressive destruction of articular cartilage. Articular cartilage has a poor reparative capacity and ultimately it is the erosion of cartilage that signifies end-stage disease. An approach to cartilage regeneration involves repair of the cartilage with autologous or allogenic neo-cartilage constructs. This is a rapidly evolving field, however there is an unmet need as optimal conditions to robustly produce cartilage for therapeutic use are yet to be determined. Human iPSC-derived cartilage cells, where developmental stages and extracellular matrix composition can be precisely controlled, offer an important tool to produce either neo-cartilage (containing cells) or decellularized cartilage as biomaterial scaffold, for cartilage regeneration. A controlled in vitro method would enable the provision of enhanced biomaterials with more desirable biological and biomechanical properties. In addition, an in vitro disease model system where the process of endochondral cartilage maturation and bone formation are faithfully recapitulated is needed to provide a better understanding of the molecular pathology of disease and to enable the identification of new therapeutic options.

Through detailed studies the inventors have developed a pluripotent stem cell differentiation protocol that recapitulates paraxial mesoderm development in vitro to produce sclerotome, chondrocyte progenitors then chondrocytes that may be induced to differentiate into articular chondrocytes, or alternatively, chondrocytes that mature to hypertrophic chondrocytes and can transition to osteoblasts and produce a mineralized extracellular matrix. The protocols described herein recapitulate the key steps in growth plate development and endochondral ossification in vitro and thereby provide biomaterials that have tissue-specific ECM and which are phenotypically accurate. The methods and biomaterials described herein provide a model system for interrogating the mechanisms of diseases, disorders and conditions that affect cartilage and endochondral bone formation, and for drug screening in diseases, disorders and conditions that that affect cartilage and endochondral bone formation.

### SUMMARY OF INVENTION

According to a first aspect the present invention provides a method for generating chondrocytes, chondrocyte-like cells, cartilage-like tissue, cartilage, or a combination thereof, the method comprising:
a) culturing a three-dimensional aggregation of sclerotome cells with an FGF pathway activator to produce a three-dimensional aggregation of chondrocyte progenitor cells; and
b) culturing the three-dimensional aggregation of chondrocyte progenitor cells generated in step a) in the absence of said FGF pathway activator to produce a three-dimensional aggregation of chondrocytes, or chondrocyte-like cells, that express COL2A1 and ACAN;
wherein the method comprises transferring the three-dimensional aggregation of sclerotome cells or the three-dimensional aggregation of chondrocyte progenitor cells to orbital culture.

In an embodiment of the first aspect the method further comprises culturing said three-dimensional aggregation of chondrocytes, or chondrocyte-like progenitor cells with a triiodothyronine (T3) to produce a population of hypertrophic chondrocytes or a hypertrophic chondrocyte-like cells, hypertrophic cartilage-like tissue, hypertrophic cartilage, or a combination thereof that express COL10A1.

In another embodiment of the first aspect, the method further comprises culturing said three-dimensional aggregation of chondrocytes, or chondrocyte-like cells with TGF-beta3, preferably for a period of at least 2 weeks to about 4 weeks, to produce a population of articular chondrocytes or articular chondrocyte-like cells, articular cartilage-like tissue, articular cartilage, or a combination thereof. In some embodiments said three-dimensional aggregation of chondrocytes, or chondrocyte-like cells are cultured with TGF-beta3 to produce a population of articular chondrocytes or articular chondrocyte-like cells, articular cartilage-like tissue, articular cartilage, or a combination thereof that express PRG4.

In a second aspect, the invention provides a method for generating articular (non-hypertrophic) chondrocytes or articular chondrocyte-like cells, articular cartilage-like tissue, articular cartilage, or a combination thereof, the method comprising:
a) culturing a three-dimensional aggregation of sclerotome cells with an FGF pathway activator to produce a three-dimensional aggregation of chondrocyte progenitor cells;
b) culturing the three-dimensional aggregation of chondrocyte progenitor cells with an FGF pathway activator and a TGF-beta agonist to produce a three-dimensional aggregation of chondrocytes, or chondrocyte-like cells, that express COL2A1 and ACAN;
c) culturing said three-dimensional aggregation of chondrocytes, or chondrocyte-like cells with a TGF-beta agonist for an extended period of time to produce articular (non-hypertrophic) chondrocytes or articular chondrocyte-like cells, articular cartilage-like tissue, articular cartilage, or a combination thereof
wherein the method comprises transferring the three-dimensional aggregation of sclerotome cells, the three-dimensional aggregation of chondrocyte progenitor cells, or the three-dimensional aggregation of chondrocytes, or chondrocyte-like cells to orbital culture.

In a third aspect, the invention provides a method of producing a population of osteoblasts or a bone-like organoid, the method comprising:
a) deriving hypertrophic chondrocytes, hypertrophic chondrocyte-like cells, cartilage-like tissue, cartilage, according to the method of the first aspect; and
b) culturing in orbital culture the hypertrophic chondrocytes, hypertrophic chondrocyte-like cells, cartilage-like tissue, cartilage with an osteogenic culture medium to generate osteoblasts or a bone-like organoid that express COL1A1 and COL1A2.

In a fourth aspect, the present invention provides chondrocytes, chondrocyte-like cells, cartilage-like tissue, cartilage, or a combination thereof produced according to the method of the first aspect.

In a fifth aspect, the present invention provides articular (non-hypertrophic) chondrocytes or articular chondrocyte-like cells, articular cartilage-like tissue, articular cartilage, or a combination thereof produced according to the method of the second aspect.

In a sixth aspect, the present invention provides osteoblasts or bone-like organoid produced according to the method of the third aspect.

In a seventh aspect, the present invention provides a decellularized scaffold prepared by decellularizing the three-dimensional aggregation of chondrocytes, or chondrocyte-like cells, cartilage-like tissue or cartilage or combination thereof of the fourth aspect, articular (non-hypertrophic) chondrocytes or articular chondrocyte-like cells, articular cartilage-like tissue, articular cartilage, or a combination thereof of the fifth aspect or the osteoblasts or bone-like organoid of the sixth aspect to produce a decellularized scaffold.

In a seventh aspect, the present invention provides a composition comprising a homogenous population of iPSC-derived chondrocytes, wherein said cells express collagen 2 (COL2A1) and ACAN at a ratio in the range of 20:1 to 5:1, preferably about 10:1 and does not substantially express COL10A1.

In an eighth aspect, the present invention provides a composition comprising a homogenous population of iPSC-derived hypertrophic chondrocytes, wherein said cells express expresses collagen 2 (COL2A1) and COL10A1 at a ratio in the range of 1:1 to about 2.5:1.

In a ninth aspect, the present invention provides a method of treating a chondral defect or an osteochondral defect in a subject in need thereof comprising a) deriving chondrocytes, chondrocyte-like cells, cartilage-like tissue, cartilage, or a combination thereof, according to the first aspect, articular (non-hypertrophic) chondrocytes or articular chondrocyte-like cells, articular cartilage-like tissue, articular cartilage, or a combination thereof of the second aspect or osteoblasts or bone-like organoid according to the third aspect, and b) administering chondrocytes, chondrocyte-like cells, cartilage-like tissue, cartilage, or combination thereof or osteoblasts or bone-like organoid to the subject.

In a tenth aspect, the present invention provides a method of testing candidate chondrogenic or osteogenic modulating substances, the method comprising:
a) carrying out the method of any one of the first, second or third aspects, wherein said test substance is included in any one, or more, of the culture steps of the method;
b) assessing the effect of the test substance on chondrocyte or osteoblast proliferation, maintenance and/or differentiation compared to a control population generated in the absence of test substance; and
c) identifying the test substance as a candidate chondrogenic or osteogenic modulating substance if the test substance increases or decreases proliferation, and/or affects chondrocyte or osteoblast maintenance or differentiation compared to the control.

In an eleventh aspect, the present invention provides use of chondrocytes, chondrocyte-like cells, cartilage-like tissue, cartilage, or a combination thereof of the first aspect, articular (non-hypertrophic) chondrocytes or articular chondrocyte-like cells, articular cartilage-like tissue, articular cartilage, or a combination thereof of the second aspect or osteoblasts or bone-like organoid according to the third aspect, for disease modelling or testing therapeutic agents for a bone or cartilage disease or disorder.

Numbered statements of the invention are as follows:
1. A method for generating chondrocytes, chondrocyte-like cells, cartilage-like tissue, cartilage, or a combination thereof, the method comprising:
   a) culturing a three-dimensional aggregation of sclerotome cells with an FGF pathway activator to produce a three-dimensional aggregation of chondrocyte progenitor cells; and
   b) culturing the three-dimensional aggregation of chondrocyte progenitor cells generated in step a) in the absence of said FGF pathway activator to produce a three-dimensional aggregation of chondrocytes, or chondrocyte-like cells, that express COL2A1 and ACAN;
wherein the method comprises transferring the three-dimensional aggregation of sclerotome cells or the three-dimensional aggregation of chondrocyte progenitor cells to orbital culture.
2. The method of form 1, wherein said sclerotome cells are obtained by a method comprising:
   i) culturing a population of human pluripotent progenitor cells with a composition comprising a TGF-beta pathway activator, a Wnt pathway activator, an FGF pathway activator, and a P13K inhibitor for a time period of about 24 hours;
   ii) culturing the cells of step i) with a composition comprising a TGF-beta pathway inhibitor, a Wnt pathway activator, an FGF pathway activator, and a BMP pathway inhibitor for a time period of about 24 hours;
   iii) culturing the cells of step ii) with a composition comprising a Wnt pathway inhibitor, a BMP pathway inhibitor and a MEK/ERK pathway inhibitor for a time period of about 24 hours;
   iv) contacting the cells of step iii) with a composition comprising a Wnt pathway inhibitor and a Hedgehog pathway activator, for a time period of about 72 hours to generate sclerotome cells.
3. The method of form 1 or 2, wherein said sclerotome cells express any one of PAX1, SOX9, FOXC2, PAX9, NKX3.2/BAPX1 and TWIST1,
4. The method of any one of forms 1-4, wherein said sclerotome cells are derived from a human embryonic stem cell population (hESC) or a human induced pluripotent stem cell population (iPSC).
5. The method of form 4 wherein said iPSCs are derived from feeder-free cell culture.
6. The method of any one of forms 1-5, wherein step a) comprises transferring said aggregation of sclerotome cells to orbital culture at any time from the commencement of step a) onwards.
7. The method of any one of forms 1-6, wherein the three-dimensional aggregation of chondrocyte cells express one or more further genes selected from the group consisting of COL11A1, COL11A2, COL9A1, COL9A2, COL9A3, MATN1 and MATN3.
8. The method of any one of forms 1-7, wherein said chondrocytes, chondrocyte-like cells, cartilage-like tissue, cartilage, or a combination thereof expresses collagen 2 (COL2A1) and ACAN at a ratio in the range of from 20:1 to 5:1, preferably about 10:1 and does not substantially express COL10A1.
9. The method of any one of forms 1-8, wherein said chondrocytes, chondrocyte-like cells, cartilage-like tissue, cartilage, or a combination thereof does not express collagen COL1A1 or COL1A2 in levels greater than about 0.2% of the level of COL2A1.
10. The method of any one of forms 1-9, wherein the method further comprises culturing said three-dimensional aggregation of chondrocytes, or chondrocyte-like cells with triiodothyronine (T3) to produce a population of hypertrophic chondrocytes or a hypertrophic chondrocyte-like cells, hypertrophic cartilage-like tissue, hypertrophic cartilage, or a combination thereof that express COL10A1.
11. The method of any one of forms 1-10, wherein the method comprises transferring said aggregation of sclerotome cells to orbital culture at any time from day 7 of step a) onwards.
12. The method of form 10 or 11, wherein said hypertrophic chondrocytes, hypertrophic chondrocyte-like cells, hypertrophic cartilage-like tissue, hypertrophic cartilage, or a combination thereof expresses COL2A1 and COL10A1.
13. The method of form 12, wherein said hypertrophic chondrocytes, hypertrophic chondrocyte-like cells, hypertrophic cartilage-like tissue, hypertrophic cartilage, or a combination thereof expresses COL2A1 and COL10A1 at a ratio in the range of about 1:1 to about 2.5:1.
14. The method of any one of forms 1-9, wherein the method further comprises culturing said three-dimensional aggregation of chondrocytes, or chondrocyte-like cells with TGF-beta3, preferably for a period of at least 2 weeks to about 7 weeks, to produce a population of articular chondrocytes or articular chondrocyte-like cells, articular cartilage-like tissue, articular cartilage, or a combination thereof that express PRG4.
15. A method for generating articular (non-hypertrophic) chondrocytes or articular chondrocyte-like cells, articular cartilage-like tissue, articular cartilage, or a combination thereof, the method comprising:
   a) culturing a three-dimensional aggregation of sclerotome cells with an FGF pathway activator to produce a three-dimensional aggregation of chondrocyte progenitor cells;
   b) culturing the three-dimensional aggregation of chondrocyte progenitor cells with an FGF pathway activator and a TGF-beta agonist to produce a three-dimensional aggregation of chondrocytes, or chondrocyte-like cells, that express COL2A1 and ACAN;
   c) culturing said three-dimensional aggregation of chondrocytes, or chondrocyte-like cells with a TGF-beta agonist for an extended period of time to produce articular (non-hypertrophic) chondrocytes or articular chondrocyte-like cells, articular cartilage-like tissue, articular cartilage, or a combination thereof
   wherein the method comprises transferring the three-dimensional aggregation of sclerotome cells, the three-dimensional aggregation of chondrocyte progenitor cells, or the three-dimensional aggregation of chondrocytes, or chondrocyte-like cells to orbital culture.
16. The method of form 14 or 15, wherein said articular chondrocytes or articular chondrocyte-like cells, articular cartilage-like tissue, articular cartilage, or a combination thereof expresses COL2A1, ACAN, PRG4, and optionally, one or more further genes selected from the group consisting of ASPN, CILP and CILP2.
17. The method of any one of forms 1-16, wherein said culturing in step a) is for a period of at least 7 days.
18. The method of any one of forms 1-16, wherein said culturing in step a) is for a period of from 7-21 days.
19. The method of any one of forms 1-16, wherein said culturing in step a) is for a period of from about 10-17 days.
20. The method of any one of forms 1-16, wherein said culturing in step a) is for a period of about 14 days.
21. The method of form 15 or 16, wherein said culturing in step a) is for a period of about 7 days.
22. The method of any one of forms 1-21, wherein said culturing in step b) is for a period of at least 7 days.
23. The method of any one of forms 1-21, wherein said culturing in step b) is for a period of from 7-21 days.
24. The method of any one of forms 1-21, wherein said culturing in step b) is for a period of from about 10-17 days.
25. The method of any one of forms 1-21, wherein said culturing in step b) is for a period of about 14 days.
26. The method of any one of forms 1-21 wherein said culturing in step b) is for a period of about 7 days.
27. The method of any one of forms 15 or 16, or 17-25 when dependent on form 15 or 16, wherein said culturing in step c) is for a period of about 2 to about 5 weeks.
28. The method of any one of forms 1 to 27, wherein the FGF pathway activator is selected from the group consisting of FGF2, FGF4, FGF9, FGF19, FGF21, FGF3, FGF5, FGF6, FGF8a, FGF16, FGF17, FGF18, FGF20 and FGF23.
29. The method of form 28, wherein the FGF pathway activator is FGF2.
30. The method of any one of forms 2 to 29, wherein the TGF-beta pathway activator is selected from the group consisting of Activin A, TGF-beta1, TGF-beta2, TGF-beta3, IDE1/2 (IDE1 (1-[2-[(2-Carboxyphenyl)methylene]hydrazide]heptanoic acid), IDE2 (Heptanedioic acid-1-(2-cyclopentylidenehydrazide)), and Nodal.
31. The method of form 30, wherein the TGF-beta pathway activator is Activin A.
32. The method of any one of forms 2 to 31, wherein the Wnt pathway activator is selected from the group consisting of CHIR99021 (6-[[2-[[4-(2,4-Dichlorophenyl)-5-(5-methyl-1H-imidazol-2-yl)-2-pyrimidinyl]amino]ethyl]amino]-3-pyridinecarbonitrile), Wnt-1, Wnt-2, Wnt-2b, Wnt-3a, Wnt-4, Wnt-5a, Wnt-5b, Wnt-6, Wnt-7a, Wnt-7a/b, Wnt-7b, Wnt-8a, Wnt-8b, Wnt-9a, Wnt-9b, Wnt-10a, Wnt-10b, Wnt-11, Wnt-16b, RSPO co-agonists, lithium chloride, TDZD8 (4-Benzyl-2-methyl-1,2,4-thiadiazolidine-3,5-dione), BIO-Acetoxime ((2′Z,3′E)-6-Bromoindirubin-3′-acetoxime), A1070722 (1-(7-Methoxyquinolin-4-yl)-3-[6-(trifluoromethyl)pyridin-2-yl]urea), HLY78 (4-Ethyl-5,6-Dihydro-5-methyl-[1,3]dioxolo[4,5-j]phenanthridine), CID 11210285 hydrochloride (2-Amino-4-(3,4-(methylenedioxy)benzylamino)-6-(3-methoxyphenyl)pyrimidine hydrochloride), WAY-316606, (hetero) arylpyrimidines, IQ1, QS11, SB-216763, and DCA.
33. The method of form 32, wherein the Wnt pathway activator is CHIR99021.
34. The method of any one of forms 2 to 33, wherein the PI3K pathway inhibitor is selected from the group consisting of AS 252424 (5-[[5-(4-Fluoro-2-hydroxyphenyl)-2-furanyl]methylene]-2,4-thiazolidinedione), AS 605240 (5-(6-Quinoxalinylmethylene)-2,4-thiazolidine-2,4-dione), AZD 6482 ((−)-2-[[(1R)-1-[7-Methyl-2-(4-morpholinyl)-4-oxo-4H-pyrido[1,2-a]pyrimidin-9-yl]ethyl]amino]benzoic acid), BAG 956 (α,α,-Dimethyl-4-[2-methyl-8-[2-(3-pyridinyl) ethynyl]-1H-imidazo[4,5-c]quinolin-1-yl]-benzeneacetonitrile), CZC 24832 (5-(2-Amino-8-fluoro[1,2,4]triazolo[1,5-a]pyridin-6-yl)-N-(1,1-dimethylethyl)-3-pyridinesulfonamide), GSK 1059615 (5-[[4-(4-Pyridinyl)-6-quinolinyl]methylene]-2,4-thiazolidenedione), KU 0060648 (4-Ethyl-N-[4-[2-(4-morpholinyl)-4-oxo-4H-1-benzopyran-8-yl]-1-dibenzothienyl]-1-piperazineacetamide), LY 294002 hydrochloride (2-(4-Morpholinyl)-8-phenyl-4H-1-benzopyran-4-one hydrochloride), 3-Methyladenine (3-Methyl-3H-purin-6-amine), PF 04691502 (2-Amino-8-[trans-4-(2-hydroxyethoxy)cyclohexyl]-6-(6-methoxy-3-pyridinyl)-4-methyl-pyrido[2,3-d]pyrimidin-7 (8H)-one), PF 05212384 (N-[4-[[4-(Dimethylamino)-1-piperidinyl]carbonyl]phenyl]-N′-[4-(4,6-di-4-morpholinyl-1,3,5-triazin-2-yl)phenyl]urea), PI 103 hydrochloride (3-[4-(4-Morpholinylpyrido[3′,2′: 4,5]furo[3,2-d]pyrimidin-2-yl]phenol hydrochloride), PI 828 (2-(4-Morpholinyl)-8-(4-aminophenyl)-4H-1-benzopyran-4-one), PP 121 (1-Cyclopentyl-3-(1H-pyrrolo[2,3-b]pyridin-5-yl)-1H-pyrazolo[3,4-d]pyrimidin-4-amine), Quercetin, TG 100713 (3-(2,4-Diamino-6-pteridinyl)-phenol), Wortmannin, PIK90, and GDC-0941.
35. The method of form 34, wherein the PI3K pathway inhibitor is PIK90.
36. The method of any one of forms 2 to 35, wherein the TGF-beta pathway inhibitor is selected from the group consisting of A-83-01 (3-(6-Methyl-2-pyridinyl)-N-phenyl-4-(4-quinolinyl)-1H-pyrazole-1-carbothioamide), D4476 (4-[4-(2,3-Dihydro-1,4-benzodioxin-6-yl)-5-(2-pyridinyl)-1H-imidazol-2-yl]benzamide), GW 788388 (4-[4-[3-(2-Pyridinyl)-1H-pyrazol-4-yl]-2-pyridinyl]-N-(tetrahydro-2H-pyran-4-yl)-benzamide), LY 364947 (4-[3-(2-Pyridinyl)-1H-pyrazol-4-yl]-quinoline), RepSox (2-(3-(6-Methylpyridine-2-yl)-1H-pyrazol-4-yl)-1,5-naphthyridine), SB431542 (4-[4-(1,3-benzodioxol-5-yl)-5-(2-pyridinyl)-1H-imidazol-2-yl]benzamide), SB-505124 (2-[4-(1,3-Benzodioxol-5-yl)-2-(1,1-dimethylethyl)-1H-imidazol-5-yl]-6-methyl-pyridine), SB 525334 (6-[2-(1,1-Dimethylethyl)-5-(6-methyl-2-pyridinyl)-1H-imidazol-4-yl]quinoxaline), SD208 (2-(5-Chloro-2-fluorophenyl)-4-[(4-pyridyl)amino]pteridine), ITD1 (4-[1,1′-Biphenyl]-4-yl-1,4,5,6,7,8-hexahydro-2,7,7-trimethyl-5-oxo-3-quinolinecarboxylic acid ethyl ester), DAN/Fc, antibodies to TGF-beta and TGF-beta receptors, TGF-beta inhibitory nucleic acids.
37. The method of form 36, wherein the TGF-beta pathway inhibitor is A-83-01.
38. The method of any one of forms 2 to 37, wherein the BMP pathway inhibitor is selected from the group consisting of Chordin, soluble BMPR1a, soluble BMPR1b, Noggin, LDN-193189, and Dorsomorphin.
39. The method of form 38, wherein the BMP pathway inhibitor is LDN-193189.
40. The method of any one of forms 2 to 39, wherein the Wnt pathway inhibitor is selected from the group consisting of C59 (4-(2-Methyl-4-pyridinyl)-N-[4-(3-pyridinyl)phenyl]benzeneacetamide), DKK1, IWP-2 (N-(6-Methyl-2-benzothiazolyl)-2-[(3,4,6,7-tetrahydro-4-oxo-3-phenylthieno[3,2-d]pyrimidin-2-yl)thio]-acetamide), Ant1.4Br, Ant 1.4CI, Niclosamide, apicularen, bafilomycin, XAV939 (3,5,7,8-Tetrahydro-2-[4-(trifluoromethyl)phenyl]-4H-thiopyrano[4,3-d]pyrimidin-4-one), IWR-1 (4-(1,3,3a,4,7,7a-Hexahydro-1,3-dioxo-4,7-methano-2H-isoindol-2-yl)-N-8-quinolinyl-Benzamide), NSC668036 (N-[(1,1-Dimethylethoxy) carbonyl]-L-alanyl-(2S)-2-hydroxy-3-methylbutanoyl-L-Alanine-(1S)-1-carboxy-2-methylpropyl ester hydrate), 2,4-diamino-quinazoline, Quercetin, ICG-001 ((6S,9aS)-Hexahydro-6-[(4-hydroxyphenyl)methyl]-8-(1-naphthalenylmethyl)-4,7-dioxo-N-(phenylmethyl)-2H-pyrazino[1,2-a]pyrimidine-1 (6H)-carboxamide), PKF115-584, BML-284 (2-Amino-4-[3,4-(methylenedioxy)benzylamino]-6-(3-methoxyphenyl)pyrimidine), FH-535, iCRT-14, JW-55, JW-67, antibodies to Wnts and Wnt receptors, and Wnt inhibitory nucleic acids.
41. The method of form 40, wherein the Wnt pathway inhibitor is C59.
42. The method of any one of forms 2 to 41, wherein the MEK/ERK pathway inhibitor is selected from the group consisting of AP 24534 (3-(2-Imidazo[1,2-b]pyridazin-3-ylethynyl)-4-methyl-N-[4-[(4-methyl-1-piperazinyl)methyl]-3-(trifluoromethyl)phenyl]-benzamide), PD173074 (N-[2-[[4-(Diethylamino)butyl]amino]-6-(3,5-dimethoxyphenyl)pyrido[2,3-d]pyrimidin-7-yl]-N′-(1,1-dimethylethyl) urea), FIIN 1 hydrochloride (N-(3-((3-(2,6-dichloro-3,5-dimethoxyphenyl)-7-(4-(diethylamino)butylamino)-2-oxo-3,4-dihydropyrimido[4,5-d]pyrimidin-1 (2H)-yl)methyl)phenyl) acrylamide), PD 161570 (N-[6-(2,6-Dichlorophenyl)-2-[[4-(diethylamino)butyl]amino]pyrido[2,3-d]pyrimidin-7-yl]-N′-(1,1-dimethylethyl) urea), SU 5402 (2-[(1,2-Dihydro-2-oxo-3H-indol-3-ylidene)methyl]-4-methyl-1H-pyrrole-3-propanoic acid), SU 6668 (5-[1,2-Dihydro-2-oxo-3H-indol-3-ylidene)methyl]-2,4-dimethyl-1H-pyrrole-3-propanoic acid), PD0325901 (N-[(2R)-2,3-Dihydroxypropoxy]-3,4-difluoro-2-[(2-fluoro-4-iodophenyl)amino]-benzamide), BIX 02189 ((3Z)-3-[[[3-[(Dimethylamino)methyl]phenyl]amino]phenylmethylene]-2,3-dihydro-N,N-dimethyl-2-oxo-1H-indole-6-carboxamide), FR 180204 (5-(2-Phenyl-pyrazolo[1,5-a]pyridin-3-yl)-1H-pyrazolo[3,4-c]pyridazin-3-ylamine), Pluripotin (N-[3-[7-[(1,3-Dimethyl-1H-pyrazol-5-yl)amino]-1,4-dihydro-1-methyl-2-oxopyrimido[4,5-d]pyrimidin-3 (2H)-yl]-4-methylphenyl]-3-(trifluoromethyl)benzamide), TCS ERK 11e (4-[2-[(2-Chloro-4-fluorophenyl)amino]-5-methyl-4-pyrimidinyl]-N-[(1S)-1-(3-chlorophenyl)-2-hydroxyethyl]-1H-pyrrole-2-carboxamide), TMCB (2-(4,5,6,7-Tetrabromo-2-(dimethylamino)-1H-benzo[d]imidazol-1-yl) acetic acid), XMD 8-92 (2-[[2-Ethoxy-4-(4-hydroxy-1-piperidinyl)phenyl]amino]-5, 11-dihydro-5,11-dimethyl-6H-pyrimido[4,5-b][1,4]benzodiazepin-6-one), SU5402, AZD4547, BGJ398, AL 8697, AMG 548, CMPD-1, DBM 1285 dihydrochloride, EO 1428, JX 401, ML 3403, RWJ 67657, SB 202190, SB-203580, SB 239063, SB 706504, Scio-469, SKF 86002 dihydrochloride, SX 011, TA 01 (4-(2-(2,6-Difluorophenyl)-4-(fluorophenyl)-1H-imidazol-5-yl)pyridine), TA 02 (4-(2-(2-Fluorophenyl)-4-(fluorophenyl)-1H-imidazol-5-yl)pyridine), TAK 715, VX-702, and VX-745.
43. The method of form 42, wherein the MEK/ERK pathway inhibitor is PD0325901.
44. The method of any one of forms 2 to 43, wherein the Hedgehog pathway activator is selected from the group consisting of Hedgehog family ligands (Hh, Shh, Ihb, Dhb, etc.) and fragments thereof, benzothiophene smoothened agonists, SAG (Hh-Ag1.3), SAG21k (3-chloro-4,7-difluoro-N-(4-methoxy-3-(pyridin-4-yl)benzyl)-N-((1r,4r)-4-(methylamino)cyclohexyl)benzo[b]thiophene-2-carboxamide), Hh-Ag1.1, Hh-Ag1.5, and purmorphamine.
45. The method of form 44, wherein the Hedgehog pathway activator is purmorphamine.
46. A method of producing a population of osteoblasts or a bone-like organoid, the method comprising:
   a) deriving hypertrophic chondrocytes, hypertrophic chondrocyte-like cells, cartilage-like tissue, cartilage, according to the method of any one of forms 1 to 13, 17 to 20, 22 to 26 or 28 to 45; and
   b) culturing in orbital culture the hypertrophic chondrocytes, hypertrophic chondrocyte-like cells, hypertrophic cartilage-like tissue, hypertrophic cartilage with an osteogenic culture medium to generate osteoblasts or a bone-like organoid that express COL1A1 and COL1A2.
47. The method of form 46 wherein said osteogenic differentiation culture medium comprises β-glycerophosphate, ascorbic acid 2-phosphate, sodium ascorbate, and dexamethasone.
48. The method of form 46 or 47, wherein said osteogenic differentiation culture medium comprises a WNT agonist for about the first 3-7 days of culture.
49. The method of form 48, wherein said WNT agonist is CHIR99021.
50. The method of any one of forms 1-45, further comprising a step of decellularizing said three-dimensional aggregation of chondrocytes, or chondrocyte-like cells, cartilage-like tissue or cartilage to produce a decellularized scaffold.
51. The method of any one of forms 46-49, further comprising a step of decellularizing said bone-like organoid produce a decellularized scaffold.
52. The method of any one of forms 46-49, further comprising a step of degrading the extracellular matrix and isolating said osteoblasts.
53. Chondrocytes, Chondrocyte-like cells, cartilage-like tissue, cartilage, or a combination thereof produced according to the methods of any one of forms 1 to 45.
54. Articular (non-hypertrophic) chondrocytes or articular chondrocyte-like cells, articular cartilage-like tissue, articular cartilage or a combination thereof produced according to the methods of any one of forms 15 to 45.
55. Osteoblasts or bone-like organoid produced according to the method of any one of forms 46 to 49 or 52.
56. A decellularized scaffold prepared according to the method of form 50 or 51.
57. The method of any one of forms 1-45, further comprising administering the chondrocytes, chondrocyte-like cells, cartilage-like tissue, or cartilage, or a combination thereof to a subject.
58. The method of any one of forms 46-49 or 52, further comprising administering the osteoblasts, or bone-like organoid, or a combination thereof to a subject.
59. The method according to form 57 or 58 wherein said sclerotome cells are derived from said subject.
60. A composition comprising a homogenous population of iPSC-derived chondrocytes, wherein said cells express collagen 2 (COL2A1) and ACAN at a ratio in the range of 20:1 to 5:1, preferably about 10:1 and does not substantially express COL10A1.
61. A composition comprising a homogenous population of iPSC-derived hypertrophic chondrocytes, wherein said cells express collagen 2 (COL2A1) and COL10A1 at a ratio in the range of 1:1 to about 2.5:1.
62. A method of treating a chondral defect or an osteochondral defect in a subject in need thereof comprising a) generating a composition comprising: chondrocytes, chondrocyte-like cells, cartilage-like tissue, cartilage, or a combination thereof produced according to any one of forms 1 to 44, articular (non-hypertrophic) chondrocytes or articular chondrocyte-like cells, articular cartilage-like tissue, articular cartilage according to any one of forms 15 to 45 and/or osteoblasts or bone-like organoid according to forms 46 to 49 or 52, and b) administering the composition to the subject.
63. A method of testing candidate chondrogenic or osteogenic modulating substances, the method comprising:
   a) carrying out the method of any one of forms 1-49, wherein said test substance is included in any one, or more, of the culture steps of the method;
   b) assessing the effect of the test substance on chondrocyte or osteoblast proliferation, maintenance and/or differentiation compared to a control population generated in the absence of test substance; and
   c) identifying the test substance as a candidate chondrogenic or osteogenic modulating substance if the test substance increases or decreases proliferation, and/or affects chondrocyte or osteoblast maintenance or differentiation compared to the control.
64. The method of form 63, wherein the method of any one of forms 1-52 is carried out using cells derived from a subject with a bone or cartilage disease or disorder, or cells engineered to have a mutation associated with a bone or cartilage disease or disorder or engineered to correct a mutation associated with a bone or cartilage disease or disorder.
65. The method of form 64, wherein the bone disorder is osteogenesis imperfecta.
66. The method of form 64, wherein the cartilage disorder is associated with a mutation in COL2A1.
67. The method of any one of forms 1-52 wherein the cells are derived from a subject with a bone or cartilage disease or disorder, or cells engineered to have a mutation associated with a bone or cartilage disease or disorder, or engineered to correct a mutation associated with a bone or cartilage disease or disorder.
68. Use of chondrocytes, chondrocyte-like cells, cartilage-like tissue, cartilage, or a combination thereof produced according to any one of forms 1 to 45, articular (non-hypertrophic) chondrocytes or articular chondrocyte-like cells, articular cartilage-like tissue, articular cartilage according to any one of forms 15 to 45 and/or osteoblasts or bone-like organoid according to forms 46 to 49 or 52, for disease modelling or testing therapeutic agents for a bone or cartilage disease or disorder.

### BRIEF DESCRIPTION OF DRAWINGS

FIG. 1 shows directed iPSC differentiation to skeletal cells. (A) Schematic representation of the differentiation stages from iPSCs to articular and hypertrophic chondrocytes then towards osteoblast-like cells. The process recapitulates embryonic development with differentiation to sclerotome in the first 6 days including aggregation of the monolayer cells into pellets at day 4. Culture medium and conditions are shown below and above the timeline and the culture formats are illustrated at the bottom. (B) Gene expression of stage specific markers during the 6-day differentiation to sclerotome, OCT4, MIXL1, MSGN1, MEOX1 and PAX1, and the chondrocyte markers, SOX9 and COL2A1, in iPSC line MCRIi019-A. N=4 technical replicates.

FIG. 1.1 shows gene expression of stage specific markers during the 6-day differentiation to sclerotome, OCT4, MIXL1, MSGN1, MEOX1 and PAX1, and the chondrocyte markers, SOX9 and COL2A1, in iPSC line MCRIi018-B. N=3 technical replicates.

FIG. 2 shows optimizing sclerotome to chondrocyte differentiation. (A) The iPSC line MCRIi018-B was differentiated to sclerotome with cell pellets formed at day 4. At the end of day 6 sclerotome pellets were supplemented with either 20 ng/ml BMP4 or 20 ng/ml FGF2 for 14 days. Toluidine blue stained pellet sections at day 20, 34, and 48. FGF2 supplemented pellets differentiated to chondrocytes by day 34 while BMP4 treated pellets remained small with isolated toluidine positive areas. Scale bar is 500 μm. (B) Relative mRNA expression of cartilage genes COL2A1, ACAN, and COL10A1. N=2 or 3 technical replicates. (C) Collagen II and X immunostaining. FGF2 treated pellets had deposited an extensive collagen II extracellular matrix by day 48. BMP4 treated pellets contained only small patches of collagen II and collagen X expressing cells. Scale bar is 200 μm. (D) Rotary culture influences chondrocyte maturity. The iPSC line MCRIi001-A-2 was differentiated to sclerotome with cell pellets formed at day 4. At the end of day 6 sclerotome pellets were supplemented with 20 ng/ml FGF2 for 14 days. Some pellets were transferred to rotary culture at the end of day 6, day 13, day 20 or day 27 and some remained in static culture. Toluidine blue stained pellets at day 34 and 48. Collagen X immunostaining at day 48 showing that pellets grown in rotary culture from day 13 and day 20 have matured to collagen X expressing hypertrophic chondrocytes, while static pellets and pellets transferred to rotary culture on day 6 do not express collagen X. Scale bars are 500 μm.

FIG. 2.1 shows enhanced chondrocyte differentiation in feeder-free iPSC lines. Four iPSC lines, MCRIi018-B (feeder-free), RM3.5 (feeder-dependent), MCRIi001-A and MCRIi001-A-2 (isogenic feeder-dependent lines), were differentiated to sclerotome with cell pellets formed at day 4. At the end of day 6 sclerotome pellets were supplemented with 20 ng/ml FGF2 for 14 days then harvested for RNAseq. N=3 technical replicates. (A) Principal component analysis. (B) Volcano plots illustrate the large number of differentially expressed genes in the feeder-free v feeder-dependent comparisons and indicate that signature cartilage genes COL2A1, COL9A1, COL11A1, ACAN and SOX9, are more highly expressed in the feeder-free line. There are fewer differentially expressed genes in the feeder-dependent comparisons and differentially expressed genes between the isogenic lines are rare. (C) MCRIi001-A-2 was adapted to feeder free conditions, then feeder-dependent and feeder-free versions differentiated to chondrocytes as before with pellets transferred to rotary culture at the end of day 6. Toluidine blue stained pellets at day 34, 48 and 62. At the end of the differentiation, pellets from feeder-dependent MCRIi001-A-2 contained a mixture of cartilage and non-cartilage tissue while pellets from feeder-free MCRIi001-A-2 were predominantly cartilage with just a thin layer of non-cartilage tissue on the outside of the pellet. Scale bar is 500 μm.

FIG. 2.2 shows rotary culture influences chondrocyte maturity. The iPSC line MCRIi019-A was differentiated to sclerotome with cell pellets formed at day 4. At the end of day 6 sclerotome pellets were supplemented with 20 ng/ml FGF2 for 14 days. Some pellets were transferred to rotary culture at the end of day 6, day 13, day 20 or day 27 and some remained in static culture. Toluidine blue stained MCRIi019-A pellets at day 34 and 48. At day 48 pellets grown in rotary culture from day 6 are surrounded by a layer of non-cartilage tissue and pellets grown in static culture have reduced toluidine blue staining suggesting proteoglycan breakdown. Scale bar is 500 μm. Collagen X immunostaining at D48 shows that pellets transferred to rotary culture at D13 have a collagen X rich ECM. Scale bar is 200 μm.

FIG. 3 shows maturing to growth plate cartilage. Chondronoids maintained in chondrogenic medium spontaneously mature towards hypertrophy and hypertrophy can be enhanced with T3. (A) Toluidine blue stained chondronoid sections (MCRIi019-A) at day 34, 48 and 69. Some pellets were treated with T3 for 3 weeks before harvesting at day 69. The cells become visibly larger between day 48 and day 69 suggesting they are maturing towards hypertrophic chondrocytes. Scale bar is 500 μm. (B) Collagen II and X immunostaining. The chondronoids contain an extensive collagen II rich ECM throughout the time course. Collagen X is not apparent at day 48 but has been deposited into the ECM by day 69. Scale bar is 200 μm. (C) Electron microscopy of a chondronoid at day 52 showing an extensive network of collagen II fibrils in the ECM. (D) Heatmap showing changes in mRNA expression of selected cartilage, hypertrophic cartilage and bone proteins. Heirarchical clustering shows that hypertrophic cartilage and bone markers are more highly expressed with time and expression is further enhanced by T3, while canonical cartilage genes are downregulated with time and T3 treatment. N=3 independent differentiation experiments.

FIG. 3.1 shows maturing to growth plate cartilage. Chondronoids maintained in chondrogenic medium spontaneously mature towards hypertrophy and hypertrophy can be enhanced with T3. (A) Toluidine blue stained chondronoid sections (MCRIi018-B) at day 34, 48 and 69. Some pellets were treated with T3 for 3 weeks before harvesting at day 69. The cells become visibly larger between day 48 and day 69 suggesting they are maturing towards hypertrophic chondrocytes. Scale bar is 500 μm. (B) Collagen II and X immunostaining. The chondronoids contain an extensive collagen II rich ECM throughout the time course. Collagen X is not apparent at day 48 but has been deposited into the ECM by day 69. Scale bar is 200 μm. (C) Heatmap showing changes in mRNA expression of selected cartilage, hypertrophic cartilage and bone proteins in iPSC line MCRIi018-B during chondrocyte maturation to hypertrophy. Heirarchical clustering shows that hypertrophic cartilage and bone markers are more highly expressed with time and expression is further enhanced by T3, while canonical cartilage genes are downregulated with time and T3 treatment. N=3 independent differentiation experiments.

FIG. 3.2 shows most highly expressed core matrisome genes. Lists of the 20 core matrisome genes with the highest RPKM values at day 48, day 69 and day 69+T3 were combined and gene expression (log 2 RPKM) in all samples was plotted. (A) MCRIi019-A. (B) MCRIi018-B. Twenty five of the 27 most highly expressed genes in each cell line were identical and the gene expression patterns between day 48, day 69 and day 69+T3 were similar.

FIG. 4 shows hypertrophy related gene set enrichment. Gene Set Enrichment Analysis (GSEA) of the RNAseq D69T3 v D48 comparison identified MSigDB gene sets relevant to cartilage maturation. (A) Hallmark gene sets indicate reduced cell division in hypertrophic chondrocytes and enrichment in hypoxia and apoptosis pathways. (B) Enriched sets from the MSigDB C2 (curated) gene sets included NABA_CORE_MATRISOME and other matrisome gene sets. These matrisome gene sets have members that are highly upregulated and members that are highly downregulated reflecting dynamic changes in the ECM during chondrocyte hypertrophy. (C) Heatmap showing relative expression of the NABA_SECRETED_FACTORS that were differentially expressed in the D69T3 v D48 comparison (Adj.P.value <0.05, Log FC ≥1 or ≤−1) and expressed at average RPKM >4 at one or more cell line/day/treatment. Genes in the GOBP_BLOOD_VESSEL_MORPHOGENESIS, KEGG_TGF_BETA_SIGNALING_PATHWAY and KEGG_WNT_SIGNALING_PATHWAY gene sets are indicated on the right.

FIG. 5. Transcription factors dynamically regulated during maturation to hypertrophy. Transcription factors differentially expressed in the RNAseq D69T3 v D48 comparison (adj. P. value≤0.05, Log FC ≥1 or ≤−1) and expressed at average RPKM >10 at one or more cell line/day/treatment are shown grouped by the direction of change and their known or unknown role in cartilage development. The points show the average log 2 RPKM for each group, N=3 biological replicates.

FIG. 5.1 shows interactions between transcription factors differentially expressed during maturation to hypertrophy. (A) STRING analysis (https://string-db.org) identified multiple protein-protein interactions and interaction nodes between transcription factors upregulated during hypertrophy (adj. P. value≤0.05, Log FC ≥1 or ≤−1, and expressed at average RPKM >4 at one or more cell line/day/treatment). (B) STRING analysis of transcription factors downregulated in hypertrophy.

FIG. 6 shows TGFβ3 induces an articular chondrocyte phenotype. The iPSC line MCRIi019-A was differentiated to sclerotome with cell pellets formed at day 4. At the end of day 6 all sclerotome pellets were supplemented with 20 ng/ml FGF2 for 14 days and some pellets were treated with 10 ng/ml TGFβ3 from day 13. Pellets were transferred to rotary culture at day 20. (A) Histology and immunostaining at day 48. Untreated pellets contained large hypertrophic chondrocytes that had deposited a collagen II and collagen X containing extracellular matrix but did not express the articular cartilage protein PRG4. The chondrocytes in TGFβ3 treated pellet were small and the extracellular matrix contained collagen II and PRG4 but not collagen X. Scale bars are 500 μm. (B) Graph showing mRNA expression (log 2 RPKM) of the 20 core matrisome components that are the most highly expressed at day 48 in TGFβ3 treated chondronoids. Expression in untreated chondronoids is shown for comparison. (C) Expression at day 48 in TGFβ3 treated chondronoids (average log 2 RPKM) of the 20 most upregulated and 20 most downregulated core matrisome genes (log FC, adj.P.value <0.05, average RPKM >10 in at least one treatment group). (D) Graph showing mRNA expression (log 2 RPKM) of the 20 transcription factors that are the most highly expressed at day 48 in TGFβ3 treated chondronoids. Expression in untreated chondronoids is shown for comparison. (E) Expression at day 48 in TGFβ3 treated chondronoids (average log 2 RPKM) of the 20 most upregulated and 20 most downregulated transcription factor genes (log FC, adj.P.value <0.05, average RPKM >10 in at least one treatment group). RNAseq data N=4 technical replicates.

FIG. 7 shows hypertrophic chondrocyte to osteoblast transdifferentiation. (A) Transdifferentiation in vivo. The iPSC line MCRIi001-A-BFP was differentiated to sclerotome with pellets formed at day 4. From the end of day 6 cultures were supplemented with 20 ng/ml FGF2 for 14 days. From day 35 cultures were treated with 10 nM T3 for 7 days then, at day 42, hypertrophic chondronoids were implanted subcutaneously into immunocompromised mice, then harvested after 13 weeks. Implants were decalcified then sectioned and stained with safranin O for cartilage proteoglycans and fast green to highlight bone. Scale bar on left hand image is 1000 μm. The box shows the region enlarged in the middle image. The image on the right shows the same region from an adjacent section immunostained with a human specific Ku80 antibody. Scale bars on middle and right images are 100 μm. (B) Transdifferentiation in vitro. MCRIi018-B was differentiated to sclerotome with pellets formed on day 4. At the end of day 6 pellets were supplemented with 20 ng/ml FGF2 for two weeks then transferred to rotary culture. From day 38 cultures were treated with 10 nM T3 for 14 days (D52+T3) then transferred to either osteogenic medium for a further 3 weeks (D73 osteo), or osteogenic medium supplemented with 3 μM CHIR99021 for 7 days followed by culture in osteogenic medium alone (D73 osteo+CHIR). Toluidine blue staining is reduced in osteo+CHIR treated organoids, collagen I is deposited into the ECM in both osteogenic conditions and positive von Kossa staining indicates that calcium is deposited into the ECM in both osteogenic conditions. Scale bars are 500 μm. (C) Organoids were fixed then microCT scanned. Representative images are shown and these are the samples shown in red in the two graphs. Scale bar is 200 μm. Graphs show that while the organoids grown in the two conditions are similar in size, significant calcium phosphate mineral is only apparent in organoids grown in osteogenic conditions without CHIR99021 supplementation.

FIG. 7.1 shows T3 treatment primes for transdifferentiation to osteoblasts. MCRIi001-A-2 were differentiated to sclerotome with pellets formed on day 4. From the end of day 6 pellets were supplemented with 20 ng/ml FGF2 for 14 days then transferred to rotary culture and allowed to mature until day 68. Some pellets were then treated with 10 nM T3 for 14 days. From day 82 transdifferentiation was induced with either osteogenic medium alone for 3 weeks (osteo), or with osteogenic medium supplemented with 3 μM CHIR99021 for 7 days followed by culture in osteogenic medium alone (osteo+CHIR). Toluidine blue staining shows loss of proteoglycans was greater in CHIR99021 treated organoids. In both osteogenic differentiation conditions there was more extensive and intense von Kossa staining in organoids that had been pre-treated with T3 compared to those without T3 treatment. In T3 pre-treated pellets there was more intense von Kossa staining in pellets transdifferentiated in osteogenic medium alone than those transdifferentiated in osteogenic medium with CHIR99021 supplementation for the first 7 days. Scale bar is 500 μm.

FIG. 7.2 shows further evidence of transdifferentiation in vitro. From day 38 MCRIi018-B chondronoids were treated with 10 nM T3 for 14 days (T3) then transferred to osteogenic medium for a further 3 weeks. A, Relative expression (RPKM) of cartilage genes COL2A1 and ACAN declines, and osteoblast/osteocyte genes COL1A1, BGLAP, SPP1 and DMP1 significantly increases in osteogenic conditions. B, Immunostaining shows collagen I and BGLAP deposited in the ECM in osteogenic conditions (scale bar is 200 μm). Calcium phosphate mineral is deposited in oseogenic medium (uCT, scale bar is 500 μm).

FIG. 8 shows gene expression changes during hypertrophic chondrocyte to osteoblast transdifferentiation. MCRIi018-B was differentiated to hypertrophic chondrocytes (T3) then transferred to either osteogenic medium for a further 3 weeks (D73 O), or osteogenic medium supplemented with 3 μM CHIR99021 for 7 days followed by culture in osteogenic medium alone (D73 OC). (A) Graphs showing mRNA expression (RPKM) of key hypertrophic cartilage markers, COL2A1, ACAN and COL10A1, and genes highly expressed in pre-osteoblasts and osteoblasts, COL1A1, SPP1 and IBSP. (B) Graph showing mRNA expression (log 2 RPKM) of the 20 core matrisome components that are the most highly expressed in D73 OC treated organoids. Expression in D73 O, and D52 T3 treated samples are shown for comparison. (C) Expression in D73 OC treated organoids (average log 2 RPKM) of the 20 most upregulated and 20 most downregulated core matrisome genes (log FC, adj.P.value <0.05, average RPKM >10 in at least one treatment group). (D) Graph showing mRNA expression (log 2 RPKM) of the 20 transcription factors that are the most highly expressed in D73 OC treated organoids. Expression in D73 O, and D52 T3 treated samples are shown for comparison. (E) Expression in D73 OC treated organoids (average log 2 RPKM) of the 20 most upregulated and 20 most downregulated transcription factor genes (log FC, adj.P.value <0.05, average RPKM >10 in at least one treatment group). RNAseq data n=4 technical replicates.

FIG. 8.1 shows gene expression changes during hypertrophic chondrocyte to osteoblast transdifferentiation. MCRIi019-A was differentiated to hypertrophic chondrocytes (T3) then transferred to either osteogenic medium for a further 3 weeks (D72 O), or osteogenic medium supplemented with 3 μM CHIR99021 for 7 days followed by culture in osteogenic medium alone (D72 OC). (A) Graphs showing mRNA expression (RPKM) of key hypertrophic cartilage markers, COL2A1, ACAN and COL10A1, and genes highly expressed in pre-osteoblasts and osteoblasts, COL1A1, SPP1, IBSP and BGLAP. (B) Graph showing mRNA expression (log 2 RPKM) of the 20 core matrisome components that are the most highly expressed in D72 OC treated organoids. Expression in D72 O, and D51 T3 treated samples are shown for comparison. (C) Expression in D72 OC treated organoids (average log 2 RPKM) of the 20 most upregulated and 20 most downregulated core matrisome genes (log FC, adj.P.value <0.05, average RPKM >10 in at least one treatment group). (D) Graph showing mRNA expression (log 2 RPKM) of the 20 transcription factors that are the most highly expressed in D72 OC treated organoids. Expression in D72 O, and D51 T3 treated samples are shown for comparison. (E) Expression in D72 OC treated organoids (average log 2 RPKM) of the 20 most upregulated and 20 most downregulated transcription factor genes (log FC, adj.P.value <0.05, average RPKM >10 in at least one treatment group). RNAseq data n=4 technical replicates.

FIG. 9 shows expression of genes that mark osteoblast lineage cells in vivo. MCRIi018-B was differentiated to hypertrophic chondrocytes (T3) then transferred to either osteogenic medium for a further 3 weeks (O), or osteogenic medium supplemented with 3 μM CHIR99021 for 7 days followed by culture in osteogenic medium alone (OC). Graph shows expression of genes (log 2 RPKM) that defined osteoblast precursor, osteoblast and more mature osteoblast cell clusters in scRNAseq of cells isolated from mouse bone (Ayturk et al., 2020). The log fold change in expression (log FC) relative to hypertrophic chondrocytes is shown on the right. The genes that mark mature osteoblasts are highly expressed in osteogenic culture conditions (O and OC) and many are highly upregulated compared to hypertrophic chondrocytes (T3). RNAseq data N=4 technical replicates.

FIG. 9.1 shows gene expression during hypertrophic chondrocyte to osteoblast transdifferentiation. MCRIi018-B was differentiated to hypertrophic chondrocytes (T3) then transferred to either osteogenic medium for 3 weeks (D73 O), or osteogenic medium supplemented with 3 μM CHIR99021 for 7 days followed by 2 weeks culture in osteogenic medium alone (D73 OC). (A) Expression of key in vivo markers of skeletal lineage cells. The gene list is taken from single-cell RNAseq data derived from mouse pup distal femur and proximal tibia epiphyses and includes the genes that marked hypertrophic chondrocyte, pre-osteoblast and osteoblast cell clusters (Haseeb et al., 2021). In osteogenic culture conditions (O and OC) all but two genes, MGP and SOX4, follow the expression pattern identified during in vivo transdifferentiation. Nine out of 11 genes are more highly upregulated in OC treated organoids than in O treated organoids (compared to T3 treated, hypertrophic chondrocytes). All five chondrocyte marker genes are more downregulated in OC treated than O treated organoids. (B) Expression of key osteoblast transcription factors. The gene list is taken from a recent review (Chan et al., 2021). All these osteogenic transcription factors are expressed in osteogenic culture conditions (O and OC), and 12/13 are upregulated compared to hypertrophic chondrocytes (T3). All these osteogenic transcription factors are expressed at a similar level or higher in organoids exposed to a CHIR pulse (OC) than when differentiated in osteogenic medium alone (O). RNAseq data N=4 technical replicates.

FIG. 10 shows disease modeling using chondrocyte and osteoblast organoids differentiated from human iPSC. Panels A and B: Using gene-edited iPSC lines with an inherited cartilage disease (hypochondrogenesis) COL2A1 p.G1113C mutation and isogenic control (Lilianty J, Bateman J F, Lamandé SR. Stem Cell Res. 2021 Aug. 25; 56:102515) iPSC were differentiated into mature chondrocytes using our protocol. The collagen II extracellular matrix was assessed by collagen II immunohistochemistry (A) and by electron microscopy (B). Both methods demonstrated the reduced collagen II extracellular matrix in the hypochondrogenesis mutant. Scale bars=200 μm (A) and 500 nm (B). In the hypochondrogenesis iPSC-derived cartilage organoid (A) the pathological intracellular accumulation of the mutant misfolded mutant collagen II is indicated by arrows. In panel B the poor formation of the cartilage collagen II extracellular matrix in the hypochondrogenesis iPSC-derived cartilage organoid is visualized by electron microscopy. Scale bar=500 nm. Panels C-F: Using gene-edited iPSC lines with an inherited osteogenesis imperfecta, COL1A1 p.W1312C mutation, and isogenic control (Howden S, et al., Stem Cell Res. 2019 July; 38:101453) iPSC were differentiated into osteoblasts using our protocol. The osteogenesis imperfecta iPSC-derived osteoblasts show reduced collagen I extracellular matrix formation by immunohistochemistry (C), and reduced calcification by von Kossa staining (D). Scale bar=500 μm. The reduced calcification (bone formation) of the osteogenesis imperfecta bone organoid is more dramatically shown by microCT analysis (E). Scale bar=100 μm. The quantification of the microCT data is shown in (F). N=5.

### DESCRIPTION OF EMBODIMENTS

### Definitions

Definitions of common terms in cellular and molecular biology, and biochemistry can be found in The Merck Manual of Diagnosis and Therapy, 20th Edition, published by Merck Sharp & Dohme Corp., 2018 (ISBN 9780911910421, 0911910425); Robert S. Porter et al. (eds.), The Encyclopedia of Molecular Cell Biology and Molecular Medicine, published by Blackwell Science Ltd., 2008 (ISBN 3527305424, 9783527305421); and Robert A. Meyers (ed.), Molecular Biology and Biotechnology: a Comprehensive Desk Reference, published by VCH Publishers, Inc., 1995 (ISBN 1-56081-569-8); Immunology by Wemer Luttmann, published by Elsevier, 2006; Janeway's Immunobiology, Kenneth Murphy, Allan Mowat, Casey Weaver (eds.), Taylor & Francis Limited, 2016 (ISBN 9780815345510, 0815345518); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al, Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Laboratory Methods in Enzymology: RNA, Jon Lorsch (ed.) Elsevier, 2013 (ISBN: 9780124200371, 0124200370); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, A D A M Kruisbeek, David H Margulies, Ethan M Shevach, Warren Strobe, (eds.) John Wiley and Sons, Inc., 2003 (ISBN 0471142735, 9780471142737), Immunological Methods, Ivan Lefkovits, Benvenuto Pemis, (eds.) Elsevier Science, 2014 (ISBN: 9781483269993, 148326999X), the contents of which are all incorporated by reference herein in their entireties.

As used in this specification and the appended claims, terms in the singular and the singular forms “a,” “an” and “the,” for example, optionally include plural referents unless the content clearly dictates otherwise. For example, “a” cell includes one cell, one or more cells and a plurality of cells.

As used herein, the term “about”, unless stated to the contrary, refers to +/−10%, more preferably +/−5%, more preferably +/−1%, of the designated value. Accordingly, when used in conjunction with a designated value, e.g. “about X” shall be understood to mean designated value itself or the designated value+/−10%, more preferably +/−5%, more preferably +/−1%, of the designated value.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Numeric ranges are inclusive of the numbers defining the range. It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The headings provided herein are not intended to limit the disclosure.

Throughout the specification, references to particular genes or proteins may be used interchangeably. A person skilled in the art will understand in the context whether the reference is intended to be a reference to the particular gene or the protein that is encoded by that gene.

The terms “human pluripotent stem cell” and “hPSC” refer to cells derived, obtainable or originating from human tissue that display pluripotency. The hPSC may be a human embryonic stem cell or a human induced pluripotent stem cell.

Human pluripotent stem cells may be derived from inner cell mass or reprogrammed using Yamanaka factors from many fetal or adult somatic cell types. The generation of hPSCs may be possible using somatic cell nuclear transfer.

The terms “human embryonic stem cell”, “hES cell” and “hESC” refer to cells derived, obtainable or originating from human embryos or blastocysts, which are self-renewing and pluri- or toti-potent, having the ability to yield all of the cell types present in a mature animal. Human embryonic stem cells (hESCs) can be isolated, for example, from human blastocysts obtained from human in vivo preimplantation embryos, in vitro fertilized embryos, or one-cell human embryos expanded to the blastocyst stage.

The terms “induced pluripotent stem cell” and “iPSC refer to cells derivable, obtainable or originating from human adult somatic cells of any type reprogrammed to a pluripotent state through the expression of exogenous genes, such as transcription factors, including a preferred combination of OCT4, SOX2, KLF4 and c-MYC. hiPSC show levels of pluripotency equivalent to hESC but can be derived from a patient for autologous therapy with or without concurrent gene correction prior to differentiation and cell delivery.

More generally, the method disclosed herein could be applied to any pluripotent stem cell derived from any patient or a hPSC subsequently modified to generate a mutant model using gene-editing or a mutant hPSC corrected using gene-editing. Gene-editing could be by way of CRISPR, TALEN or ZF nuclease technologies.

As used herein, the term “cell culture” refers to any in vitro culture of cells. The term “culturing” refers to the process of growing and/or maintaining and/or manipulating a cell. Included within this term are continuous cell lines (e.g., with an immortal phenotype), primary cell cultures, finite cell lines (e.g., non-transformed cells), and any other cell population maintained in vitro, including oocytes and embryos. As used herein, the terms “primary cell culture,” and “primary culture,” refer to cell cultures that have been directly obtained from cells in vivo, such as from a tissue specimen or biopsy from an animal or human. These cultures may be derived from adults as well as fetal tissue.

A “progenitor cell” is a cell which is capable of differentiating along one or a plurality of developmental pathways, with or without self-renewal. Typically, progenitor cells are unipotent or oligopotent and are capable of at least limited self-renewal.

The terms “differentiate”, “differentiating” and “differentiated”, relate to progression of a cell from an earlier or initial stage of a developmental pathway to a later or more mature stage of the developmental pathway. It will be appreciated that in this context “differentiated’ does not mean or imply that the cell is fully differentiated and has lost pluripotency or capacity to further progress along the developmental pathway or along other developmental pathways. Differentiation may be accompanied by cell division.

As will be well understood in the art, the stage or state of differentiation of a cell may be characterized by the expression and/or non-expression of one of a plurality of markers. In this context, by “markers” is meant nucleic acids or proteins that are encoded by the genome of a cell, cell population, lineage, compartment or subset, whose expression or pattern of expression changes throughout development. Nucleic acid marker expression may be detected or measured by any technique known in the art including nucleic acid sequence amplification (e.g. polymerase chain reaction) and nucleic acid hybridization (e.g. microarrays, Northern hybridization, in situ hybridization), although without limitation thereto. Protein marker expression may be detected or measured by any technique known in the art including flow cytometry, immunohistochemistry, immunoblotting, protein arrays, protein profiling (e.g. 2D gel electrophoresis), although without limitation thereto.

Such terms are commonplace and well-understood by the skilled person when characterizing cell phenotypes. By means of additional guidance, when a cell is said to be positive for or to express or comprise expression of a given marker, such as a given gene or gene product, a skilled person would conclude the presence or evidence of a distinct signal for the marker when carrying out a measurement capable of detecting or quantifying the marker in or on the cell. Suitably, the presence or evidence of the distinct signal for the marker would be concluded based on a comparison of the measurement result obtained for the cell to a result of the same measurement carried out for a negative control (for example, a cell known to not express the marker) and/or a positive control (for example, a cell known to express the marker). Where the measurement method allows for a quantitative assessment of the marker, a positive cell may generate a signal for the marker that is at least 1.5-fold higher than a signal generated for the marker by a reference cell (e.g. negative control cell) or than an average signal generated for the marker by a population of reference or negative control cells, e.g., at least 2-fold, at least 4-fold, at least 10-fold, at least 20-fold, at least 30-fold, at least 40-fold, at least 50-fold higher, at least 100-fold higher, or even higher. Further, a positive cell may generate a signal for the marker that is 3.0 or more standard deviations, e.g., 3.5 or more, 4.0 or more, 4.5 or more, or 5.0 or more standard deviations, higher than an average signal generated for the marker by a population of reference or negative control cells.

As used herein “sclerotome cells” or “sclerotome” may be used interchangeably and refers to an iPSC-derived population that is enriched for cells that are functionally and phenotypically similar to the part of the somite in a vertebrate embryo that gives rise to bone or other skeletal tissue. In some embodiments, “sclerotome cells” or “sclerotome” express any one of PAX1, SOX9, FOXC2, PAX9, NKX3.2/BAPX1 and TWIST1. In some embodiments the scleretome cells express PAX1, SOX9 and FOXC2, and may also express one or more further markers selected from PAX9, NKX3.2/BAPX1 and TWIST1.

As used herein “chondrocyte progenitor cells” and “chondrocyte progenitors” and “chondroprogenitors” are used interchangeably and refer to progenitor cells derived from sclerotome cells that are specifically pre-disposed to differentiate into chondrocytes. In some embodiments, “chondrocyte progenitor cells” and “chondrocyte progenitors” and “chondroprogenitors” may express some or all of the genes expressed by chondrocytes. In some embodiments, “chondrocyte progenitor cells”, “chondrocyte progenitors” and “chondroprogenitors” express about 10- to 50-fold less COL2A1 and about 50- to 200-fold less ACAN (relative to a housekeeping gene) when compared to a chondrocyte.

As used herein “chondrocyte”, “chondrocytes”, and “chondrocyte cells” refer to cells derived from chondrocyte progenitors. In some embodiments, “chondrocyte”, “chondrocytes”, and “chondrocyte cells” express COL2A1 and ACAN at a ratio in the range of about 10:1 (or from 20:1 to 5:1) COL2A1: ACAN, and do not substantially express COL10A1 . . . . In some embodiments, “chondrocyte”, “chondrocytes”, and “chondrocyte cells” express COL2A1 and ACAN and one or more further genes selected from the group consisting of COL11A1, COL11A2, COL9A1, COL9A2, COL9A3, MATN1 and MATN3. “Chondrocyte-like cells” refer to cells that substantially reflect the chondrocyte phenotype or are functionally equivalent to chondrocytes.

As used herein “hypertrophic chondrocytes” and “hypertrophic cartilage” refer to cells that expresses COL2A1 and COL10A1. In some embodiments the hypertrophic chondrocytes and hypertrophic cartilage-like tissue, hypertrophic cartilage, or a combination thereof expresses COL2A1 and COL10A1 at a ratio in the range of about 1:1 to about 2.5:1. “Hypertrophic chondrocyte-like cells” and “hypertrophic cartilage-like tissue”, refers to cells and tissues that substantially reflect the hypertrophic chondrocyte phenotype or are functionally equivalent to hypertrophic chondrocytes.

As used herein “articular chondrocytes” and “articular cartilage” refers to cells that express PRG4. In some embodiments “articular chondrocytes” and “articular cartilage” express COL2A1, ACAN and PRG4, and optionally, one or more further genes selected from the group consisting of ASPN, CILP, CILP2. In some embodiments “articular chondrocytes” and “articular cartilage” also express one of more of EMILIN1, EMILIN3, FBLN1, and FBLN3. “Articular chondrocyte-like cells”, “articular cartilage-like tissue”, refers to cells and tissues that substantially reflect the articular chondrocyte phenotype or are functionally equivalent to articular chondrocytes or articular cartilage respectively.

As used herein “osteoblasts” or a bone-like organoid express COL1A1 and COL1A2. In some embodiments “osteoblasts” or a bone-like organoid also express one or more of MEPE, IBSP (bone sialoprotein 2), SPP1 (osteopontin) and DMP1.

As used herein, the terms “culture medium,” and “cell culture medium,” refer to media that are suitable to support the growth of cells in vitro (i.e., cell cultures, cell lines, etc.). It is not intended that the term be limited to any particular culture medium. For example, it is intended that the definition encompass maintenance media as well as other media for the differentiation or specialization of cells. Indeed, it is intended that the term encompass any culture medium suitable for the growth of the cell cultures and cells of interest.

As used herein, “tissue” means an aggregate of cells. In some embodiments, the cells in the tissue are cohered or fused.

As used herein, “scaffold” refers to synthetic scaffolds such as polymer scaffolds and porous hydrogels, non-synthetic scaffolds such as pre-formed extracellular matrix layers, dead cell layers, and decellularized tissues, and any other type of pre-formed scaffold that is integral to the physical structure of the engineered tissue and not able to be removed from the tissue without damage/destruction of said tissue. In further embodiments, decellularized tissue scaffolds include decellularized native tissues or decellularized cellular material generated by cultured cells in any manner; for example, cell layers that are allowed to die or are decellularized, leaving behind the extracellular matrix (ECM) they produced while living.

The terms “individual,” “subject,” “host,” and “patient,” are used interchangeably herein and refer to any mammalian subject for whom diagnosis, treatment, or therapy is desired, particularly humans.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

The terms “decrease”, “reduced”, “reduction”, “to a lesser extent,” or “inhibit” are all used herein to mean a decrease or lessening of a property, level, or other parameter by a statistically significant amount. In some embodiments, “reduced,” “reduction,” “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “increases”, or “enhance” or “activate” or “to a greater extent” are all used herein to generally mean an increase of a property, level, or other parameter by a statistically significant amount; for the avoidance of any doubt, the terms “increased”, “increase”, “to a greater extent,” “enhance” or “activate” can refer to an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

As used herein, “homogeneous” is intended to refer to a population of cells that have a uniform appearance and have uniform expression of the key markers indicative of that population. The term “homogeneous” is intended to encompass a population of cells that are predominantly of the same phenotype and can include up to 5% or up to 10% of cells of a different phenotype. As one example, a composition comprising a homogenous population of iPSC-derived chondrocytes may comprise 90%, 95% or more iPSC-derived chondrocytes. As another example, a composition comprising a homogenous population of iPSC-derived hypertrophic chondrocytes may comprise 90%, 95% or more iPSC-derived hypertrophic chondrocytes. As further example, a composition comprising a homogenous population of iPSC-derived articular chondrocytes may comprise 90%, 95% or more iPSC-derived articular chondrocytes.

As used herein, a “reference level” refers to the level of a marker or parameter in a normal, otherwise unaffected cell population or tissue (e.g., a cell, tissue, or biological sample obtained from a healthy subject, or a biological sample obtained from the subject at a prior time point, e.g., cell, tissue, or a biological sample obtained from a patient prior to being diagnosed with a disease, or a biological sample that has not been contacted with an agent or composition as disclosed herein). Alternatively, a reference level can also refer to the level of a given marker or parameter in a subject, organ, tissue, or cell, prior to administration of a treatment, e.g., with an agent or via administration of a transplant composition.

As used herein, a “control” or an “appropriate control” refers to an untreated, otherwise identical cell, subject, organism, or population (e.g., a cell, tissue, or biological sample that was not contacted by an agent or composition described herein) relative to a cell, tissue, biological sample, or population contacted or treated with a given treatment. For example, an appropriate control can be a cell, tissue, organ or subject that has not been contacted with an agent or administered a cell as described herein.

In one or more embodiments described herein, assessing the expression of various genes involves includes comparing the fold change. In one embodiment, the fold change is used to measure the change in the expression level of genes. In one embodiment the expression of a gene can be expressed as relative expression comparative to a housekeeping gene. In one embodiment, the fold change is measured by RPKM. As used herein, the term “RPKM” refers to “Reads Per Kilobase per Million mapped reads”. The term RPKM refers to a method of quantifying gene expression from RNA sequencing data by normalizing for total read length and the number of sequencing reads. In one embodiment, RPKM calculation provides a normalization for comparing gene coverage values. The RPKM value corrects for differences in both sample sequencing depth and gene length. In one example, the RPKM can be calculated via the following formula: numReads/(geneLength/1000*totalNumReads/1,000,000); wherein, “numReads” refers to the number of reads mapped to a gene sequence; wherein, “geneLength” refers to the length of the gene sequence; and wherein, “totalNumReads” refers to the total number of mapped reads of a sample.

The term “agonist” or “activator” may be used interchangeably and as used herein means an activator, for example, of a pathway or signaling molecule. An agonist of a molecule can retain substantially the same, or a subset, of the biological activities of the molecule (e.g. FGF). For example, an FGF agonist or “FGF activator” or “FGF pathway activator” means a molecule that selectively activates FGF signaling.

The term “inhibitor” as used herein means a selective inhibitor, for example of a pathway or signaling molecule. An inhibitor or antagonist of a molecule (e.g. BMP4 inhibitor) can inhibit one or more of the activities of the naturally occurring form of the molecule. For example, a BMP4 inhibitor is a molecule that selectively inhibits BMP4 signaling.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination. Any example or embodiment herein shall be taken to apply mutatis mutandis to any other example or embodiment unless specifically stated otherwise.

The present disclosure is not to be limited in scope by the specific examples described herein, which are intended for the purpose of exemplification only. Functionally-equivalent methods and systems are clearly within the scope of the disclosure, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The disclosure is hereinafter described by way of the following non-limiting Examples and with reference to the accompanying drawings. Although the examples herein concern humans and the language is primarily directed to human concerns, the concepts described herein are applicable to other animals. These and other aspects and features of the present disclosure will become more fully apparent from the following description and appended claims, or may be learned by the practice of the disclosure as set forth hereinafter.

A reference herein to a patent document or other matter which is given as prior art is not to be taken as an admission that that document or matter was known or that the information it contains was part of the common general knowledge as at the priority date of any of the claims.

### Chondrocyte Progenitors and Chondrocytes

Described herein are methods for generating chondrocytes, chondrocyte-like cells, cartilage-like tissue, cartilage, or a combination thereof from a population stem-cell derived sclerotome cells. Also described herein are the products of such methods. The cartilage or cartilage like tissue generated by the novel methods developed by the inventors yields a product which is phenotypically more accurate and of higher quality that that produced by methods described previously.

Cartilage ECM has a distinctive composition of collagen types and non-collagenous components. While all of these are important, it is the distribution of collagen types that provides the most distinctive signature of the cartilage tissue. The pattern of collagen expression, alone, would be widely accepted as the defining feature of cartilage tissues.

Articular cartilage displays the following pattern of collagen expression (% of total collagens): Collagen II: 90-95%-main fibrillar component, Collagen XI 3%-associated with collagen II fibrils, Collagen IX: 1%-associated with collagen II fibrils, Collagen X <1%, Collagen VI: 0-1%. Other proteoglycans, glycoproteins and proteins important in development and articular cartilage include Aggrecan (ACAN), Link protein (HAPLN1), Biglycan (BGN), Decorin (DCN), Fibromodulin (FBN), Chondroadherin (CHAD), CILP, COMP, Matrilin 1 (MATN1), Matrilin 3 (MATN3). In addition, articular cartilage specifically contains other ECM components including PRG4 and elastic fibre components ELN, LTBP2, TGFBI, MFAP4 and MFAP5.

Growth plate cartilage has many of the same ECM components as the articular cartilage but the proportions are different. Collagen composition: Growth plate cartilage has less collagen II (and associated collagens IX and XI) as a proportion of the total collagen. Collagen X is only expressed by growth plate chondrocytes and as such is the sentinel marker of this type of cartilage. It becomes the predominant collagen in the growth pate cartilage. Various cartilage non-collagenous protein components such as ACAN, MATN1 and COMP are reduced. Other non-collagenous proteins such as IBSP, MGP and SPP1 are upregulated.

The inventors have discovered that utilizing the novel protocols described herein, 3D aggregations of chondrocytes (chondrocyte organoids or “chondronoids”) can be produced which possess Collagen II (COL2A1) as a major component and smaller amounts of the collagen II-associated collagens IX and XI (COL9A1, COL9A2, COL9A3; COL11A1, COL11A2). The chondronoids possess an organized structure of this collagen II matrix and demonstrate the distinctive morphology of the chondrocyte and the collagen II fibrils. Importantly there is no collagen X or collagen I at this stage of development which would indicate either poor chondrocyte formation (collagen I) or hypertrophy (collagen X). Collagen X in particular is upregulated also in osteoarthritis cartilage-so having collagen X would be deleterious to use of this cartilage for cartilage repair. These findings demonstrate that the cartilage or cartilage-like material produced by these methods is of a high quality and phenotypically accurate.

Accordingly an aspect, of the present invention includes a method for generating chondrocytes, chondrocyte-like cells, cartilage-like tissue, cartilage, or a combination thereof, the method comprising:
a) culturing a three-dimensional aggregation of sclerotome cells with an FGF pathway activator to produce a three-dimensional aggregation of chondrocyte progenitor cells; and
b) culturing the three-dimensional aggregation of chondrocyte progenitor cells generated in step a) in the absence of said FGF pathway activator to produce a three-dimensional aggregation of chondrocytes, or chondrocyte-like cells, that express COL2A1 and ACAN;
wherein the method comprises transferring the three-dimensional aggregation of sclerotome cells or the three-dimensional aggregation of chondrocyte progenitor cells to orbital culture.

In an embodiment, the culture of three-dimensional aggregations in step a) and/or b) is performed in the absence of a TGF-beta antagonist and/or in the absence of a BMP pathway inhibitor. In another embodiment, the culture of three-dimensional aggregations in step a) is performed in the absence of a TGF-beta antagonist and/or in the absence of a BMP pathway inhibitor.

In an embodiment the culture of three-dimensional aggregations in steps a) and b) are non-adherent, low attachment or suspension culture.

In an embodiment, the culture of three-dimensional aggregations in step a) and b) is performed in pellet culture format. For clarity, step a) and b) are not performed in monolayer culture format.

In an embodiment, culture of three-dimensional aggregations in step b) is performed in an orbital rotary culture.

The inventors have discovered that culture of sclerotome with a pulse of an FGF pathway activator produces chondrocytes with more favorable ECM profile. In an embodiment, the period of culture in step a), in the presence of an FGF activator, is for a period of at least 7 days. In another embodiment, the period of culture in step a), in the presence of an FGF pathway activator, is for a period of from 7 to about 42 days. In another embodiment, the period of culture in step a), in the presence of an FGF pathway activator, is for a period of from 7 to about 21 days. In another embodiment, the period of culture in step a), in the presence of an FGF pathway activator, is for a period of about 10-17 days. In another embodiment, the period of culture in step a), in the presence of an FGF pathway activator, is for a period of about 14 days.

In the methods described herein, an agonist or activator, inhibitor or cell-culture component can be added on commencement of a time period for a specific time period or added repeatedly during a time period for example with media changes. For example, the FGF pathway activator may added with culture media replacement during the aforementioned time periods.

In embodiments of the various aspects and other embodiments described herein, the FGF pathway activator is selected from the group consisting of FGF2, FGF4, FGF9, FGF19, FGF21, FGF3, FGF5, FGF6, FGF8a, FGF16, FGF17, FGF18, FGF20 and FGF23. In a preferred embodiment the FGF pathway activator is FGF2.

In another embodiment of the various aspects and other embodiments described herein, the FGF pathway activator is present in an amount of from about 1 ng/ml-about 100 ng/ml. In another embodiment, the FGF pathway activator is present in an amount of from about 10 ng/ml-about 50 ng/ml. In another embodiment, the FGF pathway activator is present in an amount of about 20 ng/ml.

In an embodiment, the period of culture in step b), in the absence of an FGF pathway activator, is for a period of at least 7 days. In another embodiment, the period of culture in step b) is for a period of from about 7 to about 28 days. In another embodiment, the period of culture in step b) is for a period of from about 7 to about 21 days. In another embodiment, the period of culture in step b) is for a period of from about 10-17 days. In another embodiment, the period of culture in step b) is for a period of from about 14 days.

In an embodiment, the culture in step b) is performed in the absence of any growth factors,

In an embodiment, the 3D aggregation of chondrocyte progenitor cells are cultured, in step b) for a period of time sufficient to achieve expression of collagen II (COL2A1) and aggrecan (ACAN). In some embodiments, the 3D aggregation of chondrocyte progenitor cells are cultured, in step b) for a period of time sufficient to achieve expression of collagen II (COL2A1) and aggrecan (ACAN) at a ratio in the range of from 20:1 to 5:1 relative to each other. In some embodiments, the ratio of collagen II (COL2A1) to aggrecan (ACAN) is about 10:1.

In an embodiment, the 3D aggregation of chondrocyte progenitor cells are cultured in step b) for a period of time sufficient to achieve yield an increase in the relative expression of expression of collagen II (COL2A1) and aggrecan (ACAN) to a housekeeping gene of at least 10 fold and at least 20 fold, respectively. In some embodiments, the increase in the relative expression of collagen II (COL2A1) and aggrecan (ACAN) to a housekeeping gene is an increase of at least 40 fold and at least 100 fold, respectively. In one embodiment the housekeeper gene is GAPDH.

In another embodiment, COL1A1, which is lowly expressed relative the cartilage markers, COL2A1 and ACAN in chondroprogenitors does not increase in expression relative to GAPDH during the transition of the chondroprogenitors to chondrocytes. Accordingly, in another embodiment chondrocytes, chondrocyte-like cells, cartilage-like tissue, cartilage, or a combination thereof does not express collagen COL1A1 in levels greater than about 0.2% of the level of COL2A1.

In another embodiment, COL1A2, which is lowly expressed relative the cartilage markers, COL2A1 and ACAN in chondroprogenitors does not increase in expression relative to GAPDH during the transition of the chondroprogenitors to chondrocytes. Accordingly, in another embodiment chondrocytes, chondrocyte-like cells, cartilage-like tissue, cartilage, or a combination thereof does not express collagen COL1A2 in levels greater than about 0.2% of the level of COL2A1.

In another embodiment chondrocytes, chondrocyte-like cells, cartilage-like tissue, cartilage, or a combination thereof express one or more further genes selected from the group consisting of COL11A1, COL11A2, COL9A1, COL9A2, COL9A3, MATN1 and MATN3. In another embodiment chondrocytes, chondrocyte-like cells, cartilage-like tissue, cartilage, or a combination thereof express COL2A1, ACAN, COL11A1, COL11A2, COL9A1, COL9A2, COL9A3, MATN1 and MATN3.

In another embodiment chondrocytes, chondrocyte-like cells, cartilage-like tissue, cartilage, or a combination thereof does not express appreciable levels of collagen COL10A1.

In another embodiment, the 3D aggregation of chondrocyte progenitor cells are cultured in step b) for a period of time and under conditions sufficient to produce a 3D aggregation of chondrocytes, or chondrocyte-like cells, wherein the expression pattern of collagen in the 3D aggregation of chondrocytes, or chondrocyte-like cells is: about 30-50% COL2A1, about 5-40% each of COL11A1 and COL11A2, about 1-30% each of COL9A1, COL9A2, COL9A3. In another embodiment the expression pattern of collagen in the 3D aggregation of chondrocytes, or chondrocyte-like cells is about 40-50% COL2A1, about 10-30% each of COL11A1 and COL11A2, about 5-15% each of COL9A1, COL9A2, COL9A3. In another embodiment the expression pattern of collagen in the 3D aggregation of chondrocytes, or chondrocyte-like cells is about 40% to about 50% COL2A1, about 15% to about 25% COL11A1, about 5% to about 20% COL11A2, about 5% to about 30% COL9A1, about 1% to about 10% COL9A2, and about 1% to about 10% COL9A3.

Through detailed studies, described herein, the inventors have surprisingly found that transfer of 3D aggregates of chondrocyte progenitors to orbital rotary culture impacts the rate of maturation to chondrocytes, and in particular impacts the composition of the resulting chondronoids. That is orbital rotary culture affects differentiation rate and specificity. Differentiation of 3D aggregates of sclerotome cells cultured in static conditions versus rotary culture showed that rotary culture slowed chondrocyte development or delayed hypertrophy. Surprisingly, the inventors found that this impact on chondrocyte development can be overcome by altering the timing of transfer to rotary culture. As detailed in the Examples set forth herein delaying transfer to rotary culture was found to lead to more homogenous compositions of hypertrophic chondrocytes with noticeably enlarged morphology and collagen X expression, whereas early transfer to rotary culture or static culture was found to yield compositions comprising a larger number of non-cartilage cells. Accordingly, depending on the desired outcome the use of rotary culture and timing of such use can be utilized to alter the resulting product of culture.

Accordingly, in one embodiment, the method comprises transferring the 3D aggregation of sclerotome cells to orbital rotary culture. In another embodiment the method comprises transferring the 3D aggregation of sclerotome cells to orbital rotary culture from the commencement of step a) of the method, after 1 day, after 2 days, after 3 days, after 4 days, after 5 days, after 6 days, after 7 days, after 8 days, after 9 days, after 10 days, after 11 days, after 12 days, after 13 days, after 14 days, after 15 days, after 16 days, after 17 days, after 18 days, after 19 days, after 20 days, or after 21 days. In another embodiment, the method comprises delaying transferring the 3D aggregation of sclerotome cells to orbital rotary culture. In another embodiment the method comprises transferring the 3D aggregation of sclerotome cells to orbital rotary culture after 4 days, after 5 days, after 6 days, after 7 days, after 8 days, after 9 days, after 10 days, after 11 days, after 12 days, after 13 days, after 14 days, after 15 days, after 16 days, after 17 days, after 18 days, after 19 days, after 20 days, or after 21 days. In another embodiment the method comprises transferring the 3D aggregation of sclerotome cells to orbital rotary culture at any time from day 5 onwards, day 6 onwards or day 7 onwards. In another embodiment the method comprises transferring the 3D aggregation of sclerotome cells to orbital rotary culture at any time from day 7 to day 14. In a further embodiment, the method comprises transferring the 3D aggregation of chondrocyte progenitors (e.g. the product of step a)) to orbital rotary culture. In another embodiment the method comprises transferring the 3D aggregation of chondrocyte progenitors cells to orbital rotary culture from the commencement of step b) of the method, after 1 day, after 2 days, after 3 days, after 4 days, after 5 days, after 6 days, after 7 days, after 8 days, after 9 days, after 10 days, after 11 days, after 12 days, after 13 days, or after 14 days.

In one embodiment, the 3D aggregation of sclerotome cells is a high density aggregation of cells. In one embodiment, the 3D aggregation of cells is comprised of from about 10,000 cells to about 1,000,000 cells. In another embodiment the 3D aggregation of sclerotome cells is comprised of from about 5×10⁴ cells to about 5×10⁵ cells. In another embodiment, the 3D aggregation of sclerotome cells is comprised of from about 1×10⁵ cells to about 3×10⁵ cells. In another embodiment, the 3D aggregation of sclerotome cells is comprised of about 1×10⁵ to 5×10⁵, preferably about 2×10⁵ cells.

### Articular Chondrocytes and Articular Cartilage

In one embodiment the method is for generating articular (non-hypertrophic) chondrocytes or articular chondrocyte-like cells, articular cartilage-like tissue, articular cartilage, or a combination thereof, and step c) comprises culturing said three-dimensional aggregation of chondrocytes, or chondrocyte-like cells with a TGF-beta agonist to produce articular (non-hypertrophic) chondrocytes or articular chondrocyte-like cells, articular cartilage-like tissue, articular cartilage, or a combination thereof.

Another aspect, of the present invention includes a method for generating articular (non-hypertrophic) chondrocytes or articular chondrocyte-like cells, articular cartilage-like tissue, articular cartilage, or a combination thereof, the method comprising:
a) culturing a three-dimensional aggregation of sclerotome cells with an FGF pathway activator to produce a three-dimensional aggregation of chondrocyte progenitor cells;
b) culturing the three-dimensional aggregation of chondrocyte progenitor cells with an FGF pathway activator and a TGF-beta agonist to produce a three-dimensional aggregation of chondrocytes, or chondrocyte-like cells, that express COL2A1 and ACAN;
c) culturing said three-dimensional aggregation of chondrocytes, or chondrocyte-like cells with a TGF-beta agonist for an extended period of time to produce articular (non-hypertrophic) chondrocytes or articular chondrocyte-like cells, articular cartilage-like tissue, articular cartilage, or a combination thereof
wherein the method comprises transferring the three-dimensional aggregation of sclerotome cells, the three-dimensional aggregation of chondrocyte progenitor cells or the three-dimensional aggregation of chondrocytes or chondrocyte-like cells to orbital culture.

In an embodiment the method is for generating articular (non-hypertrophic) chondrocytes or articular chondrocyte-like cells, articular cartilage-like tissue, articular cartilage, or a combination thereof, and step c) comprises culturing said three-dimensional aggregation of chondrocytes, or chondrocyte-like cells with a TGF-beta agonist to produce articular (non-hypertrophic) chondrocytes or articular chondrocyte-like cells, articular cartilage-like tissue, articular cartilage, or a combination thereof that express PRG4.

In an embodiment, the TGF-beta agonist is selected from TGFb1, TGFb2, TGFb3 and/or combinations thereof. In an embodiment, the TGF-beta agonist is TGFb3.

In an embodiment the culturing said three-dimensional aggregation of chondrocytes, or chondrocyte-like cells with the TGF-beta agonist is for a period of at least 2 weeks. In an embodiment the culturing said three-dimensional aggregation of chondrocytes, or chondrocyte-like cells with the TGF-beta agonist is for a period selected from about 2 weeks to about 10 weeks. In an embodiment the culturing said three-dimensional aggregation of chondrocytes, or chondrocyte-like cells with the TGF-beta agonist is for a period selected from about 2 weeks to about 7 weeks. In an embodiment the culturing said three-dimensional aggregation of chondrocytes, or chondrocyte-like cells with the TGF-beta agonist is for a period selected from about 2 weeks, about 3 weeks, about 4 weeks, about 5 weeks, about 6 weeks and about 7 weeks.

In an embodiment the culturing said three-dimensional aggregation of chondrocytes, or chondrocyte-like cells with the TGF-beta agonist is for a period of about 5 weeks.

In an embodiment, the period of culture in step a), in the presence of an FGF pathway activator, is for a period of at least 2 to up to 21 days. In another embodiment, the period of culture in step a), in the presence of an FGF pathway activator, is for a period of from 2 to about 14 days. In another embodiment, the period of culture in step a), in the presence of an FGF pathway activator, is for a period of about 5-10 days. In another embodiment, the period of culture in step a), in the presence of an FGF pathway activator, is for a period of about 7 days.

In an embodiment, the period of culture in step b), in the presence of an FGF pathway activator and a TGF-beta agonist is for a period of at least 2 to up to 21 days. In another embodiment, the period of culture in step b), in the presence of an FGF pathway activator and a TGF-beta agonist, is for a period of from 2 to about 14 days. In another embodiment, the period of culture in step b), in the presence of an FGF pathway activator and a TGF-beta agonist, is for a period of about 5-10 days. In another embodiment, the period of culture in step b), in the presence of an FGF pathway activator and a TGF-beta agonist is for a period of about 7 days.

In an embodiment, the period of culture in step c), in the presence of a TGF-beta agonist is for a period of at least 2 to up to 42 days. In another embodiment, the period of culture in step c), in the presence of a TGF-beta agonist, is for a period of from 14 to about 35 days. In another embodiment, the period of culture in step c), in the presence of a TGF-beta agonist, is for a period of about 21 to about 35 days. In another embodiment, the period of culture in step c), in the presence of a TGF-beta agonist is for a period of about 28 days.

In an embodiment, the culture of three-dimensional aggregations in step a) and/or step b) is performed in the absence of a TGF-beta antagonist and/or in the absence of a BMP pathway inhibitor.

In an embodiment, the culture of three-dimensional aggregations in step a), step b) and step c) are performed in pellet culture format. For clarity, step a), step b) and step c) are not performed in monolayer culture format.

In an embodiment, culture of three-dimensional aggregations in step c) is performed in an orbital rotary culture.

In another embodiment, the articular chondrocytes or articular chondrocyte-like cells, articular cartilage-like tissue, articular cartilage, or a combination thereof expresses COL2A1, ACAN and PRG4, and optionally, one or more markers selected from ASPN, CILP, CILP2, EMILIN1, EMILIN3, FBLN1, and FBLN3.

In another embodiment, the aggregation of chondrocytes, or chondrocyte-like cells are cultured with TGF-beta3 for a period of time and under conditions sufficient to produce a population of articular chondrocytes or articular chondrocyte-like cells, articular cartilage-like tissue, articular cartilage, or a combination thereof, wherein the expression pattern of collagen in the articular chondrocytes or articular chondrocyte-like cells, articular cartilage-like tissue, articular cartilage, or a combination thereof is: about 70-95% collagen 2 (COL2A1) and about 1% to about 30% each of COL9A1 and COL9A2. In some embodiments the expression pattern of collagen in the articular chondrocytes or articular chondrocyte-like cells, articular cartilage-like tissue, articular cartilage, or a combination thereof is: about 80-95% collagen 2 (COL2A1) and about 1% to about 10% each of COL9A1 and COL9A2. In some embodiments, the collagen 2 (COL2A1) represents about 85% to about 95% of the total collagen. In some embodiments, collagen 2 (COL2A1) represents about 85% to about 95% and COL9A1 and COL9A2 together represents about 5% to about 15% of the total collagen.

### Hypertrophic Chondrocytes and Hypertrophic Cartilage

In an embodiment the method is for generating hypertrophic chondrocytes or hypertrophic chondrocyte-like cells, hypertrophic cartilage-like tissue, hypertrophic cartilage, or a combination thereof, the method comprising: a) culturing a three-dimensional aggregation of sclerotome cells with an FGF pathway activator to produce a three-dimensional aggregation of chondrocyte progenitor cells; and b) culturing the three-dimensional aggregation of chondrocyte progenitor cells generated in step a) in the absence of said FGF pathway activator to produce a three-dimensional aggregation of chondrocytes, or chondrocyte-like cells, that express COL2A1 and ACAN; and c) comprising culturing said three-dimensional aggregation of chondrocytes, or chondrocyte-like cells with triiodothyronine (T3) to produce hypertrophic chondrocytes, hypertrophic chondrocyte-like cells, hypertrophic cartilage-like tissue, hypertrophic cartilage, or a combination thereof that expresses COL10A1, wherein the method comprises transferring the three-dimensional aggregation of sclerotome cells or the three-dimensional aggregation of chondrocyte progenitor cells to orbital culture.

In an embodiment the culturing of said three-dimensional aggregation of chondrocytes, or chondrocyte-like cells with T3 is for an extended period of time. In some embodiments the culturing of said three-dimensional aggregation of chondrocytes, or chondrocyte-like cells with T3 is for a period ranging from about 2 days to about 4 weeks. In some embodiments the culturing of said three-dimensional aggregation of chondrocytes, or chondrocyte-like cells with T3 is for a period of at least 1 week. In some embodiments the culturing of said three-dimensional aggregation of chondrocytes, or chondrocyte-like cells with T3 is for a period of at least 2 weeks. In an embodiment the culturing said three-dimensional aggregation of chondrocytes, or chondrocyte-like cells with T3 is for a period selected from about 1 week, about 2 weeks, about 3 weeks, or about 4 weeks. In some embodiments the culturing of said three-dimensional aggregation of chondrocytes, or chondrocyte-like cells with T3 is for a period of about 1 week up to about 4 weeks. In an embodiment the culturing said three-dimensional aggregation of chondrocytes, or chondrocyte-like cells with T3 is for a period of from about 2 weeks to about 4 weeks.

In an embodiment, the period of culture in step a), in the presence of an FGF pathway activator, is for a period of at least 7 days. In another embodiment, the period of culture in step a), in the presence of an FGF pathway activator, is for a period of from 7 to about 42 days. In another embodiment, the period of culture in step a), in the presence of an FGF pathway activator, is for a period of from 7 to about 21 days. In another embodiment, the period of culture in step a), in the presence of an FGF pathway activator, is for a period of about 10-17 days. In another embodiment, the period of culture in step a), in the presence of an FGF pathway activator, is for a period of about 14 days.

In an embodiment, the period of culture in step b), in the absence of an FGF pathway activator, is for a period of at least 7 days. In another embodiment, the period of culture in step b) is for a period of from about 7 to about 42 days. In another embodiment, the period of culture in step b) is for a period of from about 7 to about 28 days. In another embodiment, the period of culture in step b) is for a period of from about 7 to about 21 days. In another embodiment, the period of culture in step b) is for a period of from about 10-17 days. In another embodiment, the period of culture in step b) is for a period of from about 14 days.

In an embodiment, the culture in step b) is performed in the absence of any growth factors.

In an embodiment, the culture of three-dimensional aggregations in step a), step b) and/or step c) is performed in the absence of a TGF-beta antagonist and/or in the absence of a BMP pathway inhibitor. In an embodiment, the culture of three-dimensional aggregations in step a) is performed in the absence of a TGF-beta antagonist and/or in the absence of a BMP pathway inhibitor

In an embodiment, the culture of three-dimensional aggregations in step a), step b) and/or step c) is performed in the absence of a BMP pathway activator and/or a Wnt pathway activator. In an embodiment, the culture of three-dimensional aggregations in step c) is performed in the absence of a BMP pathway activator and/or a Wnt pathway activator.

In an embodiment, the culture of three-dimensional aggregations in steps a), b) and c) are performed in pellet culture format. For clarity, steps a), b) and c) are not performed in monolayer culture format.

In an embodiment, culture of three-dimensional aggregations in steps b) and c) are performed in an orbital rotary culture.

In another embodiment, the hypertrophic chondrocytes, hypertrophic chondrocyte-like cells, hypertrophic cartilage-like tissue, hypertrophic cartilage, or a combination thereof expresses COL2A1 and COL10A1. In another embodiment, the hypertrophic chondrocytes, hypertrophic chondrocyte-like cells, hypertrophic cartilage-like tissue, hypertrophic cartilage, or a combination thereof expresses COL2A1 and COL10A1 at a ratio in the range of about 1:1 to about 2.5:1.

In another embodiment, the aggregation of chondrocytes, or chondrocyte-like cells are cultured with T3 for a period of time and under conditions sufficient to produce a population of hypertrophic chondrocytes, hypertrophic chondrocyte-like cells, hypertrophic cartilage-like tissue, hypertrophic cartilage, or a combination thereof, wherein the expression pattern of collagen in the hypertrophic chondrocytes, hypertrophic chondrocyte-like cells, hypertrophic cartilage-like tissue, hypertrophic cartilage, or a combination thereof is: about 30-50% collagen 2 (COL2A1) and about 30-50% COL10A1 and about 1% to about 10% each of COL9A1 and COL9A2. In some embodiments the expression pattern of collagen in the hypertrophic chondrocytes, hypertrophic chondrocyte-like cells, hypertrophic cartilage-like tissue, hypertrophic cartilage, or a combination thereof is: about 40-50% collagen 2 (COL2A1) and about 40-50% COL10A1 and about 1% to about 5% each of COL9A1 and COL9A2. In some embodiments, the collagen 2 (COL2A1) and COL10A1 represents about 90% to about 99% of the total collagen. In some embodiments, collagen 2 (COL2A1) and COL10A1 represents about 95% to about 99% and COL9A1 and COL9A2 represents about 1% to about 5% of the total collagen.

### Osteoblasts and Bone-Organoid Formation

Through detailed studies described herein, the inventors have discovered that chondrocytes that mature to hypertrophic chondrocytes prepared according to the foregoing methods and can transition to osteoblasts and produce a mineralized extracellular matrix. This can occur in vivo through transplantation of the hypertrophic chondrocytes, or in vitro.

Accordingly, in another embodiment there is provided a method of producing a population of osteoblasts or a bone-like organoid, the method comprising:
a) producing hypertrophic chondrocytes, hypertrophic chondrocyte-like cells, cartilage-like tissue, cartilage, according to the methods described herein; and
b) culturing the hypertrophic chondrocytes, hypertrophic chondrocyte-like cells, hypertrophic cartilage-like tissue, or hypertrophic cartilage with an osteogenic culture medium to generate osteoblasts or a bone-like organoid that expresses COL1A1 and COL1A2.

In one embodiment culturing the hypertrophic chondrocytes, hypertrophic chondrocyte-like cells, hypertrophic cartilage-like tissue is in orbital rotary culture. In another embodiment the culture is for an extended time period. In another embodiment the culture is for a period of up to about 3 weeks.

In one embodiment, the osteogenic differentiation culture medium comprises β-glycerophosphate, ascorbic acid 2-phosphate, sodium ascorbate, and dexamethasone. In another embodiment, the osteogenic differentiation culture medium further comprises a WNT pathway activator for about the first 3-7 days of culture. In another embodiment, the WNT pathway activator is selected from the group consisting of CHIR99021 (6-[[2-[[4-(2,4-Dichlorophenyl)-5-(5-methyl-1H-imidazol-2-yl)-2-pyrimidinyl]amino]ethyl]amino]-3-pyridinecarbonitrile), Wnt-1, Wnt-2, Wnt-2b, Wnt-3a, Wnt-4, Wnt-5a, Wnt-5b, Wnt-6, Wnt-7a, Wnt-7a/b, Wnt-7b, Wnt-8a, Wnt-8b, Wnt-9a, Wnt-9b, Wnt-10a, Wnt-10b, Wnt-11, Wnt-16b, RSPO co-agonists, lithium chloride, TDZD8 (4-Benzyl-2-methyl-1,2,4-thiadiazolidine-3,5-dione), BIO-Acetoxime ((2′Z,3′E)-6-Bromoindirubin-3′-acetoxime), A1070722 (1-(7-Methoxyquinolin-4-yl)-3-[6-(trifluoromethyl)pyridin-2-yl]urea), HLY78 (4-Ethyl-5,6-Dihydro-5-methyl-[1,3]dioxolo[4,5-j]phenanthridine), CID 11210285 hydrochloride (2-Amino-4-(3,4-(methylenedioxy)benzylamino)-6-(3-methoxyphenyl)pyrimidine hydrochloride), WAY-316606, (hetero) arylpyrimidines, IQ1, QS11, SB-216763, and DCA. In a preferred embodiment the Wnt pathway activator is CHIR99021.

In one embodiment, the osteogenic differentiation culture medium does not contain a BMP pathway activator, IL1A, IL1B, IL6 and/or TNF.

### Sclerotome

Methods to generate sclerotome, going from pluripotent stem cells to primitive streak, paraxial mesoderm, early somites, then sclerotome have been described previously (see eg. Loh et al., 2016). The method of the present invention can employ sclerotome cells produced by any known method.

In one embodiment the sclerotome cells employed in the methods described herein are obtained by a method comprising: i) culturing a population of pluripotent progenitor cells with a composition comprising a TGF-beta pathway activator, a Wnt pathway activator, an FGF pathway activator, and a P13K inhibitor for a time period of about 24 hours; ii) culturing the cells of step i) with a composition comprising a TGF-beta pathway inhibitor, a Wat pathway activator, an FGF pathway activator, and a BMP pathway inhibitor for a time period of about 24 hours; iii) culturing the cells of step ii) with a composition comprising a Wat pathway inhibitor, a BMP pathway inhibitor and a MEK/ERK pathway inhibitor for a time period of about 24 hours; and iv) contacting the cells of step iii) with a composition comprising a Wnt pathway inhibitor and a Hedgehog pathway activator, for a time period of about 72 hours to generate sclerotome cells.

In one embodiment the TGF-beta pathway activator is selected from the group consisting of Activin A, TGF-beta1, TGF-beta2, TGF-beta3, IDE1/2 (IDE1 (1-[2-[(2-Carboxyphenyl)methylene]hydrazide]heptanoic acid), IDE2 (Heptanedioic acid-1-(2-cyclopentylidenehydrazide)), and Nodal. In a preferred embodiment the TGF-beta pathway activator is Activin A.

In an embodiment, the composition of step iii) used to culture the cells of step ii) does not include a TGF-beta pathway activator and/or an FGF pathway activator.

In an embodiment, the composition of step iv) that is used to culture the cells of step iii) does not include a TGF-beta pathway inhibitor, an FGF pathway activator and/or a BMP pathway inhibitor.

In one embodiment the Wnt pathway activator is selected from the group consisting of CHIR99021 (6-[[2-[[4-(2,4-Dichlorophenyl)-5-(5-methyl-1H-imidazol-2-yl)-2-pyrimidinyl]amino]ethyl]amino]-3-pyridinecarbonitrile), Wnt-1, Wnt-2, Wnt-2b, Wnt-3a, Wnt-4, Wnt-5a, Wnt-5b, Wnt-6, Wnt-7a, Wnt-7a/b, Wnt-7b, Wnt-8a, Wnt-8b, Wnt-9a, Wnt-9b, Wnt-10a, Wnt-10b, Wnt-11, Wnt-16b, RSPO co-agonists, lithium chloride, TDZD8 (4-Benzyl-2-methyl-1,2,4-thiadiazolidine-3,5-dione), BIO-Acetoxime ((2′Z,3′E)-6-Bromoindirubin-3′-acetoxime), A1070722 (1-(7-Methoxyquinolin-4-yl)-3-[6-(trifluoromethyl)pyridin-2-yl]urea), HLY78 (4-Ethyl-5,6-Dihydro-5-methyl-[1,3]dioxolo[4,5-j]phenanthridine), CID 11210285 hydrochloride (2-Amino-4-(3,4-(methylenedioxy)benzylamino)-6-(3-methoxyphenyl)pyrimidine hydrochloride), WAY-316606, (hetero) arylpyrimidines, IQ1, QS11, SB-216763, and DCA. In a preferred embodiment the Wnt pathway activator is CHIR99021.

In one embodiment the PI3K pathway inhibitor is selected from the group consisting of AS 252424 (5-[[5-(4-Fluoro-2-hydroxyphenyl)-2-furanyl]methylene]-2,4-thiazolidinedione), AS 605240 (5-(6-Quinoxalinylmethylene)-2,4-thiazolidine-2,4-dione), AZD 6482 ((−)-2-[[(1R)-1-[7-Methyl-2-(4-morpholinyl)-4-oxo-4H-pyrido[1,2-a]pyrimidin-9-yl]ethyl]amino]benzoic acid), BAG 956 (α,α,-Dimethyl-4-[2-methyl-8-[2-(3-pyridinyl) ethynyl]-1H-imidazo[4,5-c]quinolin-1-yl]-benzeneacetonitrile), CZC 24832 (5-(2-Amino-8-fluoro[1,2,4]triazolo[1,5-a]pyridin-6-yl)-N-(1,1-dimethylethyl)-3-pyridinesulfonamide), GSK 1059615 (5-[[4-(4-Pyridinyl)-6-quinolinyl]methylene]-2,4-thiazolidenedione), KU 0060648 (4-Ethyl-N-[4-[2-(4-morpholinyl)-4-oxo-4H-1-benzopyran-8-yl]-1-dibenzothienyl]-1-piperazineacetamide), LY 294002 hydrochloride (2-(4-Morpholinyl)-8-phenyl-4H-1-benzopyran-4-one hydrochloride), 3-Methyladenine (3-Methyl-3H-purin-6-amine), PF 04691502 (2-Amino-8-[trans-4-(2-hydroxyethoxy)cyclohexyl]-6-(6-methoxy-3-pyridinyl)-4-methyl-pyrido[2,3-d]pyrimidin-7 (8H)-one), PF 05212384 (N-[4-[[4-(Dimethylamino)-1-piperidinyl]carbonyl]phenyl]-N′-[4-(4,6-di-4-morpholinyl-1,3,5-triazin-2-yl)phenyl]urea), PI 103 hydrochloride (3-[4-(4-Morpholinylpyrido[3′,2′: 4,5]furo[3,2-d]pyrimidin-2-yl]phenol hydrochloride), PI 828 (2-(4-Morpholinyl)-8-(4-aminophenyl)-4H-1-benzopyran-4-one), PP 121 (1-Cyclopentyl-3-(1H-pyrrolo[2,3-b]pyridin-5-yl)-1H-pyrazolo[3,4-d]pyrimidin-4-amine), Quercetin, TG 100713 (3-(2,4-Diamino-6-pteridinyl)-phenol), Wortmannin, PIK90, and GDC-0941. In a preferred embodiment the PI3K pathway inhibitor is PIK90.

In one embodiment the TGF-beta pathway inhibitor is selected from the group consisting of A-83-01 (3-(6-Methyl-2-pyridinyl)-N-phenyl-4-(4-quinolinyl)-1H-pyrazole-1-carbothioamide), D4476 (4-[4-(2,3-Dihydro-1,4-benzodioxin-6-yl)-5-(2-pyridinyl)-1H-imidazol-2-yl]benzamide), GW 788388 (4-[4-[3-(2-Pyridinyl)-1H-pyrazol-4-yl]-2-pyridinyl]-N-(tetrahydro-2H-pyran-4-yl)-benzamide), LY 364947 (4-[3-(2-Pyridinyl)-1H-pyrazol-4-yl]-quinoline), RepSox (2-(3-(6-Methylpyridine-2-yl)-1H-pyrazol-4-yl)-1,5-naphthyridine), SB431542 (4-[4-(1,3-benzodioxol-5-yl)-5-(2-pyridinyl)-1H-imidazol-2-yl]benzamide), SB-505124 (2-[4-(1,3-Benzodioxol-5-yl)-2-(1,1-dimethylethyl)-1H-imidazol-5-yl]-6-methyl-pyridine), SB 525334 (6-[2-(1,1-Dimethylethyl)-5-(6-methyl-2-pyridinyl)-1H-imidazol-4-yl]quinoxaline), SD208 (2-(5-Chloro-2-fluorophenyl)-4-[(4-pyridyl)amino]pteridine), ITD1 (4-[1,1′-Biphenyl]-4-yl-1,4,5,6,7,8-hexahydro-2,7,7-trimethyl-5-oxo-3-quinolinecarboxylic acid ethyl ester), DAN/Fc, antibodies to TGF-beta and TGF-beta receptors, TGF-beta inhibitory nucleic acids. In a preferred embodiment the TGF-beta pathway inhibitor is A-83-01.

In one embodiment the BMP pathway inhibitor is selected from the group consisting of Chordin, soluble BMPR1a, soluble BMPR1b, Noggin, LDN-193189, and Dorsomorphin. In a preferred embodiment the BMP pathway inhibitor is LDN-193189.

In one embodiment the Wnt pathway inhibitor is selected from the group consisting of C59 (4-(2-Methyl-4-pyridinyl)-N-[4-(3-pyridinyl)phenyl]benzeneacetamide), DKK1, IWP-2 (N-(6-Methyl-2-benzothiazolyl)-2-[(3,4,6,7-tetrahydro-4-oxo-3-phenylthieno[3,2-d]pyrimidin-2-yl)thio]-acetamide), Ant1.4Br, Ant 1.4CI, Niclosamide, apicularen, bafilomycin, XAV939 (3,5,7,8-Tetrahydro-2-[4-(trifluoromethyl)phenyl]-4H-thiopyrano[4,3-d]pyrimidin-4-one), IWR-1 (4-(1,3,3a,4,7,7a-Hexahydro-1,3-dioxo-4,7-methano-2H-isoindol-2-yl)-N-8-quinolinyl-Benzamide), NSC668036 (N-[(1,1-Dimethylethoxy) carbonyl]-L-alanyl-(2S)-2-hydroxy-3-methylbutanoyl-L-Alanine-(1S)-1-carboxy-2-methylpropyl ester hydrate), 2,4-diamino-quinazoline, Quercetin, ICG-001 ((6S,9aS)-Hexahydro-6-[(4-hydroxyphenyl)methyl]-8-(1-naphthalenylmethyl)-4,7-dioxo-N-(phenylmethyl)-2H-pyrazino[1,2-a]pyrimidine-1 (6H)-carboxamide), PKF115-584, BML-284 (2-Amino-4-[3,4-(methylenedioxy)benzylamino]-6-(3-methoxyphenyl)pyrimidine), FH-535, iCRT-14, JW-55, JW-67, antibodies to Wnts and Wnt receptors, and Wnt inhibitory nucleic acids. In a preferred embodiment the Wnt pathway inhibitor is C59.

In one embodiment the MEK/ERK pathway inhibitor is selected from the group consisting of AP 24534 (3-(2-Imidazo[1,2-b]pyridazin-3-ylethynyl)-4-methyl-N-[4-[(4-methyl-1-piperazinyl)methyl]-3-(trifluoromethyl)phenyl]-benzamide), PD173074 (N-[2-[[4-(Diethylamino)butyl]amino]-6-(3,5-dimethoxyphenyl)pyrido[2,3-d]pyrimidin-7-yl]-N′-(1,1-dimethylethyl) urea), FIIN 1 hydrochloride (N-(3-((3-(2,6-dichloro-3,5-dimethoxyphenyl)-7-(4-(diethylamino)butylamino)-2-oxo-3,4-dihydropyrimido[4,5-d]pyrimidin-1 (2H)-yl)methyl)phenyl) acrylamide), PD 161570 (N-[6-(2,6-Dichlorophenyl)-2-[[4-(diethylamino)butyl]amino]pyrido[2,3-d]pyrimidin-7-yl]-N′-(1,1-dimethylethyl) urea), SU 5402 (2-[(1,2-Dihydro-2-oxo-3H-indol-3-ylidene)methyl]-4-methyl-1H-pyrrole-3-propanoic acid), SU 6668 (5-[1,2-Dihydro-2-oxo-3H-indol-3-ylidene)methyl]-2,4-dimethyl-1H-pyrrole-3-propanoic acid), PD0325901 (N-[(2R)-2,3-Dihydroxypropoxy]-3,4-difluoro-2-[(2-fluoro-4-iodophenyl)amino]-benzamide), BIX 02189 ((3Z)-3-[[[3-[(Dimethylamino)methyl]phenyl]amino]phenylmethylene]-2,3-dihydro-N,N-dimethyl-2-oxo-1H-indole-6-carboxamide), FR 180204 (5-(2-Phenyl-pyrazolo[1,5-a]pyridin-3-yl)-1H-pyrazolo[3,4-c]pyridazin-3-ylamine), Pluripotin (N-[3-[7-[(1,3-Dimethyl-1H-pyrazol-5-yl)amino]-1,4-dihydro-1-methyl-2-oxopyrimido[4,5-d]pyrimidin-3 (2H)-yl]-4-methylphenyl]-3-(trifluoromethyl)benzamide), TCS ERK 11e (4-[2-[(2-Chloro-4-fluorophenyl)amino]-5-methyl-4-pyrimidinyl]-N-[(1S)-1-(3-chlorophenyl)-2-hydroxyethyl]-1H-pyrrole-2-carboxamide), TMCB (2-(4,5,6,7-Tetrabromo-2-(dimethylamino)-1H-benzo[d]imidazol-1-yl) acetic acid), XMD 8-92 (2-[[2-Ethoxy-4-(4-hydroxy-1-piperidinyl)phenyl]amino]-5,11-dihydro-5,11-dimethyl-6H-pyrimido[4,5-b][1,4]benzodiazepin-6-one), SU5402, AZD4547, BGJ398, AL 8697, AMG 548, CMPD-1, DBM 1285 dihydrochloride, EO 1428, JX 401, ML 3403, RWJ 67657, SB 202190, SB-203580, SB 239063, SB 706504, Scio-469, SKF 86002 dihydrochloride, SX 011, TA 01 (4-(2-(2,6-Difluorophenyl)-4-(fluorophenyl)-1H-imidazol-5-yl)pyridine), TA 02 (4-(2-(2-Fluorophenyl)-4-(fluorophenyl)-1H-imidazol-5-yl)pyridine), TAK 715, VX-702, and VX-745. In a preferred embodiment the MEK/ERK pathway inhibitor is PD0325901.

In one embodiment the Hedgehog pathway activator is selected from the group consisting of Hedgehog family ligands (Hh, Shh, Ihh, Dhh, etc.) and fragments thereof, benzothiophene smoothened agonists, SAG (Hh-Ag1.3), SAG21k (3-chloro-4,7-difluoro-N-(4-methoxy-3-(pyridin-4-yl)benzyl)-N-((1r,4r)-4-(methylamino)cyclohexyl)benzo[b]thiophene-2-carboxamide), Hh-Ag1.1, Hh-Ag1.5, and purmorphamine. In a preferred embodiment the Hedgehog pathway activator is purmorphamine.

In another embodiment said sclerotome cells express any one of PAX1, SOX9, FOXC2, PAX9, NKX3.2/BAPX1 and TWIST1.

In another embodiment, sclerotome cells are converted to 3D aggregates (high density pellet) after 4 days and maintained as such in culture thereafter. In another embodiment, cells at the sclerotome stage before pelleting can be frozen in medium containing 10% DMSO, stored under liquid nitrogen and retrieved for subsequent use in the methods described herein.

iPSCs

Any pluripotent stem cell population, including a human embryonic stem cell population (hESC) or an induced pluripotent stem cell population (iPSCs), can be used as the starting material to derive sclerotome cells to be used in the methods of the invention (e.g. to obtain iPSC-derived chondrocytes, chondrocyte-like cells, cartilage-like tissue, cartilage). In a preferred embodiment, population of pluripotent progenitor cells is a human iPSC population. Through detailed studies described herein, the inventors have discovered that key chondrocyte genes, SOX9, COL2A1, COL9A1, COL11A1 and ACAN were more highly expressed iPSC cells grown in feeder-free conditions and can differentiate more efficiently. Accordingly, in a further preferred embodiment the population of pluripotent progenitor cells (employed to generate sclerotome), are iPSCs are derived from feeder-free cell culture.

In another embodiment, cells obtained from a subject can be subjected to methods to generate patient specific iPSCs which can then be differentiated using the methods described herein. There are more than 430 genetic disorders of the skeleton caused by mutations in more than 360 different genes. Accordingly, in one embodiment the iPSCs used in the methods described herein are obtained from a patient having a disease selected from one of the following cartilage and bone disorders groups: FGFR3 chondrodysplasia group, Type 2 collagen group, Type 11 collagen group, Sulphation disorders group, Perlecan group, Aggrecan group, Filamin group and related disorders, TRPV4 group, Ciliopathies with major skeletal involvement, Multiple epiphyseal dysplasia and pseudoachondroplasia group, Metaphyseal dysplasias, Spondylometaphyseal dysplasias (SMD), Spondylo-epi-(meta)-physeal dysplasias (SE (M) D), Severe spondylodysplastic dysplasias, Acromelic dysplasias, Acromesomelic dysplasias, Mesomelic and rhizo-mesomelic dysplasias, Campomelic dysplasia and related disorders, Slender bone dysplasia group, Chondrodysplasia punctata (CDP) group, Neonatal osteosclerotic dysplasias, Osteopetrosis and related disorders, Osteogenesis imperfecta and decreased bone density group, Abnormal mineralization group, Lysosomal Storage Diseases with Skeletal Involvement (Dysostosis Multiplex group), Disorganized development of skeletal components group, Cleidocranial dysplasia and related disorders, Dysostoses with predominant craniofacial involvement.

### Compositions

Also provided in another embodiment is an isolated population of chondrocytes or chondrocyte-like cells, or cartilage-like tissue or cartilage, or a combination thereof produced according to the methods a method described herein. In one embodiment, the isolated population of chondrocytes or chondrocyte-like cells, or cartilage-like tissue or cartilage, or a combination thereof may be provided as a composition which optionally includes a suitable carrier.

In one embodiment there is provided a composition comprising a homogenous population of iPSC-derived chondrocytes, wherein said cells express collagen 2 (COL2A1) and ACAN at a ratio in the range of 20:1 to 5:1, preferably about 10:1 and does not substantially express COL10A1. In some embodiments, the homogenous population of iPSC-derived chondrocytes may also express COL9A1, COL9A2, COL9A3, COL11A1 and COL11A2. In some embodiments, COL2A1 represents about 30% to about 50% of the collagen expressed when determined by RNAseq analysis and expressed as the % of the total collagen RPKM. In some embodiments, COL2A1 represents about 30% to about 50%, COL11A1 represents about 10% to about 30%, COL11A2 represents about 5% to about 20%, COL9A1 represents about 5% to about 30%, COL9A2 represents about 1% to about 10% and COL9A3 represents about 1% to about 10% of the total collagen. In some embodiments, COL2A1 represents about 40% to about 50%, COL11A1 and COL11A2 each represent about 5% to about 40% and COL9A1, COL9A2 and COL9A3 each represent about 5% to about 15% of the total collagen. In another embodiment, COL2A1 represents about 45%, COL11A1 represents about 21%, COL11A2 represents about 10%, COL9A1 represents about 13%, COL9A2 represents about 6% and COL9A3 represents about 4% of the total collagen. Compositions of these embodiments may be suitable for repair or replacement of articular cartilage. Accordingly, in another embodiment there is provided a composition according to the foregoing embodiments, for use in the repair or replacement of articular cartilage in a subject in need thereof. In another embodiment there is provided a method of repairing or replacing articular cartilage in a subject in need thereof comprising administering to the subject a therapeutically effective amount of a composition according to the foregoing embodiments. In another embodiment there is provided use of a composition according to the foregoing embodiments the manufacture of a medicament for repairing or replacing articular cartilage in a subject in need thereof.

In another embodiment there is provided a composition comprising a homogenous population of iPSC-derived articular chondrocytes, wherein said cells express COL2A1, ACAN and PRG4, and optionally, one or more markers selected from ASPN, CILP, CILP2, EMILIN1, EMILIN3, FBLN1 and FBLN3. In some embodiments, the homogenous population of iPSC-derived articular chondrocytes express COL2A1, COL9A1 and COL9A2. In some embodiments, collagen 2 (COL2A1) represents about 70% to about 95% of the collagen expressed when determined by RNAseq analysis and expressed as the % of the total collagen RPKM. In some embodiments, collagen 2 (COL2A1) represents about 70% to about 95% and COL9A1 and COL9A2 each represent about 1% to about 30% of the total collagen. In some embodiments, collagen 2 (COL2A1) represents about 80% to about 95% and COL9A1 and COL9A2 each represent about 1% to about 10% of the total collagen. In some embodiments, collagen 2 (COL2A1) represents about 85% to about 95% and COL9A1 and COL9A2 each represent about 5% to about 15% of the total collagen. Compositions of these embodiments may be suitable for repair or replacement of articular cartilage. Accordingly, in another embodiment there is provided a composition according to the foregoing embodiments, for use in the repair or replacement of articular cartilage in a subject in need thereof. In another embodiment there is provided a method of repairing or replacing articular cartilage in a subject in need thereof comprising administering to the subject a therapeutically effective amount of a composition according to the foregoing embodiments. In another embodiment there is provided use of a composition according to the foregoing embodiments the manufacture of a medicament for repairing or replacing articular cartilage in a subject in need thereof.

In another embodiment there is provided a composition comprising a homogenous population of iPSC-derived hypertrophic chondrocytes, wherein said cells express collagen 2 (COL2A1) and COL10A1 at a ratio in the range of 1:2.5 to about 2.5:1. In some embodiments, the ratio of collagen 2 (COL2A1) to COL10A1 is in the range of about 1.5:1 to 1:1.5. In some embodiments, the homogenous population of iPSC-derived hypertrophic chondrocytes may also express COL9A1 and COL9A2. In some embodiments, collagen 2 (COL2A1) and COL10A1 represents about 90% to about 99% of the collagen expressed when determined by RNAseq analysis and expressed as the % of the total collagen RPKM. In some embodiments, collagen 2 (COL2A1) and COL10A1 represents about 90% to about 99% and COL9A1 and COL9A2 represents about 1% to about 10% of the total collagen. In some embodiments, collagen 2 (COL2A1) and COL10A1 represents about 95% to about 99% and COL9A1 and COL9A2 represents about 1% to about 5% of the total collagen. In some embodiments, collagen 2 (COL2A1) represents about 30% to about 50%, COL10A1 represents about 30% to about 50% and COL9A1 and COL9A2 each represent about 1% to about 10% of the total collagen. In some embodiments, collagen 2 (COL2A1) represents about 40% to about 50%, COL10A1 represents about 40% to about 50% and COL9A1 and COL9A2 each represent about 1% to about 5% of the total collagen. Compositions of these embodiments may be suitable for repair or replacement of hypertrophic cartilage. Accordingly, in another embodiment there is provided a composition according to the foregoing embodiments, for use in the repair or replacement of hypertrophic cartilage in a subject in need thereof. In another embodiment there is provided a method of repairing or replacing hypertrophic cartilage in a subject in need thereof comprising administering to the subject a therapeutically effective amount of a composition according to the foregoing embodiments. In another embodiment there is provided use of a composition according to the foregoing embodiments the manufacture of a medicament for repairing or replacing hypertrophic cartilage in a subject in need thereof.

Also provided in another embodiment is an isolated population of osteoblasts or a bone-like organoid produced according to a method described herein. In another embodiment there is provided a composition comprising an isolated population of iPSC-derived osteoblasts or bone-like organoids, wherein said cells express COL1A1 and COL1A2. In some embodiments, the isolated population of iPSC-derived osteoblasts or a bone-like organoids may also express one or more of MEPE, IBSP (bone sialoprotein 2), SPP1 (osteopontin) and DMP. Compositions of these embodiments may be suitable for bone repair or replacement. Accordingly, in another embodiment there is provided a composition according to the foregoing embodiments, for use in the repair or replacement of bone in a subject in need thereof. In another embodiment there is provided a method of repairing or replacing bone in a subject in need thereof comprising administering to the subject a therapeutically effective amount of a composition according to the foregoing embodiments. In another embodiment there is provided use of a composition according to the foregoing embodiments the manufacture of a medicament for repairing or replacing bone in a subject in need thereof.

A further embodiment relates to a composition comprising isolated population of chondrocytes or chondrocyte-like cells, or cartilage-like tissue or cartilage, or a combination thereof, and/or an isolated population of osteoblasts or a bone-like organoid produced according to a method described herein and a carrier such as polymer, hydrogel, bone scaffolding, bone substitute scaffolding. Other carriers include for example carrier comprises one or more of a group consisting of sodium hyaluronate, hyaluronic acid and its derivatives, gelatin, collagen, chitosan, alginate, buffered PBS, Dextran and polymers. For example, the carrier can be a carrier that is suitable for use in transplantation applications e.g. pharmaceutical grade carriers. The carrier can also be suitable for stabilizing the cells for transport and/or storage. Cells can for example be cryofrozen and/or tissues can be shipped at room temperature and/or any between room temperature and about 4° C. In an embodiment, the carrier is pharmaceutical grade.

In an embodiment, the isolated population is comprised in a composition comprising a diluent or carrier, optionally a pharmaceutical diluent. In an embodiment, the diluent is culture media, optionally comprising a cryopreservation agent such as glycerol and/or DMSO, serum and albumin, such as human serum albumin.

According to another embodiment, the three-dimensional aggregation of chondrocytes, or chondrocyte-like cells, cartilage-like tissue or cartilage or combination thereof, or the osteoblasts or a bone-like organoid produced according to the methods described herein may be decellularized in order to produce a decellularized scaffold.

In one embodiment, the composition can for example be in a slurry comprising dissociated cells for example for administration to a subject. In an embodiment, the composition may comprise other cells for example endothelial cells and/or fibroblasts for example for growth plate cell/cartilage transplantation.

A further aspect includes a cartilage or bone tissue product comprising cells and/or tissue described herein and a scaffold or membrane. For example, during transplantation applications, chondrocytes can be administered to a damaged area in combination with a membrane (e.g. tibial periosteum or biomembrane) or pre-seeded in a scaffold matrix. In an embodiment, the scaffold is a bone substitute. In another embodiment, the different types of chondrocytes, or chondrocyte-like cells, cartilage-like tissue or cartilage or combination thereof may be combined. For example, articular chondrocytes generated according to the methods of the invention may be arranged as a laminate with proliferative/resting zone chondrocytes which can further mature, hypertrophy and develop into bone.

Accordingly, cells and tissues, compositions and scaffolds generated according to the methods disclosed herein can for example be used for treating a subject afflicted with a joint or bone injury or disorder or alleviating or ameliorating symptoms associated with said joint or bone injury. In one embodiment there is provided a method of treating a chondral injury or defect or an osteochondral injury or defect in a subject in need thereof comprising a) deriving a composition comprising: chondrocytes, chondrocyte-like cells, cartilage-like tissue, or a combination thereof, or osteoblasts or bone-like organoid according to a method described herein, or a combination of said chondrocytes, chondrocyte-like cells, cartilage-like tissue, or a combination thereof and said osteoblasts or bone-like organoid, and b) administering the composition to the subject.

According to another embodiment, there is provided a composition comprising: chondrocytes, chondrocyte-like cells, cartilage-like tissue, or a combination thereof, or osteoblasts or bone-like organoid produced according to a method described herein, or a combination of said chondrocytes, chondrocyte-like cells, cartilage-like tissue, or a combination thereof and said osteoblasts or bone-like organoid, for use in treating a chondral injury or defect or an osteochondral injury or defect in a subject in need thereof.

According to another embodiment, there is provided use of a composition comprising: chondrocytes, chondrocyte-like cells, cartilage-like tissue, or a combination thereof, or osteoblasts or bone-like organoid produced according to a method described herein, or a combination of said chondrocytes, chondrocyte-like cells, cartilage-like tissue, or a combination thereof and said osteoblasts or bone-like organoid, in the manufacture of a medicament for treating a chondral injury or defect or an osteochondral injury or defect in a subject in need thereof.

Another aspect accordingly includes a method for ameliorating symptoms and/or treating a subject in need thereof comprising administering the population of cells and/or tissues described herein and/or inserting/implanting a product comprising said cells.

Uses of the cells, tissues and products are also provided in another aspect. In an embodiment, the disclosure provides use of the population of cells and/or tissues or composition or product described herein for ameliorating symptoms and/or treating a subject in need thereof.

In an embodiment, the cells and tissues, compositions and scaffolds for a use or method described herein for example that are to be administered to the subject are derived from autologous cells.

### Screening Methods

Candidate substances may be screened for their ability to promote, inhibit, maintain or otherwise play a role in endochondral development or osteochondral diseases or disorders using the methods described herein.

In another embodiment there is provided a method of testing candidate chondrogenic or osteogenic modulating substances, the method comprising:
a) carrying out a method for generating chondrocytes, chondrocyte-like cells, cartilage-like tissue, cartilage, or a combination thereof, or osteoblasts or bone-like organoid, as described herein, wherein said test substance is included in any one, or more, of the culture steps of the method;
b) assessing the effect of the test substance on chondrocyte or osteoblast proliferation, maintenance and/or differentiation compared to a control population generated in the absence of test substance; and
c) identifying the test substance as a candidate chondrogenic or osteogenic modulating substance if the test substance increases or decreases proliferation, and/or affects chondrocyte or osteoblast maintenance or differentiation compared to the control.

Chondrocytes, chondrocyte-like cells, cartilage-like tissue, or a combination thereof, or osteoblasts or bone-like organoid produced according to a method described herein or the cells employed to derive such cells/tissues may also be engineered to stably express a reporter gene operably linked to a promoter of a gene typically expressed in a chondrocyte (e.g. articular chondrocyte-PRG4, or hypertrophic chondrocyte collagen X) to provide a model cell, that can for example be used for testing for candidate substances. A large number of reporter genes are known in the art including for example fluorescent proteins (such as GFP, RFP, dsRed etc, luciferase). Reporter gene assays are versatile and sensitive methods and can be used to assay numerous candidate substances in high-throughput drug-screening programs.

In one embodiment, the screening methods may be carried out using cells derived from a subject from a subject with a bone or cartilage disease or disorder. In another embodiment the screening methods may be carried out using gene-edited cells engineered to have one or more disease-associated mutations or engineered to correct one or more disease associated mutations. In one embodiment the bone or cartilage disorder is selected from one of the following cartilage and bone disorders groups: FGFR3 chondrodysplasia group, Type 2 collagen group, Type 11 collagen group, Sulphation disorders group, Perlecan group, Aggrecan group, Filamin group and related disorders, TRPV4 group, Ciliopathies with major skeletal involvement, Multiple epiphyseal dysplasia and pseudoachondroplasia group, Metaphyseal dysplasias, Spondylometaphyseal dysplasias (SMD), Spondylo-epi-(meta)-physeal dysplasias (SE (M) D), Severe spondylodysplastic dysplasias, Acromelic dysplasias, Acromesomelic dysplasias, Mesomelic and rhizo-mesomelic dysplasias, Campomelic dysplasia and related disorders, Slender bone dysplasia group, Chondrodysplasia punctata (CDP) group, Neonatal osteosclerotic dysplasias, Osteopetrosis and related disorders, Osteogenesis imperfecta and decreased bone density group, Abnormal mineralization group, Lysosomal Storage Diseases with Skeletal Involvement (Dysostosis Multiplex group), Disorganized development of skeletal components group, Cleidocranial dysplasia and related disorders, and Dysostoses with predominant craniofacial involvement. In one embodiment the disease is osteogenesis imperfecta. In another embodiment, the cartilage disorder is associated with a mutation in COL2A1.

### Kits

Also provided herein are kits comprising one or more of a cell or tissue generated according to a method described herein, a product or composition comprising a cell or tissue generated, optionally comprising a further therapeutic agent, or optionally wherein the cell comprises a reporter system or other modification, according to a method described herein, a combination of at least two selected from an agonist, inhibitor, media, apparatus or other component that can be used in a method described herein and instructions for use, for example instructions on how to generate the cells, perform an assay or administer the cell, tissue, composition, or product, and a vial or other container for housing one of these aforementioned cells, tissues, compositions, products, agonists, inhibitors, medias etc.

### EXAMPLES

Herein the inventors have optimized a sclerotome to chondrocyte differentiation protocol. From a chondroprogenitor stage to 3D cartilage organoids, “chondronoids”, can be guided towards articular chondrocytes in chondrogenic medium with TGFβ3 or directed along the growth plate chondrocyte pathway to become hypertrophic chondrocytes. Hypertrophy can be stimulated and enhanced with thyroxine (T3). We show that when transplanted into immunodeficient mice, the hypertrophic chondrocytes within T3 treated organoids, can recapitulate growth plate maturation and endochondral bone formation, including the transition to human osteoblasts in the forming bone. Furthermore, when the hypertrophic chondronoids are cultured in vitro with osteogenic medium, we were able to show transdifferentiation to osteoblasts and extensive mineralization. At sequential steps in this novel in vitro model of human endochondral bone formation we performed global transcriptomics, identifying the gene signatures of these developmental stages during cartilage maturation, hypertrophy and transdifferentiation to osteoblasts. We identified the transcription factor networks involved in this maturation and changes in the extracellular matrix (matrisome) during this developmental sequence. This in vitro endochondral model system will facilitate studies exploring normal growth plate cartilage development and mechanistic studies on inherited diseases of cartilage and bone development and homeostasis. In addition, the homogeneous articular cartilage chondronoids will be valuable for studies on osteoarthritis and regenerative medicine approaches to articular cartilage repair.

### Materials and Methods

### Human Induced Pluripotent Stem Cell Lines and Maintenance

We used five independent fully characterized and validated control human induced pluripotent stem cell (iPSC) lines-feeder-dependent fibroblast-derived RM3.5 (Kao et al., 2016), peripheral blood mononuclear cell derived MCRIi001-A (Vlahos et al., 2019), and a gene-edited subclone, MCRIi001-A, expressing a SOX9-tdTomato reporter (Nur Patria et al., 2020), and fibroblast-derived feeder-independent lines MCRIi018-B (Howden et al., 2019) and MCRIi019-A (Kung et al., 2020). Feeder-dependent cells were routinely expanded on mitotically inactivated mouse embryonic fibroblasts (MEFs) in KnockOut DMEM/F-12 with 20% KnockOut Serum Replacement, 2 mM GlutaMax, 1% Non-Essential Amino Acid Solution, 0.1 mM β-mercaptoethanol (all from Thermo Fisher Scientific) and 50 ng/ml FGF2 (PeproTech) at 37° C. with 5% CO₂. Media was changed daily and cells passaged (1:6 split) every ˜3 days with 0.5 mM EDTA in PBS. Feeder-free cells were routinely expanded on Matrigel (Corning) coated plates in Essential 8 (E8) medium (Thermo Fisher Scientific). Media was changed daily and cells passaged (1:4-1:6) every 3-4 days with 0.5 mM EDTA in PBS.

### Sclerotome Differentiation

Undifferentiated hiPSCs at 70-90% confluency were dissociated into fine clumps using 0.5 mM EDTA in PBS and typically passaged into 6 well plates (˜2×10⁵ cells per well for RM3.5, MCRIi0018-B, MCRIi019-A and ˜0.8×10⁵ cells per well for MCRIi-001-A and MCRIi001-A-2) either pre-seeded with MEFs or pre-coated with Matrigel, and cultured for 24-48 hours in the appropriate expansion media. Differentiation to sclerotome was as described (Loh et al., 2016) modified by substituting the CDM2 basal medium with APEL2 (StemCell Technologies). On day 0, medium was changed to anterior primitive streak inducing medium consisting of APEL2 containing 30 ng/ml Activin A (R&D Systems), 4 μM CHIR99021 (Tocris), 20 ng/ml FGF2 (PeproTech) and 100 nM PIK90 (Merck Millipore). After 24 hours this medium was replaced with APEL2 containing 3 μM CHIR99021, 20 ng/ml FGF2, 1 mM A8301 (Tocris) and 0.25 mM LDN193189 (Cayman Chemical) to induce paraxial mesoderm for 24 hours. Early somite development was then induced with APEL2 containing 1 mM A8301 and 0.25 mM LDN193189, 3 mM C59 (Tocris) and 5 mM PD0325901 (Selleck Chemicals). After 24 hours, sclerotome induction was initiated with APEL2 containing 1 mM C59 and 2 mM purmorphamine (Sigma-Aldrich). After the first day of sclerotome induction (day 4) cells were dissociated from monolayer culture using 0.025% Trypsin/EDTA, resuspended in sclerotome differentiation media, 300 μl aliquots containing 2×10⁵ cells were dispensed into 96-well low-attachment, round-bottom plates (Corning), and pelleted by centrifugation at 400 g for 3 minutes using a swing-out rotor. Pellets were incubated in sclerotome differentiation media for a further 48 hours to complete sclerotome differentiation in the 96-well static culture format. Importantly, cells at the sclerotome stage before pelleting could be frozen in medium containing 10% DMSO, stored under liquid nitrogen and retrieved for subsequent studies, allowing well-characterized sclerotome to be stockpiled for differentiation consistency and larger scale experiments.

### Chondrocyte Differentiation and Maturation

Sclerotome to chondrocyte differentiation was in APEL2 medium containing 5% Protein Free Hybridoma Medium (PFHM II; Thermo Fisher) and 20 ng/ml FGF2. In some experiments BMP4 (R & D Systems) was included at 20 ng/ml. Pellets were routinely transferred to 6 cm non-adherent dishes (Greiner) at the end of either day 6 or day 20; 15-20 pellets per dish in 5 ml of medium with orbital rotation at 60 rpm. The medium was changed every 2-3 days. From day 20 (after 2 weeks of FGF2 treatment), differentiation/maturation continued in APEL2/5% PFHM with medium replaced every 2-3 days for the duration of the chondrogenic differentiation experiments as indicated. In some experiments pellets were treated with FGF2 for 4 weeks post-sclerotome.

Triiodothyronine (T3) is crucial for chondrocyte hypertrophy (Aghajanian et al., 2017) thus in some experiments differentiated chondrocytes were treated with 10 nM T3 (Sigma-Aldrich) for 10-21 days to induce chondrocyte hypertrophy. From day 20 (after 2 weeks of FGF2 treatment), differentiation/maturation continued in APEL2/5% PFHM (without FGF2) with medium replaced every 2-3 days. T3 was added when the pellets had consistent and even cartilage histology, which ranged between 4 and 6 weeks post-sclerotome chondrocyte differentiation. In some experiments, pellets were briefly treated with 0.25% trypsin/EDTA for 4 min at 37° C. with agitation to remove the small amount of non-cartilage tissue that occasionally remained on the surface of the chondrocyte pellets. These were allowed to recover in chondrocyte medium (APEL2/5% PFHM) for 1 week before analysis and/or T3 treatment and continued maturation.

In some experiments, differentiated chondrocytes were treated with 10 ng/ml TGFβ3 (R&D Systems) for up to 34 days to generate articular cartilage. From day 6, differentiation/maturation continued in APEL2/5% PFHM (with FGF2) for 7 days. From day 13, 10 ng/ml TGFβ3 was added (i.e. FGF2 and TGFβ3) with medium replaced every 2-3 days. At day 20, the pellets were transferred to rotary culture and cultured with 10 ng/ml TGFβ3 (R&D Systems) (and without FGF2) for a further 4 weeks.

### Osteogenic Differentiation

Hypertrophic chondrocytes can transdifferentiate into osteoblasts and osteocytes in vivo (Yang et al., 2014; Zhou et al., 2014). To recapitulate this later stage of endochondral bone formation in vitro, hypertrophic chondrocyte pellets in orbital culture were rinsed in PBS then cultured in DMEM, high glucose, GlutaMAX Supplement, pyruvate (ThermoFisher), 20% FBS (ThermoFisher), 10 mM β-glycerophosphate, 50 μg/ml ascorbic acid 2-phosphate, 50 μg/ml sodium ascorbate, 100 nM dexamethasone (osteogenic differentiation media) for up to three weeks. Some cultures were supplemented with 10 μM CHIR99021 for the first 7 days. Media was replaced every 2-3 days.

### In Vivo Transplantation to Generate Human Ectopic Bone

Cartilage organoids were treated with T3 for 1 week from day 35 then implanted subcutaneously into NBSGW mice (McIntosh et al., 2015) The organoid grafts were harvested after 13 weeks. All animal procedures were approved by the Murdoch Children's Research Institute Animal Ethics Committee (Approval A863).

### Histological Analyses

Cultured and in vivo grafted pellet organoids were fixed overnight at 4° C. in Confix (Australian Biostain) neutral-buffered formalin, then decalcified washed in 70% ethanol, and paraffin-embedded. After fixing, some in vivo grafted pellets were washed in water for 10 minutes then decalcified in 14% EDTA, pH 7.4 at 4° C. for 5 days before embedding. Serial 5 μm sections cut from the pellet centers were mounted on Superfrost Plus slides (Menzel-Gläser) and heated at 55° C. overnight. Sections were treated with xylene to remove the paraffin wax and an ethanol series comprising 100%, 90%, 70%, 50% ethanol then water to rehydrate the sections. Sections were stained with toluidine blue to detect a cartilage proteoglycan matrix or safranin O/fast green for cartilage and bone following standard protocols. Osteogenic pellet sections were also stained with von Kossa to detect mineralization. Images were captured using a Leica DM 2000 LED microscope with Leica Application Suite (LAS) software version 4.9.0 or Zeiss Axio Imager Z2 with Zen 3.1 Blue Edition software.

### Immunostaining

Formalin fixed and paraffin-embedded sections were immunostained for collagen II (MAB8887; Sigma-Aldrich; 1:150 dilution), collagen I (LF68; Kerafast ENH018-FP; 1:100), collagen X (Cameron et al., 2015) (1:100), PRG4 (clone 9G3; Merck) (1:100) and Ku80 (Cell Signaling) (1:300). For collagen II staining, antigen retrieval used 2 mg/ml porcine pepsin (Sigma) freshly prepared in 1 M Tris/HCl, pH 2.0 for 30 minutes at 37° C., followed by digestion for 30 minutes at 37° C. with 0.2% hyaluronidase (Sigma-Aldrich) in PBS. Collagen X antigen retrieval involved heating to 60° C. in 10 mM Tris/1 mM EDTA/0.05% Tween-20, pH 9.0 (Tris/EDTA) for 30 min, cooling to room temperature for 30 minutes, followed by hyaluronidase digestion and collagen I and Ku80 staining followed antigen retrieval at 60° C. in Tris/EDTA as above. Sections to be stained with Ku80 were then permeabilized with 0.05% Triton X-100. Sections were blocked with 3% BSA in PBS for 1 hour at room temperature, then incubated with primary antibodies in 1% BSA in PBS for 16 hours at 4° C. Appropriate species specific Alexa Fluor 488 and 594 secondary antibodies (ThermoFisher) were diluted 1:200 in 1% BSA in PBS with 2.5 μg/ml DAPI, and sections incubated for 1 hour at room temperature. Slides were washed with PBS and coverslips mounted using Shandon Immumount (ThermoFisher). Images were captured as described above. For collagen X intracellular colocalization studies sections were treated with 0.2% hyaluronidase at 37° C. for 30 minutes then permeabilized with 0.05% Triton X-100. The collagen X antibody was used at 1:100, 58K (PA1-9000; ThermoFisher) at 1:50, GRP94 (PA5-18534; ThermoFisher) 1:200 and LAMP2 (ab25631; Abcam) 1:50. Images were captured on a Zeiss LSM900 Airyscan 2 confocal microscope with Zen 3.1 Blue Edition software.

### Transmission Electron Microscopy

Pellets were washed with PBS and fixed in 0.1 M sodium cacodylate containing 2.5% glutaraldehyde at 4° C. Samples were post-fixed in 1% aqueous osmium tetroxide, dehydrated in an alcohol series, and embedded in Epon 812. Seventy nanometer ultrathin sections were cut and observed on a Tecnai F30 with an extraction voltage of 200 kV. Micrographs were taken using a Gatan UltraScan 1000.

### Micro-CT

Pellets were formalin fixed, stored in 70% ethanol at 4° C., then scanned using micro-CT (uCT50, Scanco Medical AG) with an 0.5 mm A1 filter at an energy of 70 kVp, intensity of 200 μA, integration time of 300 ms, and a voxel (native) resolution of 3 μm. Bone morphometry parameters were calculated using a common threshold of 210/1000 for bone tissue within the volume of interest.

### Quantitative RT-PCR

RNA was extracted from monolayer cultures using TRIzol (Qiagen) following the manufacturer's instructions. Typically 100-200 ng total RNA was used for cDNA synthesis in a total volume of 20 μl using the QuantiTect® Reverse Transcription kit (Qiagen) according to the manufacturer's instructions. Quantitative RT-PCR (qPCR) was performed in triplicate with the Brilliant III Ultra-Fast SYBR Green QRT-PCR Kit (Agilent) using 10 μl reactions consisting of 1x Brilliant III Ultra-Fast SYBR Green QRT-PCR Master Mix, 1 μl cDNA, and 1 μM each primer. Amplification was performed in a 384 well plate format on the LightCycler 480 Instrument II (Roche Life Sciences), using a thermocycling protocol involving 40 cycles of denaturation at 95° C. for 20 seconds, annealing at 55° C. for 20 seconds, and amplification at 72° C. for 20 seconds. Gene-specific primers were designed using Primer-BLAST (NCBI). To avoid amplification from genomic DNA, primer sets were designed to span multiple exons and PCR products analyzed by agarose gel electrophoresis to confirm single products corresponding to the predicted cDNA amplicons for each gene. Analyses used LightCycler 480 Software (release 1.5.1.62). Human beta-actin (ACTB) was routinely used as a house-keeping reference gene. Graphs were drawn using GraphPad Prism 9.

### Bulk Population RNAseq

For RNAseq, organoids (2-3 pooled) were snap frozen in liquid nitrogen, pulverized using a liquid nitrogen-cooled tissue grinder and RNA extracted with TRIzol, followed by purification using Direct-zol RNA Microprep kit spin columns (Zymo Research) according to the manufacturer's instructions. RNA samples were quality controlled and sequenced at the Translational Genomics Unit, Murdoch Children's Research Institute. Libraries were constructed using Illumina Stranded mRNA Prep kits and sequenced using a NextSeq 500 to obtain ˜20×10⁶ 75 bp paired-end reads per sample or a NovaSeq 6000 for 150 bp paired-end reads. Reads were aligned to hg38 using a Bpipe (Sadedin et al., 2012) RNAseq pipline that incorporated FastQC quality control, adaptor trimming with Trimmomatic v.0.35 (Bolger et al., 2014), mapping with STAR 2.7.3a (Dobin et al., 2013), summarizing reads over genes with featureCounts (Liao et al., 2014), and MultiQC (Ewels et al., 2016) to summarize the analyses. Downstream analyses and identification of differentially expressed genes used the EdgeR Bioconductor package (Law et al., 2016). Genes with expression levels of at least one count per million in at least three samples were kept for further analysis. The data were TMM normalized and voom transformed. Differential expression was identified with robust paired moderated t tests using limma (Law et al., 2016). Graphical visualisations used the gplots, tidyverse, ggplot2, and EnhancedVolcano packages. Gene set enrichment analyses used the Broad Institute's MSigDB gene sets (https://www.gsea-migdb.org/gsea/msigdb/) and the clusterProfiler (Yu et al., 2012) and enrichplot packages. Protein interactomes were visualized using the online STRING tool (https://string-db.org/).

### Example 1. Generation of Sclerotome

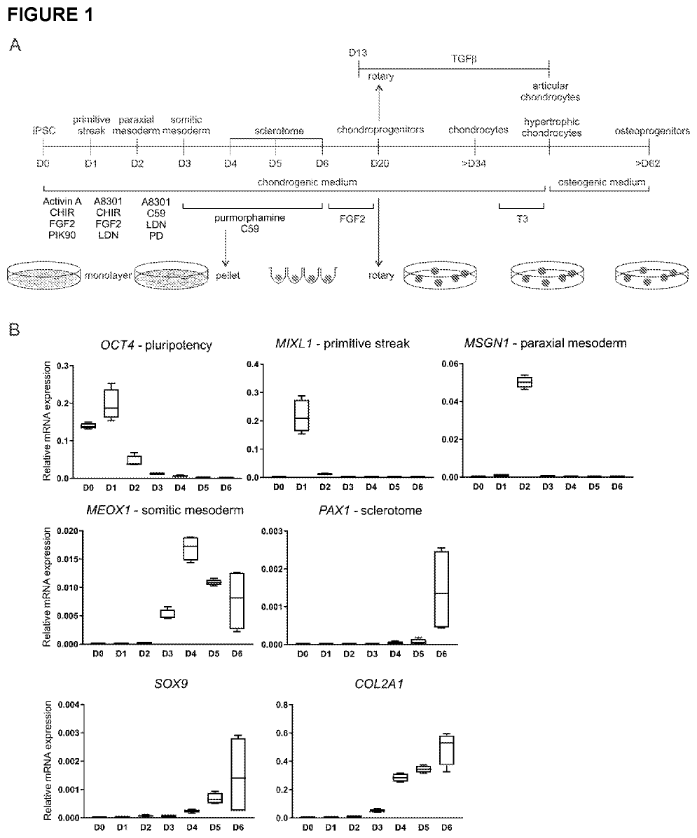

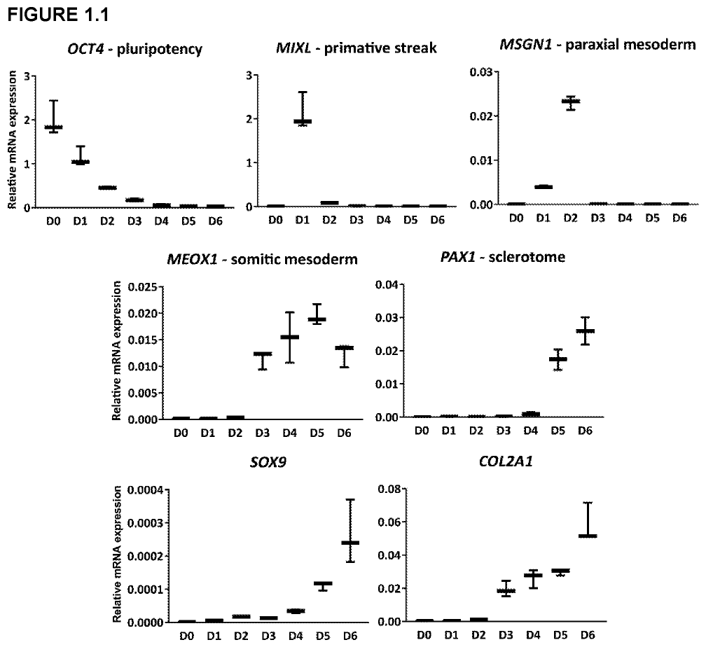

Our goal was to develop and optimize an efficient and reproducible method to differentiate human iPSCs to chondrocytes that could either mature to hypertrophy then transdifferentiate to osteoprogenitors, or be directed towards an articular cartilage phenotype. The first six days of our protocol (FIG. 1A) are based on a published method to generate sclerotome (Loh et al., 2016). We found this method to be robust and reproducible. We monitored expression of stage specific markers in two iPSC lines using qRT-PCR and saw the pluripotency marker OCT4 gradually downregulated, and the primitive streak, paraxial mesoderm, somitic mesoderm and sclerotome makers, MIXL1, MSGN1, MEOX1 and PAX1 respectively, up- and down-regulated at the appropriate developmental stages (FIG. 1B, FIG. 1.1). The key chondrocyte transcription factor SOX9 and cartilage collagen COL2A1 were upregulated in somitic mesoderm and sclerotome (FIG. 1B, FIG. 1.1).

### Example 2. FGF2 Promotes Sclerotome to Chondrocyte Differentiation

It is widely recognized that high-density pellet culture favors maintaining the chondrocyte phenotype ex-vivo and so we transferred cells into pellet culture, 2×10⁵ cells/pellet, at the end of day 4. In our first experiments we supplemented the pellet cultures for two weeks post-sclerotome with 20 ng/ml BMP4 to promote chondrocyte differentiation (Adkar et al., 2019; Loh et al., 2016) but found that key cartilage genes were not highly expressed (data not shown).

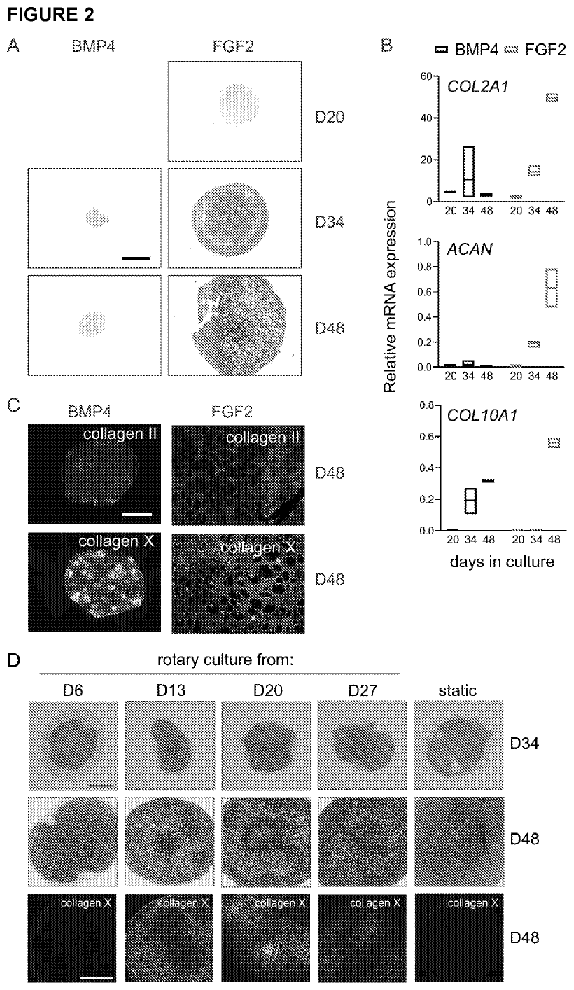

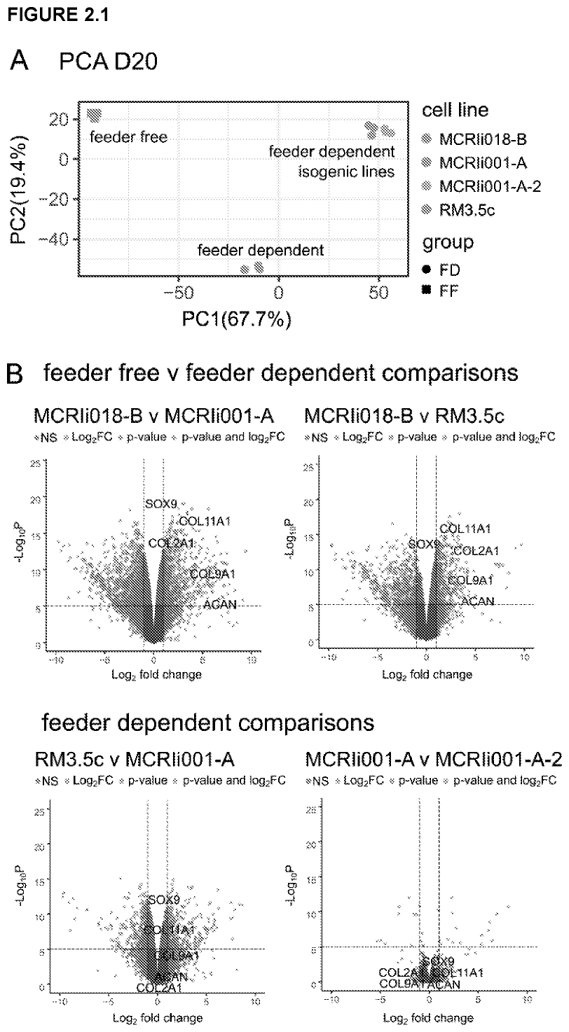

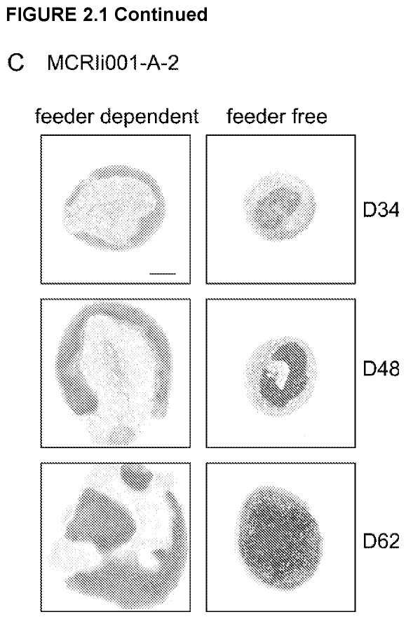

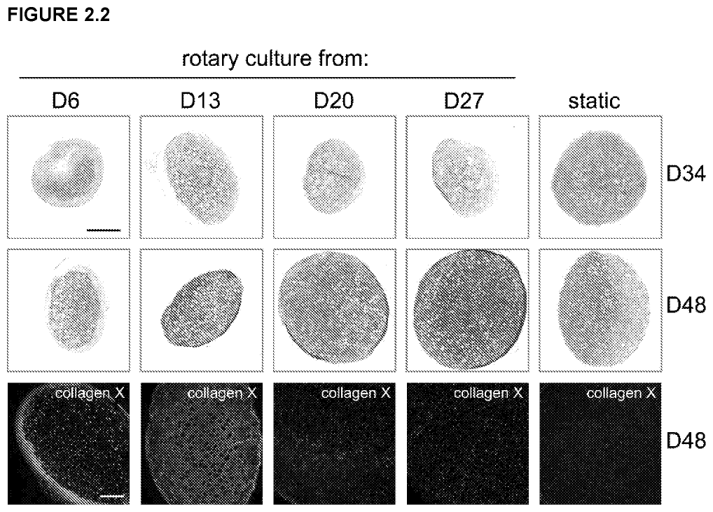

We then compared supplementing post-sclerotome with 20 ng/ml BMP4 or 20 ng/ml FGF2 for 14 days followed by extended culture without additional growth factors. We found that changing the culture medium on cell pellets in 96 well plates was time consuming and difficult to automate without damaging the pellets so we also planned to compare chondrocyte differentiation in static pellets and pellets transferred to rotary culture at the end of day 6; however, BMP4 treated pellets were fragile and could not be maintained in rotary culture. In static culture FGF2 treated pellets showed strong toluidine blue staining throughout at day 34 reflecting accumulation of extracellular matrix proteoglycans typical in cartilage, while BMP4 treated pellets were much smaller and only isolated areas were toluidine blue positive (FIG. 2A). FGF2 treated pellets in rotary culture also developed into organoids that stained throughout with toluidine blue but differentiation was delayed when compared to static FGF2 treated pellets (FIG. 2A). In FGF2 treated cultures COL2A1 and ACAN gene expression was consistently higher than in BMP4 cultures, but the hypertrophic cartilage gene COL10A1 was expressed earlier in BMP4 treated cultures (FIG. 2B). By day 48 FGF2 treated pellets, both static and rotary, had deposited an extensive and uniform collagen II extracellular matrix while BMP4 treated pellets had only small foci that were positive for collagen II (FIG. 2C). Similarly, collagen X extracellular staining was strong in FGF2 treated static pellets, particularly in areas where the cells were larger and appeared hypertrophic; in BMP4 treated pellets collagen X was only seen in small, localized regions (FIG. 2C).

Based on these data subsequent differentiation experiments included establishing pellet culture at the end of day 4 and supplementing with 20 ng/ml FGF2 for 14-28 days post sclerotome differentiation.

### Example 3. Cell Line and Culture Format Influence Post-Sclerotome Chondrocyte Differentiation

To assess the variability in chondrocyte differentiation between iPSC lines we differentiated four iPSC lines in parallel to sclerotome then treated for FGF2 for 14 days and harvested pellets for RNAseq. One iPSC line, MCRIi018-B, was grown in feeder free conditions, while three lines, MCRIi001-A, MCRIi001-A-2 and RM3.5c, were feeder dependent lines. MCRIi001-A and MCRIi001-A-2 are isogenic lines with MCRIi001-A-2 modified to express a heterozygous SOX9-TdTomato fluorescent reporter (Nur Patria et al., 2020). Principal component analysis showed the samples formed three distinct clusters according to the cell line with the two isogenic lines clustered closely together (FIG. 2.1A). We compared gene expression in feeder-dependent lines with the feeder-free line and found >1800 genes were differentially expressed between the feeder-free line, MCRIi018-B, and feeder-dependent MCRIi001-A and >1000 between MCRIi018-B and RM3.5c (log FC >1, adj.P.value <0.00001, FIG. 2.1B). Fewer genes were differentially expressed in the feeder-dependent comparison (˜750) and only 26 between the isogenic feeder-dependent lines (FIG. 2.1B). Key chondrocyte genes, SOX9, COL2A1, COL9A1, COL11A1 and ACAN were more highly expressed in the feeder-free line than in any of the feeder-dependent lines (FIG. 2.1B) suggesting the possibility that iPSC lines maintained in feeder-free conditions could differentiate more efficiently.

To test this, feeder-dependent MCRIi001-A-2 was adapted to grow in feeder-free conditions and then the feeder-free and feeder-dependent versions were differentiated in parallel. Both feeder-dependent and feeder-free lines were able to differentiate into cartilage but at day 62, pellets from feeder-dependent MCRIi001-A-2 iPSCs still contained a large amount of non-cartilage tissue (toluidine blue negative) while pellets from feeder-free MCRIi001-A-2 were almost entirely cartilage with just a thin layer of non-cartilage cells around the outside (FIG. 2.1C). Subsequent differentiation experiments all used iPSC lines that were adapted to feeder-free conditions.

Our earlier comparison of chondrocyte differentiation in pellets cultured in static conditions versus rotary culture from day 6 showed that rotary culture slowed chondrocyte development (FIG. 2). We differentiated iPSC line MCRIi001-A-2 to sclerotome, forming pellets at day 4. Some pellets were transferred to rotary culture at day 6, day 13, day 20 and day 27, and some pellets were maintained in static culture. At day 34 pellets in rotary culture from day 6 had a cartilage center surrounded by a thick layer of non-cartilage tissue while pellets from all the other treatments had just a thin layer of non-cartilage cells on the outside (FIG. 2.2). At day 48 all the pellets had matured further but pellets in rotary culture from day 6 retained a surface layer of non-cartilage cells. Pellets that were in rotary culture from day 13, day 20 and day 27 had noticeably enlarged chondrocytes and collagen X staining confirmed that these were collagen X expressing hypertrophic chondrocytes (FIG. 2.2A). We confirmed this finding in a second iPSC line, MCRIi019-A, but in this line the highest collagen X expression at day 48 was in pellets transferred to rotary culture on day 13 (FIG. 2. 2A). Taken together these experiments confirm that the novel differentiation protocol described herein works in multiple iPSC lines, individual cell lines mature to chondrocytes and hypertrophy at different rates and transfer to rotary culture influences chondrocyte maturity.

### Example 4. Triiodothyronine Enhances Spontaneous Chondrocyte Hypertrophy

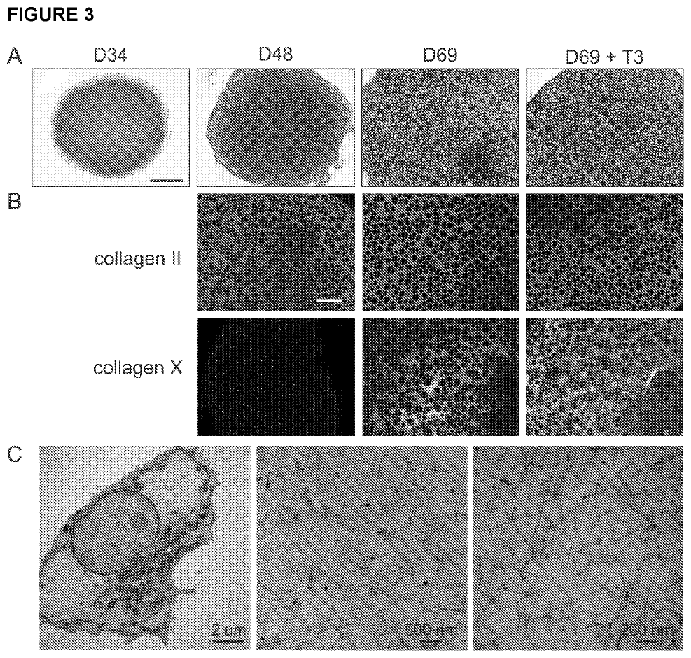

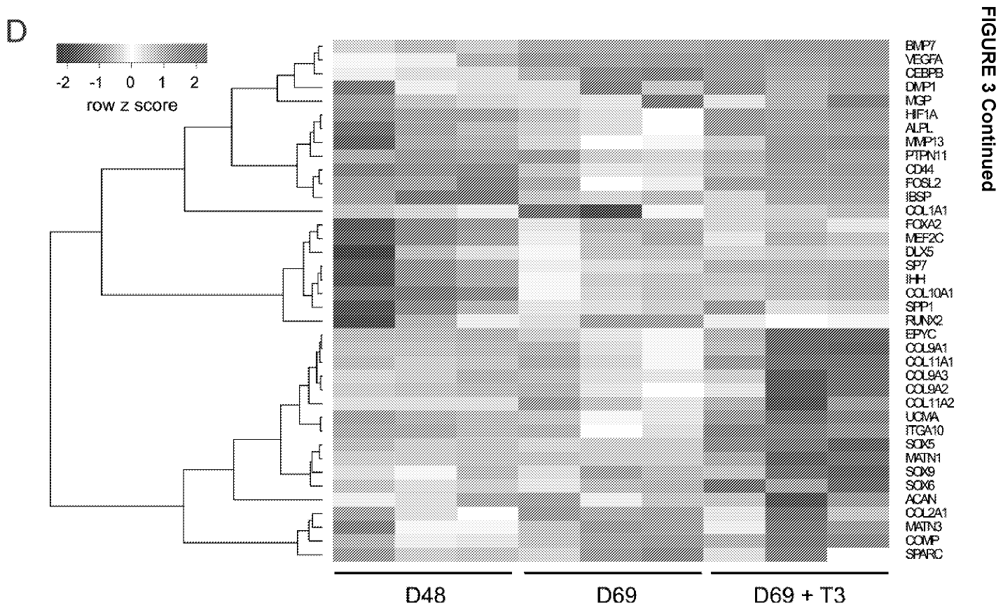

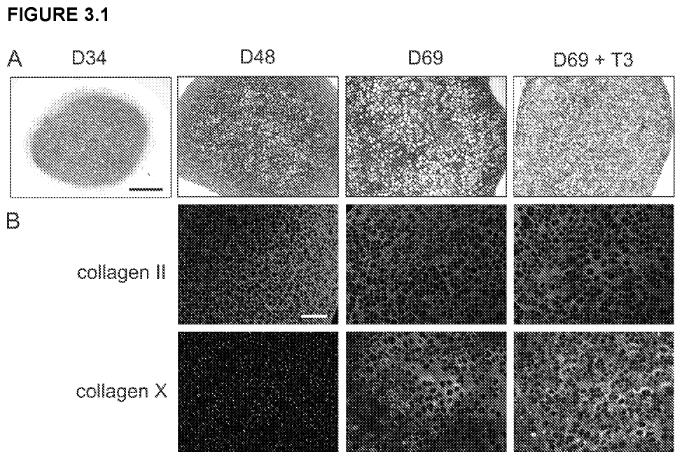

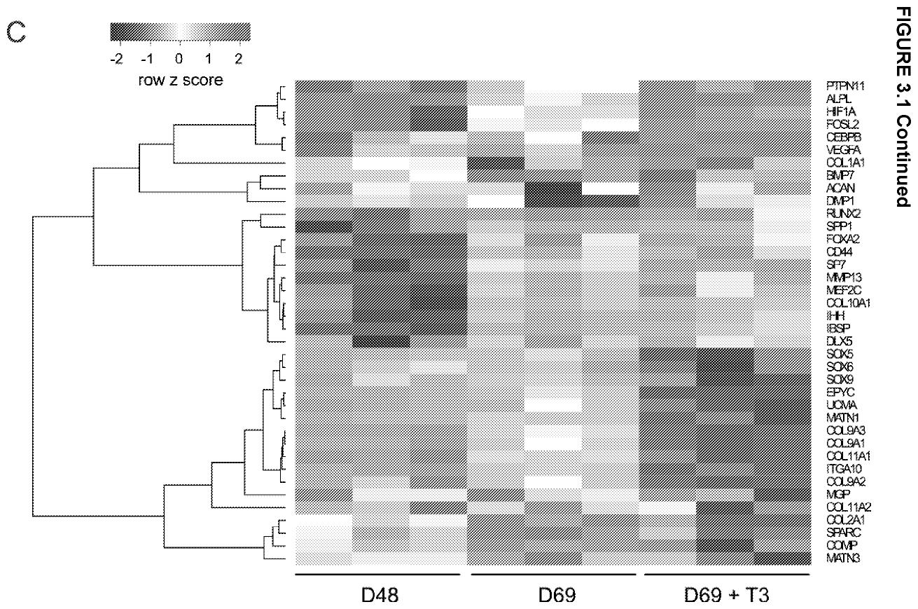

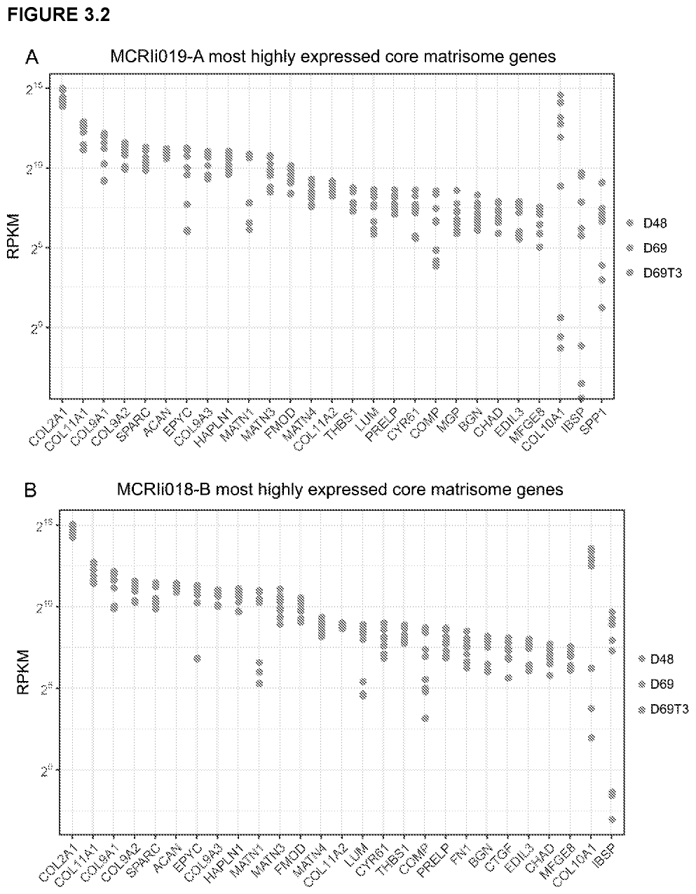

To understand more about the changes that occur as iPSC-derived chondrocytes mature towards hypertrophy we monitored differentiation in two iPSC lines, MCRIi019-A and MCRIi018-B. In these experiments sclerotome pellets were transferred to rotary culture at day 6 then supplemented with FGF2 for 28 days. Chondronoids matured for a further 35 days in chondrogenic medium. Although we had seen spontaneous hypertrophy and collagen X expression in previous experiments (FIG. 2), triiodothyronine (T3) is crucial for chondrocyte hypertrophy and transdifferentiation into osteoblasts in the mouse secondary ossification center (Aghajanian et al., 2017) and so we treated some pellets with T3 from day 48 to day 69. By day 48 the pellets were pure cartilage and had deposited an extensive proteoglycan and collagen II rich ECM (FIG. 3A, B, C, FIG. 3.1A, B). The chondrocytes had become larger and collagen X was abundant in the ECM by day 69 clearly demonstrating spontaneous maturation to hypertrophy (FIG. 3B, FIG. 3.1B).

Cartilage expresses a distinctive repertoire of extracellular matrix proteins that endow the tissue with its unique properties and reflect chondrocyte subpopulations. To gain a comprehensive and unbiased assessment of ECM gene expression during iPSC-derived chondrocyte maturation in vitro, we interrogated our RNAseq transcriptome data at day 48, 69 and day 69+T3 against the core matrisome gene set (Nabi et al., 2016). At all developmental stages from day 48 to hypertrophy in both cell lines members of the cartilage collagen family which co-assemble to form collagen fibril supramolecular assemblies (COL2A1, COL11A1, COL9A1, COL9A2 and COL9A3) are among the ten most highly expressed components (FIG. 3.2). Likewise, the other key cartilage ECM molecules, aggrecan (ACAN) and HAPLN1 which links the aggrecan to the hyaluronan polymers, and cartilage matrilines (MATN1, MATN3) are strongly expressed throughout this developmental sequence. Concomitant with hypertrophy (day 69 and day 69+T3) expression of the core matrisome hypertrophy markers, COL10A1 and IBSP, are upregulated. Highly expressed sentinel cartilage genes downregulated by T3 include COL11A1, COL9A1, COL9A2, COL9A3, EPYC, MATN1, LUM, COMP and EDIL3 (FIG. 3.2) consistent with preparing to transdifferentiate to osteoblasts.

While cartilage maturation to hypertrophy between day 48 and day 69 was clear from the histology and immunostaining, the chondronoids appeared similar with and without T3 at day 69 (FIG. 3B, FIG. 3.1B). Despite this similar appearance, thousands of genes were differentially expressed between treatments at day 69 in our RNAseq data. We looked at expression of selected cartilage, hypertrophic cartilage and bone markers and saw that hypertrophic cartilage markers such as COL10A1, IHH, MEF2C, SP7 and SPP1, were upregulated between day 48 and day 69 (FIG. 3D, FIG. 3.1C). T3 treatment enhanced expression of late hypertrophic cartilage markers among which were VEGFA, DMP1, HIF1A, MMP13, and ALPL, and downregulated canonical cartilage genes such as COL2A1, COL9A1, COL11A1, SOX9, SOX6, and SOX5 (FIG. 3D, FIG. 3.1C). We concluded that T3 enhanced the spontaneous maturation to hypertrophic chondrocytes in our chondronoid cultures.

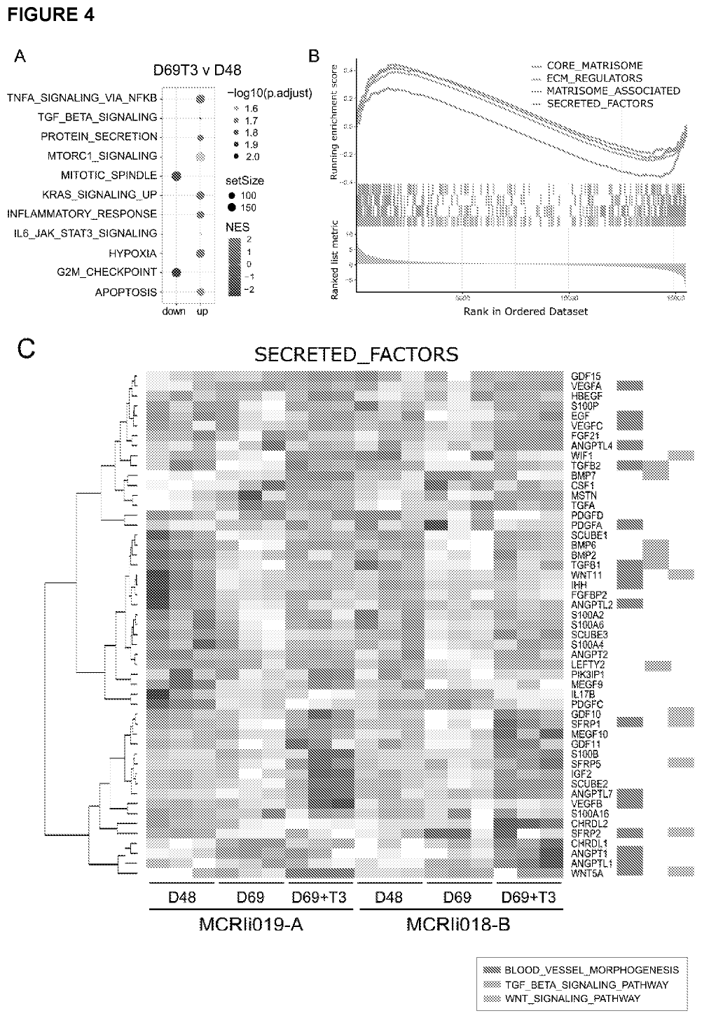

We explored the global transcriptome changes during chondrocyte transition to hypertrophy using Gene Set Enrichment Analysis (GSEA). Significantly enriched Hallmark gene sets (MSigDB) relevant to cartilage maturation indicated reduced cell division in hypertrophic chondrocytes and increased hypoxia and apoptosis (FIG. 4A). Other enriched gene sets important in cartilage development and arthritis but less thoroughly studied in growth plate maturation include TNFA, MTORCI, KRAS and JAK/STAT3 signaling (FIG. 4A).

Enriched C2 (curated) gene sets included the core matrisome genes and three other matrisome related gene sets (FIG. 4B). These matrisome gene sets have members that are highly upregulated and members that are highly downregulated reflecting dynamic changes in the ECM during chondrocyte hypertrophy. We focused on the secreted factors because they may offer additional insights into the regulatory steps involved in hypertrophic differentiation and initiation of chondrocyte trans-differentiation to osteoblasts. Upregulation of the TGFβ pathway, shown by GSEA analysis of the complete transcriptome dataset (FIG. 4A) is confirmed by upregulated KEGG TGFβ pathway components, TGFβ1, TGFβ2, BMP2, BMP6, BMP7, and LEFTY2 in the matrisome secreted factors (FIG. 4C), and is consistent with studies showing that TGFβ signaling positively regulates hypertrophy and angiogenesis in growth plate cartilage (Sue Yoshi et al., 2012). Angiogenesis and WNT signaling pathway members in the secreted factors gene set are dynamically and differentially expressed during maturation to hypertrophy in our dataset. Upregulated angiogenic factors (GOBP blood vessel morphogenesis gene set) include VEGFA, VEGFC, PDGFA, ANGPTL2 and ANGPT2 while SFRP1, WNT5A, ANGPT1 and VEGFB are downregulated (FIG. 4C). The angiogenic pathway has partial overlap with the KEGG WNT signaling pathway, where WNT11 and WIFI are upregulated and GDF10, SFRP1, SFRP5, SFRP2, and WNT5A are downregulated with hypertrophy (FIG. 4C).

The most highly expressed secreted factor gene upregulated during hypertrophy is FGFBP2. A role for this FGF binding protein has not been reported in cartilage and bone development and our data suggests it could regulate the biological activity of the FGF family during endochondral development. Other dynamically regulated secreted factors in the in vitro cartilage maturation model are members of the S100 calcium binding protein family including S100A2, S100A6, S100A4, S100P, S100B, and S100A16 (FIG. 4C). S100 proteins have many roles in regulating cell and tissue functions and in cartilage are implicated in TGFβ, and PI3/AKT pathways, Ca²⁺ homeostasis and mechanoenzyme through TRPV4 (Diaz-Romero and Mesic, 2017). SCUBE proteins modulate growth factor signaling and all three are differentially expressed during maturation to hypertrophy (FIG. 4C). In zebrafish Scube1 and Scube2 promote Vega signaling (Tsao et al., 2021) and loss of function SCUBE3 mutations and the resultant impaired BMP signaling underlie a human skeletal disorder (Lin et al., 2021).

### Example 5. Transcription Factor Expression During iPSC Chondrogenic Maturation

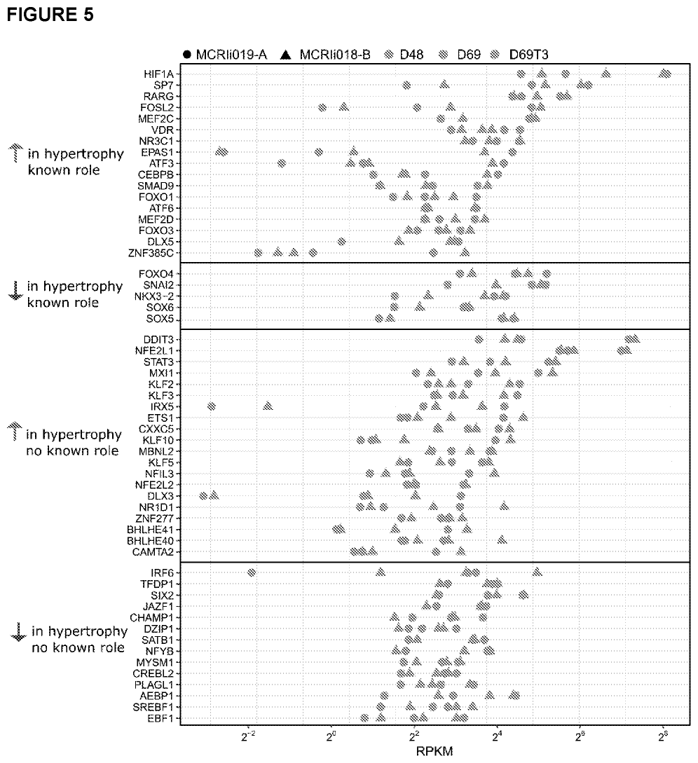

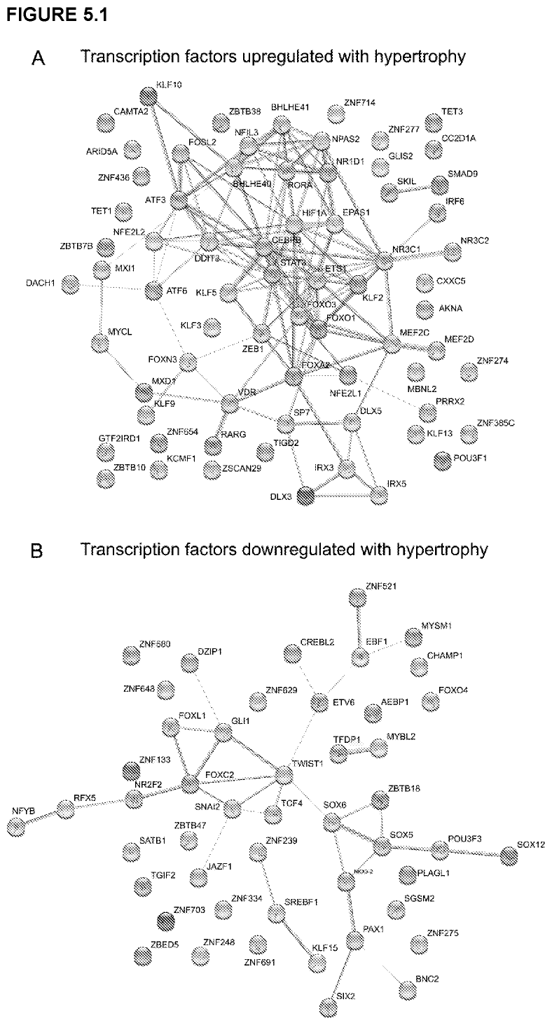

Normal skeletal development is controlled by complex temporal and spatial transcription factor circuitry that determines cell fate decisions (Hate et al., 2017; Arseniy et al., 2009; Kroonenberg, 2003; Liu et al., 2017; Tsang et al., 2014). Analyzing transcription factor (TF) regulation during iPSC differentiation to mature hypertrophic chondrocytes will allow us to determine how closely the in vitro pathway recapitulates in vivo growth plate cartilage maturation and so we interrogated our RNAseq data at sequential stages of chondrocyte maturation (day 48, day 69 and day 69+T3) using a human TF catalogue (Lambert et al., 2018). To reduce the complexity of this analysis we filtered the data to include TFs that were up- or downregulated at least 2 fold (Adj.P.value <0.05) and expressed at average RPKM >4 in at least one experimental group (cell line, day/treatment). Using these criteria 118 TF genes were differentially expressed during chondrocyte maturation (FIG. 5, FIG. 5.1). The expression patterns of TFs with established roles in chondrocyte differentiation and maturation demonstrated that the in vitro process closely recapitulated their developmental regulation. Highly expressed TFs such as the master transcriptional mediator of the adaptive response to hypoxia, HIF1A (HIF1α), and EPAS1 (HIF2α), a regulator of VEGF expression, are upregulated with hypertrophy (FIG. 5). The transition to hypertrophy is marked by upregulation of SP7 (osteria), RARG (retinoic acid receptor) (Shimon et al., 2019) and MEF2C which is important for endochondral differentiation of mesenchymal progenitor cells (Dreher et al., 2020). Likewise, hypertrophic upregulation of CEBPB (C/EBPB) is consistent with its suggested role in suppressing early chondrocyte differentiation and stimulating hypertrophic markers (Okuma et al., 2015). The coordinated expression of these (and other) TFs and their potential functional interactions in the regulatory circuitry of our iPSC chondrocyte differentiation and maturation pathways is emphasized by the known and predicted protein interactions shown in the STRING analysis (FIG. 5.1A). Of the 17 upregulated and most highly expressed TFs with established roles in chondrocyte maturation (FIG. 5), 15 are interactors. Many have multiple predicted interaction partners and HIF1A, CEBPB, EPAS1, FOXO1, FOSL2, MEF2C, ATF3 and SP7 sit at apparent major regulatory nodes (FIG. 5.1) (Liu et al., 2017).

SOX5 and SOX6 which are part of the SOX9/SOX5/SOX6 master chondrogenic trio (Liu et al., 2017) are coordinately downregulated (FIG. 5) along with SOX9 (Log FC=−0.83, adj.P.value=8.89×10⁻⁷) consistent with the transition to hypertrophy. GLII (FIG. 5.1) and GLI3 (Log FC=−0.72, adj.P.value=3.95×10⁵) are also downregulated with hypertrophy. GLI factors act in concert with SOX9 to suppress Col1a1 expression in proliferating mouse chondrocytes (Leung et al., 2011), while in hypertrophic chondrocytes elevated FOXA2 competes with SOX9 binding to the Col10a1 promoter and activates collagen X expression (Tan et al., 2018). FOXA2 is upregulated in our iPSC-derived hypertrophic chondrocytes (FIG. 5.1) suggesting a similar co-regulation network between SOX9-GLI cooperation in proliferating chondrocytes and SOX9-FOXA competition in hypertrophic chondrocytes is also relevant in human chondrocyte differentiation.

We also identified many TFs less well recognized in cartilage development that were strongly up- or downregulated with hypertrophy (FIG. 5, FIG. 5.1). Upregulated TFs associated with regulatory interaction nodes included ETS1, STAT3, DDIT3 and members of the KLF family (KLF2, KLF5, and KLF10). Also upregulated during maturation were several TFs associated with the circadian rhythm pathway, NFIL3, NR1D1, BHLHE41, and NPAS2, along with NR3C1 (glucocorticoid receptor) and FOXO1 (FIG. 5, FIG. 5.1). Our data also identify other upregulated transcription factors with currently poorly described roles in chondrogenesis and chondrocyte maturation that are potentially important including IRX3, IRX5, DLX3 and CXXC5.

### Example 6. TGFβ3 Drives iPSC-Derived Pre-Chondrocytes to an Articular Chondrocyte Fate

Having shown that our iPSC differentiation protocol produced growth plate chondrocytes that could transition to hypertrophic chondrocytes, we next tested if manipulating the protocol would allow us to produce chondrocytes from the articular cartilage lineage. Cell lineage-tracing experiments show that articular and growth plate chondrocytes are derived from common mesenchymal precursors and a specific fate decision to form articular chondrocytes occurs in early chondrocytes (Decker et al., 2015, 2014; Soda et al., 2010; Zhang et al., 2011). Members of the TGFβ superfamily have important roles at many chondrogenesis stages (Cleary et al., 2015; Pogue and Lyons, 2006; Wan and Cao, 2005; Wang et al., 2014) and TGFβ3 has been used in several iPSC chondrogenesis protocols (Dakar et al., 2019; Craft et al., 2015; Nakajima et al., 2018; O'Connor et al., 2020; Wu et al., 2021). A recent study demonstrated that mouse iPSC-derived chondroprogenitors treated with TGFβ3 for four weeks expressed the sentinel articular chondrocyte marker PRG4 (O'Connor et al., 2020). Based on these data suggesting an early endochondral/articular cell fate decision and a role for TGFβ3 in articular chondrocyte specification in vitro we treated iPSC-derived chondroprogenitors with TGFβ3 for five weeks starting at day 13.

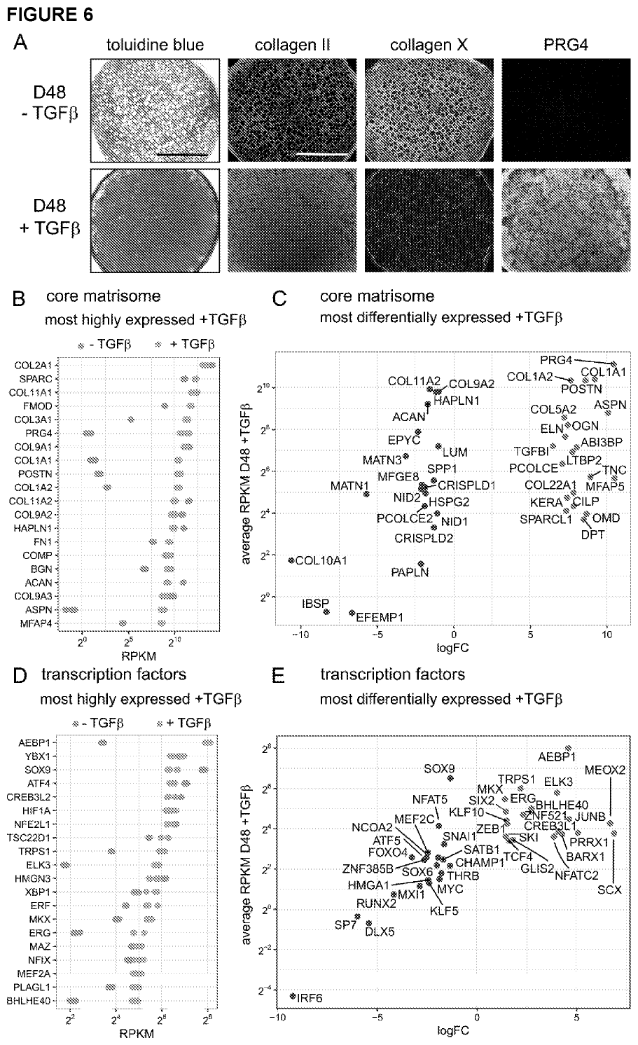

At day 48 we saw profound differences in cell morphology and gene expression between TGFβ3 treated and untreated chondronoids. Untreated D48 organoids contained large chondrocytes strongly expressing collagen II and the hypertrophic marker collagen X but not expressing PRG4, an articular chondrocyte marker (FIG. 6A). By contrast, chondroprogenitors treated with TGFβ3 developed into a distinct population of smaller chondrocytes, also expressing collagen II, but no extracellular collagen X (FIG. 6A) indicating they had not developed down the endochondral chondrocyte pathway. High uniform PRG4 immunostaining suggested that these TGFβ3-treated cells were articular chondrocytes (FIG. 6A).

The primary roles of permanent articular cartilage are weight bearing and facilitating joint articulation, whereas the transient growth plate cartilage serves primarily to drive pre-pubertal longitudinal bone growth through endochondral ossification. These different functions are reflected in the composition of their extracellular matrices. Thus, we compared mRNA expression at day 48 in chondronoids either treated with TGFβ3 to promote articular cartilage development or left untreated to mature towards hypertrophy. Canonical cartilage components COL2A1, COMP, COL9A1, COL9A2, COL9A3, and COL11A1 are highly expressed in both TGFβ3 treated and untreated chondronoids and either not differentially expressed (COMP) or downregulated 2 fold or less (FIG. 6B, C). By contrast, other core matrisome genes highly expressed in TGFβ3 treated chondronoids are strongly upregulated compared to untreated chondronoids. These include the sentinel articular cartilage marker, PRG4, articular cartilage proteins ASPN, CILP and CILP2 a protein associated with elastic fibers (FIG. 6B, C; Supplementary table 1). Other elastic fiber components, ELN, LTBP2, TGFBI and MFAP5, are among the 20 most highly upregulated genes (FIG. 6C), consistent with an articular cartilage phenotype (Botanic et al., 2019). EMILIN1, EMILIN3, FBLN1 and FBLN3, also elastic fiber components, are strongly upregulated (Supplementary table 1). Importantly, ECM proteins related to cartilage hypertrophy and endochondral ossification, such as COL10A1, IBSP, SPP1, and MATN3, are among the most downregulated in chondronoids differentiated down the articular cartilage pathway with TGFβ3 (FIG. 6C). Other changes consistent with an articular cartilage ECM included down-regulation of ACAN and the associated link protein HAPLN (FIG. 6C).

Compared to what is known about the transcription factors (TFs) that are dynamically expressed in maturing growth plate, relatively little is known about the TFs crucial for developing articular chondrocytes. Analysis of core matrisome gene expression in TGFβ3 treated chondronoids was consistent with known changes between articular and growth plate expression suggesting that our data set could be mined to identify key articular cartilage TFs. ERG, a TF enriched in articular cartilage (Iwamoto et al., 2007) and required for articular cartilage integrity (Otha et al., 2015), is highly expressed and upregulated in TGFβ3 treated chondronoids (FIG. 6D, E). In line with this, the ERG target and articular cartilage ECM gene PRG4 (Otha et al., 2015) is also highly upregulated in TGFβ3 treated pellets (FIG. 6B, C). Other ETS transcription factors, ELK3 and ERF are upregulated and highly expressed (FIG. 6D, E). During development Elk3 is expressed in mouse pre-cartilage condensations and expression is later restricted to perichondrium and future articular regions (Ayad et al., 2001) but a function in articular cartilage has not been described. ERF is expressed in osteoblasts and is important for normal bone development (Raouf and Seth, 2000), but again, a role in articular cartilage has not been identified. Another highly expressed and highly upregulated TF is TRPS1 (FIG. 6D, E). TRPS1 mutations cause a skeletal dysplasia, tricho-rhino-phalangeal syndrome, and Trps1 mutant mice have similar craniofacial defects, premature growth plate closure and defective long bone growth (Napierala et al., 2008). These features highlight TRPS1 function in growth plate chondrocytes, and although Trps1 is expressed in developing joints (Napierala et al., 2008), its role there has not been defined. Similarly, PLAGLI, highly expressed and upregulated in our TGFβ3-treated data set, is abundant in chondrogenic tissues during development (Tsuda et al., 2004), the KO mouse has delayed ossification (Varrault et al., 2006), but a role in articular cartilage isn't documented. Interestingly, MEOX1 and MEOX2, which are not expressed at day 48 of chondrogenic differentiation without TGFβ3 are highly upregulated with TGFβ3 (FIG. 6D, E, Supplementary table 1). Their role in articular cartilage development is uncharacterized, although their earlier role in somite development is well known (Reijntjes et al., 2007). BHLHE40, highly expressed and upregulated with TGFβ3 (FIG. 6D, Supplementary table 1), is a key molecular clock gene, again emphasizing the central role of circadian rhythm in cartilage homeostasis (Gonçalves and Meng, 2019; Gossan et al., 2013). The most highly expressed TF in TGFβ3 treated chondronoids, AEBP1, is also strongly upregulated (FIG. 6D, E). Alternative AEBP1 splicing produces mRNA encoding the transcription factor AEBP1 and mRNA encoding ACLP, a secreted core matrisome protein that binds collagens (Blackburn et al., 2018). AEBP1 mutations cause a form of Ehlers-Danlos syndrome with collagen fibril abnormalities (Blackburn et al., 2018; Vishwanath et al., 2020) and so it seems likely that AEBP1 upregulation in articular cartilage relates predominantly to its extracellular role.

Among the most strongly downregulated TFs in TGFβ3 treated chondronoids are the well-known TFs involved in chondrocyte hypertrophy, MEF2C, RUNX2, SP7, and DLX5 (FIG. 6E). NR3C1, with a known role in hypertrophy, is downregulated along with others we identified in the hypertrophic pathway, including, KLF2, KLF5, KLF10, DDIT3, DLX3, IRX5, ZNF277, and MXI1 (Supplementary table 1). Together, the core matrisome and transcription factor expression patterns reflect what is known about expression in growth plate and articular cartilage and reveal previously unknown expression changes that could be important in tissue development and homeostasis.

### Example 7. Hypertrophic Chondrocyte to Osteoblast Transdifferentiation

The fate of hypertrophic chondrocytes during endochondral ossification was long thought to involve cell death, followed by hypertrophic cartilage remodeling and vascular invasion; however, it has now become clear that an alternative fate is transdifferentiation to osteoblasts (Haseeb et al., 2021; Park et al., 2015; Tsang et al., 2014; Yang et al., 2014; Zhou et al., 2014). The ability of stem-cell derived chondrocytes to enter the transdifferentiation pathway in vitro has not been studied and so we do not know if the complex regulatory pathways and environmental cues are sufficiently recapitulated in these systems. This capacity is important for modelling human diseases that affect endochondral pathways at different stages of cartilage and bone development. To study osteoblast dysfunction in the context of endochondral bone formation, it is essential.

Although the regulatory mechanisms that drive transdifferentiation are not fully understood, some key components have been identified including RUNX2 (Qin et al., 2020; Wang et al., 2017; Xing et al., 2019), PTPN11 (Wang et al., 2017), and SP7 (Xing et al., 2019) expression in hypertrophic chondrocytes. Conversely, persistent SOX9 expression suppresses chondrocyte transdifferentiation to osteoblasts (Lui et al., 2019). While it is doubtless true that other important regulators will be identified over time, the strong RUNX2, SP7 and PTPN11 mRNA expression and reduced SOX9 expression in our hypertrophic chondrocytes (FIG. 3, FIG. 3.1) suggests they may be primed for transdifferentiation if the correct cues were provided in vitro.

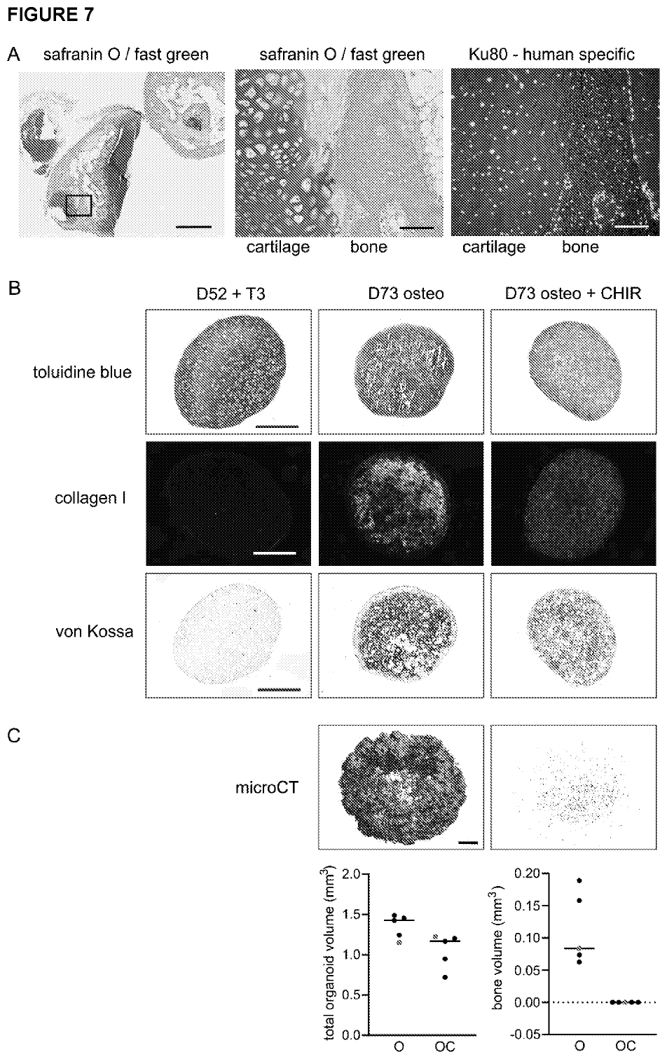

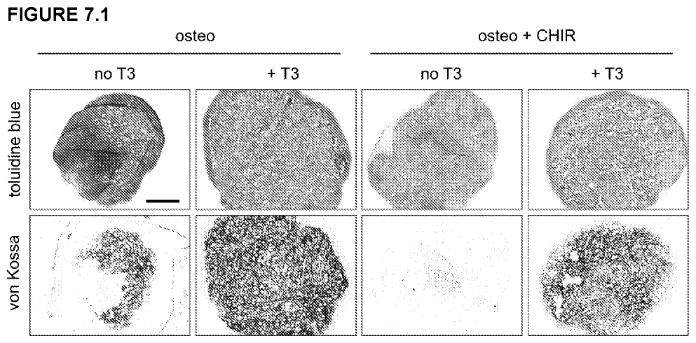

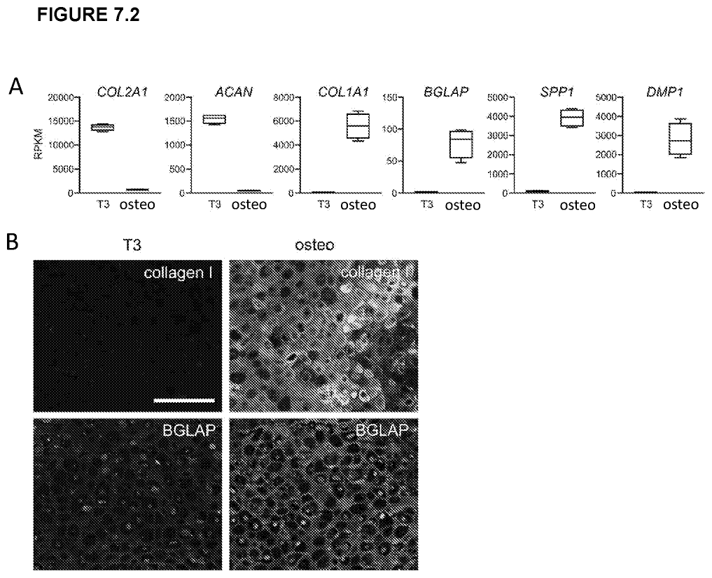

To test if our iPSC-derived chondrocytes could transdifferentiate into osteoblasts we first transplanted hypertrophic cartilage organoids subcutaneously into immunodeficient mice. The organoids were harvested after 13 weeks and analyzed histologically with safranin O/fast green staining for cartilage and bone respectively (FIG. 7A). The implants contain some residual cartilage with the characteristic hypertrophic chondrocytes embedded in the safranin O positive ECM; however, much of the implant has formed histologically recognizable bone (FIG. 7A). To discriminate human iPSC-derived cells from mouse cells that could have migrated into the implants we immunostained with a human-specific Ku80 antibody. The staining shows that the bone in the implant was derived from human cells (FIG. 7A), clearly indicating that the in vitro differentiated hypertrophic chondrocytes can transdifferentiate into bone cells when provided with signaling cues in vivo.

Next, we tested if providing culture conditions that support osteogenesis would allow transdifferentiation and bone formation in vitro. β-catenin plays a key role in facilitating chondrocyte transdifferentiation in mice (Jing et al., 2018), and so we cultured MCRIi018-B hypertrophic organoids in conventional osteogenic medium for three weeks, or in osteogenic medium with a 7-day pulse of CHIR99021 to activate the Wnt/β-catenin pathway followed by 2 weeks in osteogenic medium alone. We first compared the two osteogenic conditions using histology. There was a marked reduction in toluidine blue staining in CHIR99021 treated organoids indicating that aggrecan degradation had occurred in this osteogenic condition (FIG. 7B). This is consistent with the cartilage matrix remodeling that occurs during endochondral ossification in vivo. Collagen I, the major collagen type in bone, was deposited into the ECM in both osteogenic conditions, but the staining was more intense in organoids grown in osteogenic medium alone (FIG. 7B). We used von Kossa staining, commonly used to indicate matrix mineralization, to compare the osteogenic differentiation conditions. Again, while both osteogenic conditions induced matrix mineralization, the staining was more intense in organoids differentiated in osteogenic medium alone (FIG. 7B). Further, μCT analysis confirmed extensive deposition of a calcium phosphate mineral in organoids cultured in osteogenic medium alone suggesting transition to a bone-like organoid (FIG. 7B). By contrast, there was little μCT attenuation in organoids cultured in osteogenic medium with a CHIR99021 pulse even though these organoids showed positive von Kossa staining (FIG. 7B). Although these findings appear to conflict, the von Kossa stain detects phosphate and doesn't necessarily indicate the presence of calcium or hydroxyapatite, the calcium phosphate component of bone (Bonewald et al., 2003). This emphasizes that von Kossa staining should be interpreted with caution when assessing mineralized ECM.

We compared osteogenic differentiation conditions in a second iPSC line, MCRIi001-A-2, and confirmed that aggrecan degradation was more extensive in osteogenic conditions with a CHIR99021 pulse, and von Kossa staining was more intense in organoids cultured in osteogenic medium alone (FIG. 7.1). To determine if enhancing chondrocyte hypertrophy with T3 was important for transition to osteoblast-like cells we compared osteogenic differentiation in organoids that had been pre-treated with T3 with those without T3 pre-treatment. In both osteogenic conditions there was more extensive and more intense von Kossa staining in organoids pre-treated with T3 (FIG. 7.1), confirming that T3 treatment is essential for optimal transition to bone-like organoids.

Next, we examined gene expression changes during culture in osteogenic conditions and looked first at some well recognized, characteristic cartilage and osteoblast genes. (FIG. 7.2). Osteogenic conditions lead to a striking downregulation of hypertrophic cartilage genes, COL2A1 and ACAN, concomitant with a strong upregulation of the of the key osteoblast marker genes, COL1A1, BGLAP, SPP1 and DMP1 (FIG. 7.2A). The extracellular deposition of bone collagen I (COL1A1) and osteocalcin (BGLAP) protein under osteogenic conditions was confirmed by immunohistochemistry (FIG. 7.2B).

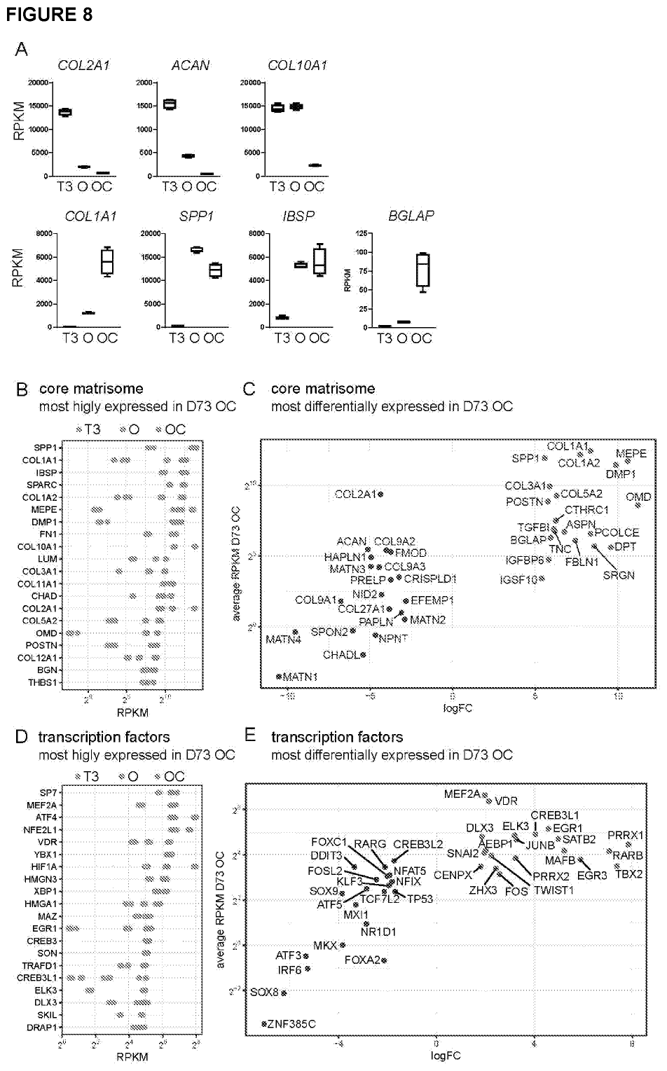

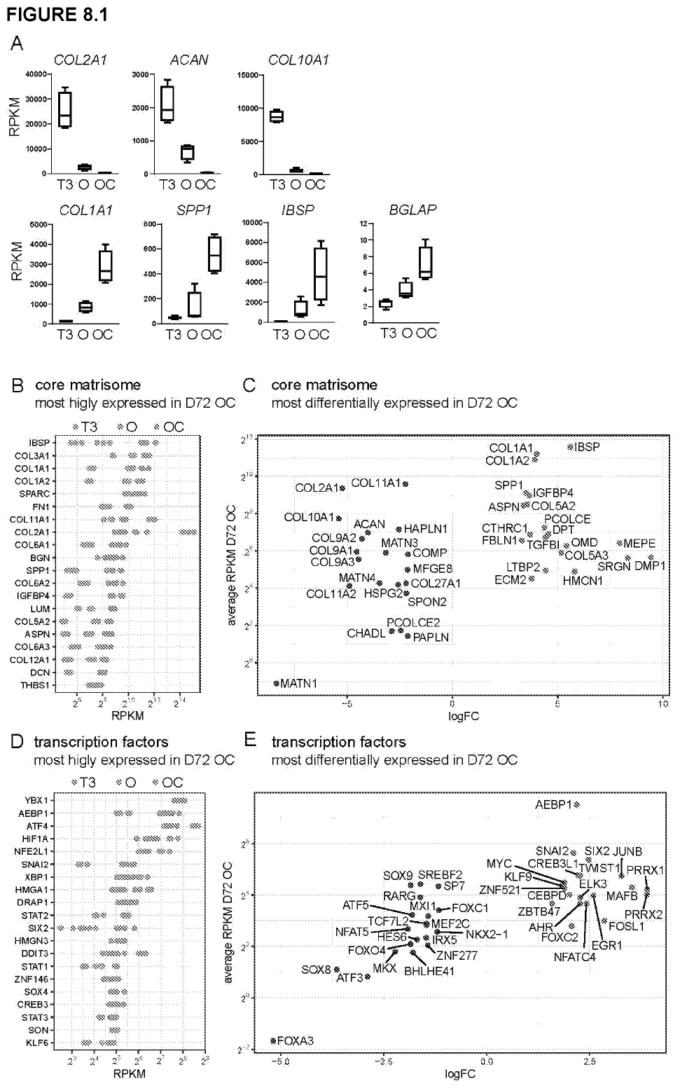

While both osteogenic conditions led to a striking downregulation of hypertrophic cartilage genes, COL2A1 and ACAN, but COL10A1 was only reduced in osteogenic medium with a CHIR99021 pulse (FIG. 8A). SPP1 and IBSP, highly expressed in osteoblasts, were upregulated in both osteogenic conditions, as was the main bone collagen, COL1A1, but COL1A1 expression was much higher in the osteogenic medium with a CHIR pulse (FIG. 8A). Other cartilage markers such as, SOX9, COMP, IHH, MATN3, CEBPB, and cartilage collagens, COL11A1, COL11A2, COL9A1, COL9A2, and COL9A3 were also downregulated more in osteogenic medium with a CHIR pulse, and bone markers, COL3A1, COL5A1, and OMD, were more highly expressed. We saw a similar pattern of gene expression changes in a second iPSC line, MCRIi019-A (FIG. 8.1). Based on this initial examination suggesting that the transition from hypertrophic chondrocytes to osteoblast-like cells was most efficient in osteogenic conditions with a CHIR pulse than in osteogenic medium alone, we focused further analyses on gene expression in osteogenic medium with a CHIR pulse.

Like chondrocytes, pre-osteoblasts and osteoblasts express a characteristic set of extracellular matrix proteins that includes collagens I (COL1A1 and COL1A2), III (COL3A1) and V (COL5A1 and COL5A2), proteoglycans, biglycan (BGN), decorin (DCN), keratocan (KERA) and asporin (ASPN), glycoproteins, osteonectin (SPARC) and thrombospondin 1 (THBS1), members of the SIBLING protein family, IBSP, SPP1, DMP1 and MEPE, and osteocalcin (BGLAP) (Lin et al., 2020). This pattern of gene expression changes was again similar when a second iPSC line, MCRIi019-A, was induced to transition to osteoblasts (FIG. 8.1).

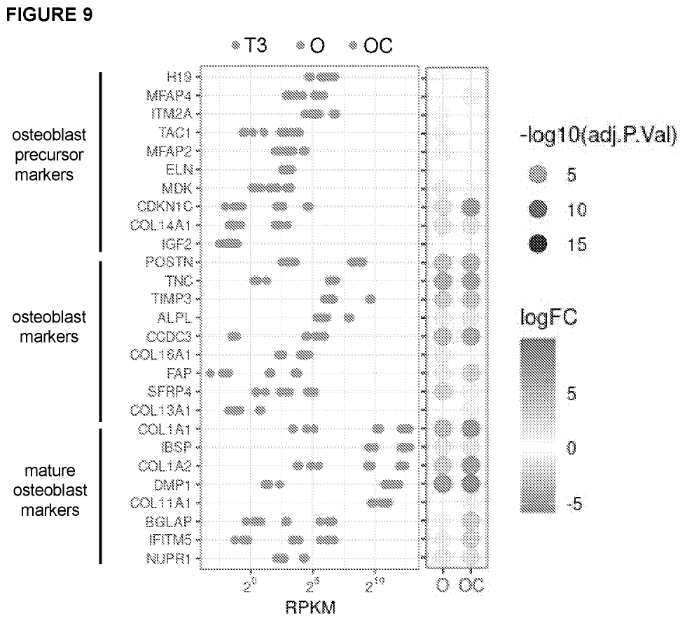

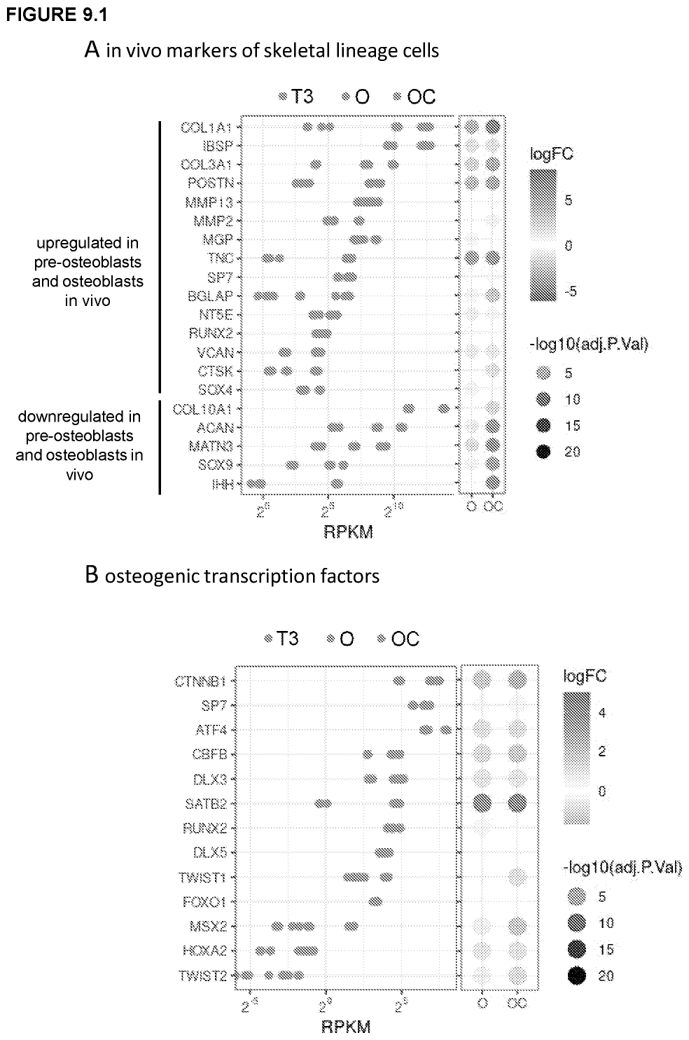

To provide further support for in vitro hypertrophic chondrocyte to pre-osteoblast/osteoblast transition we compared gene expression in our differentiation with expression of the top markers that define osteoblast precursor, osteoblast and more mature osteoblast cell clusters in single cell RNAseq of cells isolated from mouse bone (Ayturk et al., 2020). Osteoblast precursor marker genes are not greatly changed between T3 treated hypertrophic chondrocytes and organoids cultured in osteogenic conditions (FIG. 9). By contrast, many osteoblast and mature osteoblast marker genes are highly upregulated in osteogenic conditions. For example, osteoblast markers POSTN, TNC, CCDC3, FAP and SFRP4 are upregulated 54, 72, 121, 64 and 4-fold respectively in organoids cultured in osteogenic conditions with a CHIR pulse when compared to hypertrophic chondrocytes (FIG. 9). The mature osteoblast markers COL1A1, COL1A2, IBSP, DMP1 and IFITM5 are upregulated 324, 210, 7, 935 and 105-fold respectively in osteogenic conditions with CHIR and these 5 genes are the most highly expressed of all the marker genes in our dataset (FIG. 9). The gene expression data are consistent with the proposal that hypertrophic chondrocytes are transitioning to osteoblasts in vitro.

We next looked at expression of nineteen genes that mark hypertrophic chondrocyte, pre-osteoblast and osteoblast cell clusters during mouse in vivo transdifferentiation and skeletal development (Haseeb et al., 2021). In osteogenic conditions all but two genes, MGP and SOX4, follow the expression pattern identified during in vivo transdifferentiation (FIG. 8.2A). Of the 11 genes upregulated during transition to osteoblasts, nine are more highly expressed in organoids exposed to a CHIR pulse, and all five hypertrophic chondrocyte marker genes are more downregulated. In addition, all thirteen well characterized and well recognized osteogenic transcription factors (Chan et al., 2021) are expressed in organoids cultured in osteogenic conditions and 12 of them are more highly expressed than in hypertrophic chondrocytes (FIG. 8.2B).

This gene expression data suggests that our in vitro transdifferentiation protocol is recapitulating in vivo development and presents an opportunity to further understand how hypertrophic chondrocyte to osteoblast transdifferentiation is regulated. The twenty most highly expressed transcription factors (TFs) in MCRIi018-B osteoblasts include 11 with known roles in osteoblast differentiation or bone development, SP7, MEF2A, ATF4, NFE2L1, VDR, HIF1A, XBP1, EGR1, CREB3LI, DLX3 and SKIL (FIG. 8D) (Chan et al., 2021; Kim et al., 2010; Leupin et al., 2007; Pang et al., 2020; Price et al., 1998; Symoens et al., 2013; Tohmonda et al., 2011; Zhang et al., 2020). Other highly expressed TFs such as YBX1, HMGN3, HMGA1, MAZ, CREB3, SON, TRAFD1, ELK3 and DRAP1, have not been implicated in osteoblast differentiation but their high expression suggests they could be important. Similarly, the 20 most highly upregulated TFs include many with known roles in osteoblasts and bone, PRRX1, SATB2, RARB, TBX2, MAFB, PRRX2, TWIST1, FOS, JUNB, ZHX3 (FIG. 8E) (Chan et al., 2021; Dobreva et al., 2006; Liu et al., 2018, 2018; Suehiro et al., 2011; ten Berge et al., 1998; Wagner, 2002; Zankl et al., 2012) but others such as EGR3, CENPX, SNAI2 and ELK3 have unknown osteogenic roles. Our data also show that that this hypertrophic chondrocyte to osteoblast differentiation protocol is reproducible; ten of the 20 most highly expressed TFs in MCRIi018-B osteoblasts are also among the most highly expressed in MCRIi019-A osteoblasts (FIG. 8D, FIG. 8.1D) and similarly, 10 of the 20 most highly upregulated TFs are the same in osteoblasts derived from both iPSC lines (FIG. 8E, FIG. 8.1E).

Our data showing distinct core matrisome expression patterns in articular chondrocytes, hypertrophic chondrocytes, and osteoblasts, supports recent evidence showing that expression of core matrisome genes is sufficient to cluster scRNAseq data because each cell type produces a unique extracellular matrix (Sacher et al., 2021). Together, the mRNA expression signatures of core matrisome and transcription factor genes indicates that, when provided with culture conditions that support osteogenesis, hypertrophic chondrocytes can transition to pre-osteoblasts/osteoblasts allowing endochondral ossification to be modelled in vitro.

### Example 8-Disease Modeling Using Chondrocyte and Osteoblast Organoids Differentiated from Human iPSC

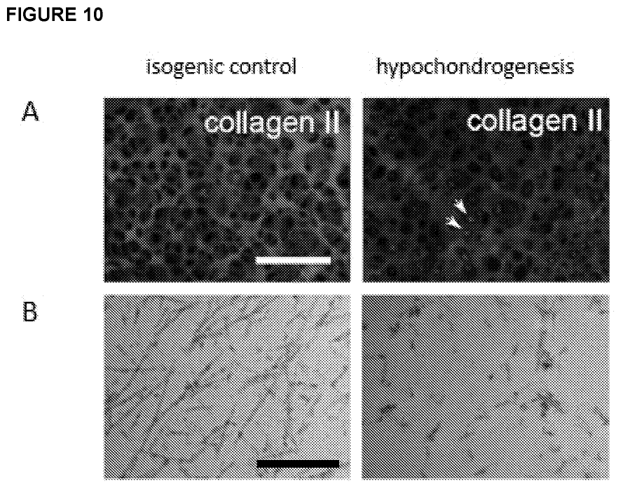

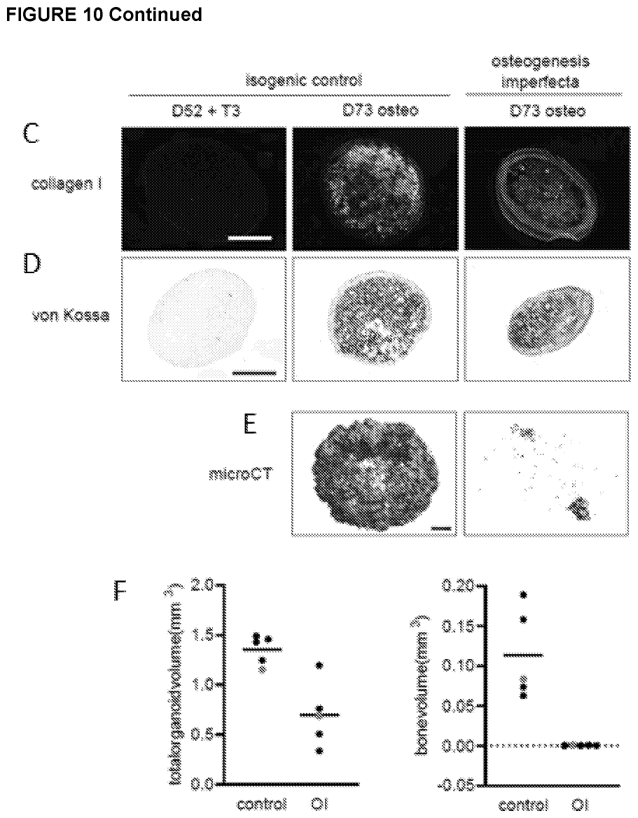

Using gene-edited iPSC lines with an inherited cartilage disease (hypochondrogenesis) COL2A1 p.G1113C mutation and isogenic control (Lilianty J, Bateman J F, Lamandé SR. Stem Cell Res. 2021 Aug. 25; 56:102515). iPSC were differentiated into mature chondrocytes using the methods described in the materials and methods and Example 3. The collagen II extracellular matrix was assessed by collagen II immunohistochemistry (FIG. 10A) and by electron microscopy (FIG. 10B). Both methods demonstrated the reduced collagen II extracellular matrix in the hypochondrogenesis mutant.

Using gene-edited iPSC lines with an inherited osteogenesis imperfecta, COL1A1 p.W1312C mutation, and isogenic control (Howden S, et al., Stem Cell Res. 2019 July; 38:101453) iPSC were differentiated into osteoblasts in vitro using the methods described in the materials and methods and Example 3. The osteogenesis imperfecta iPSC-derived osteoblasts show reduced collagen I extracellular matrix formation by immunohistochemistry (FIG. 10C), and reduced calcification by von Kossa staining (FIG. 10D). The reduced calcification (bone formation) of the osteogenesis imperfecta bone organoid is more dramatically shown by microCT analysis (FIG. 10E).

### Discussion

We have developed an iPSC differentiation protocol that follows normal mesoderm developmental pathways to produce sclerotome, chondrocyte progenitors then articular chondrocytes, or alternatively, chondrocytes that mature to hypertrophic chondrocytes and can transition to osteoblasts and produce a mineralized extracellular matrix. Our protocol thus recapitulates the key steps in growth plate development and endochondral ossification in vitro. At important stages we have documented global gene expression and identified unique gene expression patterns for extracellular matrix genes and transcription factors. These gene expression patterns mirror what is known about in vivo gene expression and can provide new insights into how cartilage development and endochondral bone growth are regulated.

We first optimized post-sclerotome chondrogenesis. Our optimized protocol includes forming pellets at the end of day 4, which is 24 hours after beginning somitic mesoderm to sclerotome differentiation, to provide cells with the 3D environment essential for chondrocytes to maintain their phenotype (Ecke et al., 2019). We found differentiation was more uniform in iPSC lines maintained in feeder free culture. An innovation introduced that has not been reported for chondrocyte differentiation is using orbital rotary culture, with multiple pellets in non-adherent dishes. In addition to reducing the time and precision needed to maintain pellets compared to individual pellet culture, we found that rotary culture influenced differentiation rate and specificity. Chondrocytes matured towards hypertrophy and expressed the hypertrophic cartilage specific collagen X most efficiently when pellets were transferred to rotary culture between day 13 and day 20, depending on the cell line. By day 48 pellets transferred to rotary culture between day 13 and day 27 had no obvious non-cartilage cells within the pellets or surrounding the pellets making them an ideal tool for investigating chondrocyte maturation and hypertrophy and use in biomaterials for therapeutic use.

Post-sclerotome, an FGF2 pulse and long-term culture supplemented with TGFβ3 produces articular chondrocytes that express the sentinel articular chondrocyte marker PRG4 as well as a range of other articular cartilage extracellular matrix proteins such as CILP, ASPN, COL1A1, COL3A1, and elastic fiber components, consistent with what is known about the articular cartilage matrisome.

The alternative post-sclerotome cell fate, growth plate hypertrophic chondrocytes, can be induced with an FGF2 pulse followed by culture in our chondrogenic basal medium APEL2. Some hypertrophic markers such as COL10A1, RUNX2, SPP1, SP7, MMP13, IHH, and MEF2C are upregulated with spontaneous hypertrophy and are therefore likely to be early hypertrophy markers, while others including ALPL, FOSL2, VEGFA, BMP7 and DMP1 are only upregulated after stimulation with T3 (triiodothyronine). T3 is required to downregulate the chondrogenic transcription factors SOX9/SOX5/SOX6 and cartilage matrisome genes COL9A1, COMP and MATN1. In mouse skeletal development, T3 is required to promote hypertrophy around the tibial secondary ossification centers and allow hypertrophic chondrocyte to osteoblast transition (Aghajanian et al., 2017). Others have used T3 to promote hypertrophy in iPSC chondrocyte differentiation (Pretemer et al., 2021); however, that protocol did not follow normal post-sclerotome developmental pathways and included sequential and overlapping dexamethasone, PDGF, TGFβ3, BMP4, T3 and β-glycerophosphate addition. Our gene expression data show our differentiation program from chondroprogenitors to chondrocytes to chondrocyte hypertrophy follow what is known about matrisome and transcription factor expression during growth plate maturation.

Our transcriptome data suggest that the hypertrophic chondrocytes are primed for transdifferentiation to osteoblasts. Hypertrophic cartilage core matrisome components COL10A1, SPP1 and DMP1 are upregulated, and genes in angiogenic pathways such as VEGFA that promote blood vessel invasion into the hypertrophic cartilage, and genes in TGFβ and WNT signaling pathways are differentially expressed. Importantly, we show that both HIF-1a (HIF1A) and HIF-2a (EPAS1) are upregulated during hypertrophic differentiation in our chondronoid in vitro model consistent with the hypoxic/angiogenic switch required in the normal in vivo endochondral ossification process (Liu and Olsen, 2014; Schipani et al., 2009).

Building on this, we show that hypertrophic chondrocytes can transition to osteoblasts in vitro. When hypertrophic chondrocytes are provided with factors that promote osteogenesis, β-glycerophosphate, ascorbic acid and dexamethasone, they switch on and/or upregulate characteristic osteoblast matrisome and transcription factor genes and downregulate cartilage specific gene networks. The dynamic gene expression changes during this in vitro transition mirror the documented changes during in vivo hypertrophic chondrocyte to osteoblast transdifferention in mice (Haseeb et al., 2021), and expression in osteoblast lineage cells in mouse bone (Ayturk et al., 2020). In osteogenic conditions organoids produce a collagen I extracellular matrix and deposit calcium phosphate mineral consistent with transition to a bone-like organoid. Importantly, we found that promoting hypertrophy with T3 prior to osteogenic culture was critical for matrix mineralization.

We have established an iPSC differentiation protocol that generates hypertrophic chondrocytes with the plasticity for onward transition to osteoblasts thus recapitulating endochondral ossification in vitro. Using a robust, reproducible method to first generate sclerotome, then minimal growth factor stimulation to promote chondrocyte maturation means that the chondrocytes follow in vivo sequential developmental pathways making the system ideal for exploring how development is regulated and modelling genetic skeletal disorders. Stimulating in vitro hypertrophic chondrocyte transdifferentiation to osteoblasts is an important and significant advance that will enhance our understanding of this fundamental but newly recognized pathway and allow in vitro bone disease modelling. Choosing the alternative differentiation pathway from chondroprogenitors to articular cartilage enables arthritis related studies and production of cartilage biomaterials for tissue regeneration. Our protocol is reproducible in all the cell lines we have tested but an important finding is that the rate of post-sclerotome differentiation and maturation varies slightly between cell lines and this produces significant gene expression changes. This emphasizes that for disease modelling it is critical to minimize inter-cell line variability and compare isogenic control and mutant lines.

### REFERENCES

Adkar S S, Wu C-L, Willard V P, Dicks A, Ettyreddy A, Steward N, Bhutani N, Gersbach C A, Guilak F. 2019. Step-Wise Chondrogenesis of Human Induced Pluripotent Stem Cells and Purification Via a Reporter Allele Generated by CRISPR-Cas9 Genome Editing. Stem Cells 37:65-76. doi: 10.1002/stem.2931
Aghajanian P, Mohan S. 2018. The art of building bone: emerging role of chondrocyte-to-osteoblast transdifferentiation in endochondral ossification. Bone Res 6:1-9. doi: 10.1038/s41413-018-0021-z
Aghajanian P, Xing W, Cheng S, Mohan S. 2017. Epiphyseal bone formation occurs via thyroid hormone regulation of chondrocyte to osteoblast transdifferentiation. Sci Rep 7:10432. doi: 10.1038/s41598-017-11050-1
Ayadi A, Suelves M, Dollé P, Wasylyk B. 2001. Net, an Ets ternary complex transcription factor, is expressed in sites of vasculogenesis, angiogenesis, and chondrogenesis during mouse development. Mechanisms of Development 102:205-208. doi: 10.1016/S0925-4773 (01) 00289-1
Ayturk U M, Scollan J P, Goz Ayturk D, Suh E S, Vesprey A, Jacobsen C M, Divieti Pajevic P, Warman M L. 2020. Single-Cell RNA Sequencing of Calvarial and Long-Bone Endocortical Cells. J Bone Miner Res 35:1981-1991. doi: 10.1002/jbmr.4052
Blackburn P R, Xu Z, Tumelty K E, Zhao R W, Monis W J, Harris K G, Gass J M, Cousin M A, Boczek N J, Mitkov M V, Cappel M A, Francomano C A, Parisi J E, Klee E W, Faqeih E, Alkuraya F S, Layne M D, McDonnell N B, Atwal P S. 2018. Bi-allelic Alterations in AEBP1 Lead to Defective Collagen Assembly and Connective Tissue Structure Resulting in a Variant of Ehlers-Danlos Syndrome. The American Journal of Human Genetics 102:696-705. doi: 10.1016/j.ajhg.2018.02.018
Bolger A M, Lohse M, Usadel B. 2014. Trimmomatic: a flexible trimmer for Illumina sequence data. Bioinformatics 30:2114-2120. doi: 10.1093/bioinformatics/btu170
Bonafe L, Cormier-Daire V, Hall C, Lachman R, Mortier G, Mundlos S, Nishimura G, Sangiorgi L, Savarirayan R, Sillence D, Spranger J, Superti-Furga A, Warman M, Unger S. 2015. Nosology and classification of genetic skeletal disorders: 2015 revision. Am J Med Genet A 167A: 2869-2892. doi: 10.1002/ajmg.a.37365
Bonewald L F, Harris S E, Rosser J, Dallas M R, Dallas S L, Camacho N P, Boyan B, Boskey A. 2003. von Kossa staining alone is not sufficient to confirm that mineralization in vitro represents bone formation. Calcif Tissue Int 72:537-547. doi: 10.1007/s00223-002-1057-y
Boyanich R, Becker T, Chen F, Kirk T B, Allison G, Wu J-P. 2019. Application of confocal, SHG and atomic force microscopy for characterizing the structure of the most superficial layer of articular cartilage. J Microsc 275:159-171. doi: 10.1111/jmi.12824
Cameron T L, Gresshoff I L, Bell K M, Piróg K A, Sampurno L, Hartley C L, Sanford E M, Wilson R, Ermann J, Boot-Handford R P, Glimcher L H, Briggs M D, Bateman J F. 2015. Cartilage-specific ablation of XBP1 signaling in mouse results in a chondrodysplasia characterized by reduced chondrocyte proliferation and delayed cartilage maturation and mineralization. Osteoarthritis Cartilage 23:661-670. doi: 10.1016/j.joca.2015.01.001
Chan W C W, Tan Z, To M K T, Chan D. 2021. Regulation and Role of Transcription Factors in Osteogenesis. International Journal of Molecular Sciences 22:5445. doi: 10.3390/ijms22115445
Cleary M A, van Osch GJVM, Brama P A, Hellingman C A, Narcisi R. 2015. FGF, TGFβ and Wnt crosstalk: embryonic to in vitro cartilage development from mesenchymal stem cells. J Tissue Eng Regen Med 9:332-342. doi: 10.1002/term.1744
Craft A M, Ahmed N, Rockel J S, Baht G S, Alman B A, Kandel R A, Grigoriadis A E, Keller G M. 2013. Specification of chondrocytes and cartilage tissues from embryonic stem cells. Development 140:2597-2610. doi: 10.1242/dev.087890
Craft A M, Rockel J S, Nartiss Y, Kandel R A, Alman B A, Keller G M. 2015. Generation of articular chondrocytes from human pluripotent stem cells. Nat Biotechnol 33:638-645. doi: 10.1038/nbt.3210
Decker R S, Koyama E, Pacifici M. 2015. Articular Cartilage: Structural and Developmental Intricacies and Questions. Curr Osteoporos Rep 13:407-414. doi: 10.1007/s11914-015-0290-z
Decker R S, Koyama E, Pacifici M. 2014. Genesis and morphogenesis of limb synovial joints and articular cartilage. Matrix Biol 39:5-10. doi: 10.1016/j.matbio.2014.08.006
Della Bella E, Buetti-Dinh A, Licandro G, Ahmad P, Basoli V, Alini M, Stoddart M J. 2021. Dexamethasone Induces Changes in Osteogenic Differentiation of Human Mesenchymal Stromal Cells via SOX9 and PPARG, but Not RUNX2. International Journal of Molecular Sciences 22:4785. doi: 10.3390/ijms22094785
Diaz-Romero J, Nesic D. 2017. S100A1 and S100B: Calcium Sensors at the Cross-Roads of Multiple Chondrogenic Pathways. Journal of Cellular Physiology 232:1979-1987. doi: https://doi.org/10.1002/jcp.25720
Diegel C R, Hann S, Ayturk U M, Hu J C W, Lim K, Droscha C J, Madaj Z B, Foxa G E, Izaguirre I, Core V V and T, Paracha N, Pidhaynyy B, Dowd T L, Robling A G, Warman M L, Williams
B O. 2020. An osteocalcin-deficient mouse strain without endocrine abnormalities. PLOS Genetics 16: e1008361. doi: 10.1371/journal.pgen.1008361
Dobin A, Davis C A, Schlesinger F, Drenkow J, Zaleski C, Jha S, Batut P, Chaisson M, Gingeras T R. 2013. STAR: ultrafast universal RNA-seq aligner. Bioinformatics 29:15-21. doi: 10.1093/bioinformatics/bts635
Dobreva G, Chahrour M, Dautzenberg M, Chirivella L, Kanzler B, Fariñas I, Karsenty G, Grosschedl R. 2006. SATB2 is a multifunctional determinant of craniofacial patterning and osteoblast differentiation. Cell 125:971-986. doi: 10.1016/j.cell.2006.05.012
Dreher S I, Fischer J, Walker T, Diederichs S, Richter W. 2020. Significance of MEF2C and RUNX3 Regulation for Endochondral Differentiation of Human Mesenchymal Progenitor Cells. Front Cell Dev Biol 8:81. doi: 10.3389/fcell.2020.00081
Ecke A, Lutter A-H, Scholka J, Hansch A, Becker R, Anderer U. 2019. Tissue Specific Differentiation of Human Chondrocytes Depends on Cell Microenvironment and Serum Selection. Cells 8. doi: 10.3390/cells8080934
Ewels P, Magnusson M, Lundin S, Käller M. 2016. MultiQC: summarize analysis results for multiple tools and samples in a single report. Bioinformatics 32:3047-3048. doi: 10.1093/bioinformatics/btw354
Fujita K, Xing Q, Khosla S, Monroe D G. 2014. Mutual Enhancement of Differentiation of Osteoblasts and Osteocytes Occurs Through Direct Cell-Cell Contact. Journal of Cellular Biochemistry 115:2039-2044. doi: 10.1002/jcb.24880
Gonçalves C F, Meng Q-J. 2019. Timing metabolism in cartilage and bone: links between circadian clocks and tissue homeostasis. J Endocrinol. doi: 10.1530/JOE-19-0256
Gossan N, Zeef L, Hensman J, Hughes A, Bateman J F, Rowley L, Little C B, Piggins H D, Rattray M, Boot-Handford R P, Meng Q-J. 2013. The circadian clock in murine chondrocytes regulates genes controlling key aspects of cartilage homeostasis. Arthritis Rheum 65:2334-2345. doi: 10.1002/art.38035
Haseeb A, Kc R, Angelozzi M, de Charleroy C, Rux D, Tower R J, Yao L, Pellegrino da Silva R, Pacifici M, Qin L, Lefebvre V. 2021. SOX9 keeps growth plates and articular cartilage healthy by inhibiting chondrocyte dedifferentiation/osteoblastic redifferentiation. Proc Natl Acad Sci U S A 118. doi: 10.1073/pnas.2019152118
Hata K, Takahata Y, Murakami T, Nishimura R. 2017. Transcriptional Network Controlling Endochondral Ossification. J Bone Metab 24:75-82. doi: 10.11005/jbm.2017.24.2.75
Hirao M, Tamai N, Tsumaki N, Yoshikawa H, Myoui A. 2006. Oxygen Tension Regulates Chondrocyte Differentiation and Function during Endochondral Ossification*. Journal of Biological Chemistry 281:31079-31092. doi: 10.1074/jbc.M602296200
Houben A, Kostanova-Poliakova D, Weissenböck M, Graf J, Teufel S, von der Mark K, Hartmann C. 2016. β-catenin activity in late hypertrophic chondrocytes locally orchestrates osteoblastogenesis and osteoclastogenesis. Development 143:3826-3838. doi: 10.1242/dev.137489
Howden S, Hosseini Far H, Motazedian A, Elefanty A G, Stanley E G, Lamandé S R, Bateman J F. 2019. The use of simultaneous reprogramming and gene correction to generate an osteogenesis imperfecta patient COL1A1 c. 3936 G>T iPSC line and an isogenic control iPSC line. Stem Cell Res 38:101453. doi: 10.1016/j.scr.2019.101453
Iwamoto M, Tamamura Y, Koyama E, Komori T, Takeshita N, Williams J A, Nakamura T, Enomoto-Iwamoto M, Pacifici M. 2007. Transcription factor ERG and joint and articular cartilage formation during mouse limb and spine skeletogenesis. Dev Biol 305:40-51. doi: 10.1016/j.ydbio.2007.01.037
Jing Y, Jing J, Wang K, Chan K, Harris S E, Hinton R J, Feng J Q. 2018. Vital Roles of β-catenin in Trans-differentiation of Chondrocytes to Bone Cells. Int J Biol Sci 14:1-9. doi: 10.7150/ijbs.23165
Kao T, Labonne T, Niclis J C, Chaurasia R, Lokmic Z, Qian E, Bruveris F F, Howden S E, Motazedian A, Schiesser J V, Costa M, Sourris K, Ng E, Anderson D, Giudice A, Farlie P, Cheung M, Lamande S R, Penington A J, Parish C L, Thomson L H, Rafii A, Elliott D A, Elefanty
A G, Stanley E G. 2016. GAPTrap: A Simple Expression System for Pluripotent Stem Cells and Their Derivatives. Stem Cell Reports 7:518-526. doi: 10.1016/j.stemcr.2016.07.015
Karsenty G, Kronenberg H M, Settembre C. 2009. Genetic control of bone formation. Annu Rev Cell Dev Biol 25:629-648. doi: 10.1146/annurev.cellbio.042308.113308
Kawata M, Mori D, Kanke K, Hojo H, Ohba S, Chung U-I, Yano F, Masaki H, Otsu M, Nakauchi H, Tanaka S, Saito T. 2019. Simple and Robust Differentiation of Human Pluripotent Stem Cells toward Chondrocytes by Two Small-Molecule Compounds. Stem Cell Reports 13:530-544. doi: 10.1016/j.stemcr.2019.07.012
Kim J, Xing W, Wergedal J, Chan J Y, Mohan S. 2010. Targeted disruption of nuclear factor erythroid-derived 2-like 1 in osteoblasts reduces bone size and bone formation in mice. Physiol Genomics 40:100-110. doi: 10.1152/physiolgenomics.00105.2009
Kronenberg H M. 2003. Developmental regulation of the growth plate. Nature 423:332-336. doi: 10.1038/nature01657
Kung L H W, Sampurno L, Yammine K M, Graham A, McDonald P, Bateman J F, Shoulders M D, Lamandé S R. 2020. CRISPR/Cas9 editing to generate a heterozygous COL2A1 p.G1170S human chondrodysplasia iPSC line, MCRIi019-A-2, in a control iPSC line, MCRIi019-A. Stem Cell Res 48:101962. doi: 10.1016/j.scr.2020.101962
Lambert S A, Jolma A, Campitelli L F, Das P K, Yin Y, Albu M, Chen X, Taipale J, Hughes T R, Weirauch M T. 2018. The Human Transcription Factors. Cell 172:650-665. doi: 10.1016/j.cell.2018.01.029
Law C W, Alhamdoosh M, Su S, Dong X, Tian L, Smyth G K, Ritchie M E. 2016. RNA-seq analysis is easy as 1-2-3 with limma, Glimma and edgeR. F1000Res 5. doi: 10.12688/f1000research.9005.3
Lee H-H, Chang C-C, Shieh M-J, Wang J-P, Chen Y-T, Young T-H, Hung S-C. 2013. Hypoxia Enhances Chondrogenesis and Prevents Terminal Differentiation through PI3K/Akt/FoxO Dependent Anti-Apoptotic Effect. Sci Rep 3. doi: 10.1038/srep02683
Leung V Y L, Gao B, Leung K K H, Melhado I G, Wynn S L, Au T Y K, Dung N W F, Lau J Y B, Mak A C Y, Chan D, Cheah K S E. 2011. SOX9 governs differentiation stage-specific gene expression in growth plate chondrocytes via direct concomitant transactivation and repression. PLOS Genet 7: e1002356. doi: 10.1371/journal.pgen.1002356
Leupin O, Kramer I, Collette N M, Loots G G, Natt F, Kneissel M, Keller H. 2007. Control of the SOST bone enhancer by PTH using MEF2 transcription factors. J Bone Miner Res 22:1957-1967. doi: 10.1359/jbmr.070804
Liao Y, Smyth G K, Shi W. 2014. featureCounts: an efficient general purpose program for assigning sequence reads to genomic features. Bioinformatics 30:923-930. doi: 10.1093/bioinformatics/btt656
Lilianty J, Bateman J F, Lamandé S R. Generation of a heterozygous COL2A1 (p.G1113C) hypochondrogenesis mutation iPSC line, MCRIi019-A-7, using CRISPR/Cas9 gene editing. Stem Cell Res. 2021 Aug. 25; 56:102515
Lin X, Patil S, Gao Y-G, Qian A. 2020. The Bone Extracellular Matrix in Bone Formation and Regeneration. Front Pharmacol 11. doi: 10.3389/fphar.2020.00757
Lin Y-C, Niceta M, Muto V, Vona B, Pagnamenta A T, Maroofian R, Beetz C, van Duyvenvoorde H, Dentici M L, Lauffer P, Vallian S, Ciolfi A, Pizzi S, Bauer P, Grüning N-M, Bellacchio E, Del Fattore A, Petrini S, Shaheen R, Tiosano D, Halloun R, Pode-Shakked B, Albayrak H M, Işik E, Wit J M, Dittrich M, Freire B L, Bertola D R, Jorge A A L, Barel O, Sabir A H, Al Tenaiji A M J, Taji S M, Al-Sannaa N, Al-Abdulwahed H, Digilio M C, Irving M, Anikster Y, Bhavani G S L, Girisha K M, Genomics England Research Consortium, Haaf T, Taylor J C, Dallapiccola B, Alkuraya F S, Yang R-B, Tartaglia M. 2021. SCUBE3 loss-of-function causes a recognizable recessive developmental disorder due to defective bone morphogenetic protein signaling. Am J Hum Genet 108:115-133. doi: 10.1016/j.ajhg.2020.11.015
Liu C-F, Samsa W E, Zhou G, Lefebvre V. 2017. Transcriptional control of chondrocyte specification and differentiation. Semin Cell Dev Biol 62:34-49. doi: 10.1016/j.semcdb.2016.10.004
Liu N, Schoch K, Luo X, Pena L D M, Bhavana V H, Kukolich M K, Stringer S, Powis Z, Radtke K, Mroske C, Deak K L, McDonald M T, McConkie-Rosell A, Markert M L, Kranz P G, Stong N, Need A C, Bick D, Amaral M D, Worthey E A, Levy S, Undiagnosed Diseases Network (UDN), Wangler M F, Bellen H J, Shashi V, Yamamoto S. 2018. Functional variants in TBX2 are associated with a syndromic cardiovascular and skeletal developmental disorder. Hum Mol Genet 27:2454-2465. doi: 10.1093/hmg/ddyl46
Liu Y, Olsen B R. 2014. Distinct VEGF functions during bone development and homeostasis. Arch Immunol Ther Exp (Warsz) 62:363-368. doi: 10.1007/s00005-014-0285-y
Loh K M, Chen A, Koh P W, Deng T Z, Sinha R, Tsai J M, Barkal A A, Shen K Y, Jain R, Morganti R M, Shyh-Chang N, Fernhoff N B, George B M, Wernig G, Salomon R E A, Chen Z, Vogel H, Epstein J A, Kundaje A, Talbot W S, Beachy P A, Ang L T, Weissman I L. 2016. Mapping the Pairwise Choices Leading from Pluripotency to Human Bone, Heart, and Other Mesoderm Cell Types. Cell 166:451-467. doi: 10.1016/j.cell.2016.06.011
Lui J C, Yue S, Lee A, Kikani B, Temnycky A, Barnes K M, Baron J. 2019. Persistent Sox9 expression in hypertrophic chondrocytes suppresses transdifferentiation into osteoblasts. Bone 125:169-177. doi: 10.1016/j.bone.2019.05.027
McIntosh B E, Brown M E, Duffin B M, Maufort J P, Vereide D T, Slukvin I I, Thomson J A. 2015. Nonirradiated NOD, B6.SCID I12rγ−/− Kit (W41/W41) (NBSGW) mice support multilineage engraftment of human hematopoietic cells. Stem Cell Reports 4:171-180. doi: 10.1016/j.stemcr.2014.12.005
Moriishi T, Ozasa R, Ishimoto T, Nakano T, Hasegawa T, Miyazaki T, Liu W, Fukuyama R, Wang Y, Komori H, Qin X, Amizuka N, Komori T. 2020. Osteocalcin is necessary for the alignment of apatite crystallites, but not glucose metabolism, testosterone synthesis, or muscle mass. PLOS Genetics 16: e1008586. doi: 10.1371/journal.pgen.1008586
Naba A, Clauser K R, Ding H, Whittaker C A, Carr S A, Hynes R O. 2016. The extracellular matrix: Tools and insights for the “omics” era. Matrix Biol 49:10-24. doi: 10.1016/j.matbio.2015.06.003
Nakajima T, Shibata M, Nishio M, Nagata S, Alev C, Sakurai H, Toguchida J, Ikeya M. 2018. Modeling human somite development and fibrodysplasia ossificans progressiva with induced pluripotent stem cells. Development 145. doi: 10.1242/dev.165431
Napierala D, Sam K, Morello R, Zheng Q, Munivez E, Shivdasani R A, Lee B. 2008. Uncoupling of chondrocyte differentiation and perichondrial mineralization underlies the skeletal dysplasia in tricho-rhino-phalangeal syndrome. Hum Mol Genet 17:2244-2254. doi: 10.1093/hmg/ddn125
Nur Patria Y, Lilianty J, Elefanty A G, Stanley E G, Labonne T, Bateman J F, Lamandé S R. 2020. Generation of a SOX9-tdTomato reporter human iPSC line, MCRIi001-A-2, using CRISPR/Cas9 editing. Stem Cell Res 42:101689. doi: 10.1016/j.scr.2019.101689
O'Connor S K, Katz D B, Oswald S J, Groneck L, Guilak F. 2020. Formation of Osteochondral Organoids from Murine Induced Pluripotent Stem Cells. Tissue Eng Part A. doi: 10.1089/ten.TEA.2020.0273
Ohta Y, Okabe T, Larmour C, Di Rocco A, Maijenburg M W, Phillips A, Speck N A, Wakitani S, Nakamura T, Yamada Y, Enomoto-Iwamoto M, Pacifici M, Iwamoto M. 2015. Articular cartilage endurance and resistance to osteoarthritic changes require transcription factor Erg. Arthritis Rheumatol 67:2679-2690. doi: 10.1002/art.39243
Okuma T, Hirata M, Yano F, Mori D, Kawaguchi H, Chung U, Tanaka S, Saito T. 2015. Regulation of mouse chondrocyte differentiation by CCAAT/enhancer-binding proteins. Biomed Res 36:21-29. doi: 10.2220/biomedres.36.21
Oldershaw R A, Baxter M A, Lowe E T, Bates N, Grady L M, Soncin F, Brison D R, Hardingham T E, Kimber S J. 2010. Directed differentiation of human embryonic stem cells toward chondrocytes. Nat Biotechnol 28:1187-1194. doi: 10.1038/nbt.1683
Pang L, Zhang Z, Shen Y, Cheng Z, Gao X, Zhang B, Wang X, Tian H. 2020. Mutant dlx3b disturbs normal tooth mineralization and bone formation in zebrafish. PeerJ 8: e8515. doi: 10.7717/peerj.8515
Park J, Gebhardt M, Golovchenko S, Perez-Branguli F, Hattori T, Hartmann C, Zhou X, deCrombrugghe B, Stock M, Schneider H, von der Mark K. 2015. Dual pathways to endochondral osteoblasts: a novel chondrocyte-derived osteoprogenitor cell identified in hypertrophic cartilage. Biol Open 4:608-621. doi: 10.1242/bio.201411031
Pogue R, Lyons K. 2006. BMP signaling in the cartilage growth plate. Curr Top Dev Biol 76:1-48. doi: 10.1016/S0070-2153 (06) 76001-X
Pretemer Y, Kawai S, Nagata S, Nishio M, Watanabe M, Tamaki S, Alev C, Yamanaka Y, Xue J-Y, Wang Z, Fukiage K, Tsukanaka M, Futami T, Ikegawa S, Toguchida J. 2021. Differentiation of Hypertrophic Chondrocytes from Human iPSCs for the In Vitro Modeling of Chondrodysplasias. Stem Cell Reports 16:610-625. doi: 10.1016/j.stemcr.2021.01.014
Price J A, Bowden D W, Wright J T, Pettenati M J, Hart T C. 1998. Identification of a mutation in DLX3 associated with tricho-dento-osseous (TDO) syndrome. Hum Mol Genet 7:563-569. doi: 10.1093/hmg/7.3.563
Qin X, Jiang Q, Nagano K, Moriishi T, Miyazaki T, Komori H, Ito K, Mark K von der, Sakane C, Kaneko H, Komori T. 2020. Runx2 is essential for the transdifferentiation of chondrocytes into osteoblasts. PLOS Genet 16: e1009169. doi: 10.1371/journal.pgen.1009169
Raouf A, Seth A. 2000. Ets transcription factors and targets in osteogenesis. Oncogene 19:6455-6463. doi: 10.1038/sj.onc.1204037
Reijntjes S, Stricker S, Mankoo B S. 2007. A comparative analysis of Meox1 and Meox2 in the developing somites and limbs of the chick embryo. Int J Dev Biol 51:753-759. doi: 10.1387/ijdb.072332sr
Robins J C, Akeno N, Mukherjee A, Dalal R R, Aronow B J, Koopman P, Clemens T L. 2005. Hypoxia induces chondrocyte-specific gene expression in mesenchymal cells in association with transcriptional activation of Sox9. Bone 37:313-322. doi: 10.1016/j.bone.2005.04.040
Sacher F, Feregrino C, Tschopp P, Ewald C Y. 2021. Extracellular matrix gene expression signatures as cell type and cell state identifiers. Matrix Biology Plus 100069. doi: 10.1016/j.mbplus.2021.100069
Sadedin S P, Pope B, Oshlack A. 2012. Bpipe: a tool for running and managing bioinformatics pipelines. Bioinformatics 28:1525-1526. doi: 10.1093/bioinformatics/bts167
Schipani E, Maes C, Carmeliet G, Semenza G L. 2009. Regulation of osteogenesis-angiogenesis coupling by HIFs and VEGF. J Bone Miner Res 24:1347-1353. doi: 10.1359/jbmr.090602
Shang J, Liu H, Li J, Zhou Y. 2014. Roles of hypoxia during the chondrogenic differentiation of mesenchymal stem cells. Curr Stem Cell Res Ther 9:141-147. doi: 10.2174/1574888x09666131230142459
Shimo T, Koyama E, Okui T, Masui M, Kunisada Y, Ibaragi S, Yoshioka N, Kurio N, Yoshida S, Sasaki A, Iwamoto M. 2019. Retinoic Receptor Signaling Regulates Hypertrophic Chondrocyte-specific Gene Expression. In Vivo 33:85-91. doi: 10.21873/invivo.11443
Soeda T, Deng J M, de Crombrugghe B, Behringer R R, Nakamura T, Akiyama H. 2010. Sox9-expressing precursors are the cellular origin of the cruciate ligament of the knee joint and the limb tendons. Genesis 48:635-644. doi: 10.1002/dvg.20667
Suehiro F, Nishimura M, Kawamoto T, Kanawa M, Yoshizawa Y, Murata H, Kato Y. 2011. Impact of zinc fingers and homeoboxes 3 on the regulation of mesenchymal stem cell osteogenic differentiation. Stem Cells Dev 20:1539-1547. doi: 10.1089/scd.2010.0279
Sueyoshi T, Yamamoto K, Akiyama H. 2012. Conditional deletion of Tgfbr2 in hypertrophic chondrocytes delays terminal chondrocyte differentiation. Matrix Biology 31:352-359. doi: 10.1016/j.matbio.2012.07.002
Symoens S, Malfait F, D′hondt S, Callewaert B, Dheedene A, Steyaert W, Bächinger H P, De Paepe A, Kayserili H, Coucke P J. 2013. Deficiency for the E R-stress transducer OASIS causes severe recessive osteogenesis imperfecta in humans. Orphanet J Rare Dis 8:154. doi: 10.1186/1750-1172-8-154
Tan Z, Niu B, Tsang K Y, Melhado I G, Ohba S, He X, Huang Y, Wang C, McMahon A P, Jauch R, Chan D, Zhang M Q, Cheah K S E. 2018. Synergistic co-regulation and competition by a SOX9-GLI-FOXA phasic transcriptional network coordinate chondrocyte differentiation transitions. PLOS Genet 14: e1007346. doi: 10.1371/journal.pgen.1007346
ten Berge D, Brouwer A, Korving J, Martin J F, Meijlink F. 1998. Prx1 and Prx2 in skeletogenesis: roles in the craniofacial region, inner ear and limbs. Development 125:3831-3842.
Tohmonda T, Miyauchi Y, Ghosh R, Yoda M, Uchikawa S, Takito J, Morioka H, Nakamura M, Iwawaki T, Chiba K, Toyama Y, Urano F, Horiuchi K. 2011. The IREla-XBP1 pathway is essential for osteoblast differentiation through promoting transcription of Osterix. EMBO Rep 12:451-457. doi: 10.1038/embor.2011.34
Tsang K Y, Tsang S W, Chan D, Cheah K S E. 2014. The chondrocytic journey in endochondral bone growth and skeletal dysplasia. Birth Defects Res C Embryo Today 102:52-73. doi: 10.1002/bdrc.21060
Tsao K-C, Lin Y-C, Chen Y-T, Lai S-L, Yang R-B. 2021. Zebrafish scube1 and scube2 cooperate in promoting Vegfa signaling during embryonic vascularization. Cardiovasc Res. doi: 10.1093/cvr/cvab125
Tsuda T, Markova D, Wang H, Evangelisti L, Pan T-C, Chu M-L. 2004. Zinc finger protein Zac1 is expressed in chondrogenic sites of the mouse. Dev Dyn 229:340-348. doi: 10.1002/dvdy.10439
Umeda K, Zhao J, Simmons P, Stanley E, Elefanty A, Nakayama N. 2012. Human chondrogenic paraxial mesoderm, directed specification and prospective isolation from pluripotent stem cells. Sci Rep 2:455. doi: 10.1038/srep00455
Varrault A, Gueydan C, Delalbre A, Bellmann A, Houssami S, Aknin C, Severac D, Chotard L, Kahli M, Digarcher A L, Pavlidis P, Journot L. 2006. Zac1 Regulates an Imprinted Gene Network Critically Involved in the Control of Embryonic Growth. Developmental Cell 11:711-722. doi: 10.1016/j.devcel.2006.09.003
Vishwanath N, Monis W J, Hoffmann G A, Ramachandran B, DiGiacomo V, Wong J Y, Smith M L, Layne M D. 2020. Mechanisms of aortic carboxypeptidase-like protein secretion and identification of an intracellularly retained variant associated with Ehlers-Danlos syndrome. J Biol Chem 295:9725-9735. doi: 10.1074/jbc.RA120.013902
Vlahos K, Sourris K, Mayberry R, McDonald P, Bruveris F F, Schiesser J V, Bozaoglu K, Lockhart P J, Stanley E G, Elefanty A G. 2019. Generation of iPSC lines from peripheral blood mononuclear cells from 5 healthy adults. Stem Cell Res 34:101380. doi: 10.1016/j.scr.2018.101380
Wagner E F. 2002. Functions of AP1 (Fos/Jun) in bone development. Annals of the Rheumatic Diseases 61: 40ii-4042. doi: 10.1136/ard.61.suppl_2.ii40
Wan M, Cao X. 2005. BMP signaling in skeletal development. Biochem Biophys Res Commun 328:651-657. doi: 10.1016/j.bbrc.2004.11.067
Wang L, Huang J, Moore D C, Zuo C, Wu Q, Xie L, von der Mark K, Yuan X, Chen D, Warman M L, Ehrlich M G, Yang W. 2017. SHP2 Regulates the Osteogenic Fate of Growth Plate Hypertrophic Chondrocytes. Sci Rep 7:12699. doi: 10.1038/s41598-017-12767-9
Wang W, Rigueur D, Lyons K M. 2014. TGFβ signaling in cartilage development and maintenance. Birth Defects Res C Embryo Today 102:37-51. doi: 10.1002/bdrc.21058
Wu C-L, Dicks A, Steward N, Tang R, Katz D B, Choi Y-R, Guilak F. 2021. Single cell transcriptomic analysis of human pluripotent stem cell chondrogenesis. Nat Commun 12:362. doi: 10.1038/s41467-020-20598-y
Xi H, Fujiwara W, Gonzalez K, Jan M, Liebscher S, Van Handel B, Schenke-Layland K, Pyle A D. 2017. In Vivo Human Somitogenesis Guides Somite Development from hPSCs. Cell Rep 18:1573-1585. doi: 10.1016/j.celrep.2017.01.040
Xing W, Godwin C, Pourteymoor S, Mohan S. 2019. Conditional disruption of the osterix gene in chondrocytes during early postnatal growth impairs secondary ossification in the mouse tibial epiphysis. Bone Res 7:24. doi: 10.1038/s41413-019-0064-9
Yang L, Tsang K Y, Tang H C, Chan D, Cheah K S E. 2014. Hypertrophic chondrocytes can become osteoblasts and osteocytes in endochondral bone formation. Proc Natl Acad Sci USA 111:12097-12102. doi: 10.1073/pnas.1302703111
Yip R K H, Chan D, Cheah K S E. 2019. Chapter Twelve-Mechanistic insights into skeletal development gained from genetic disorders In: Olsen B R, editor. Current Topics in Developmental Biology, Vertebrate Skeletal Development. Academic Press. pp. 343-385. doi: 10.1016/bs.ctdb.2019.02.002
Yu G, Wang L-G, Han Y, He Q-Y. 2012. clusterProfiler: an R package for comparing biological themes among gene clusters. OMICS 16:284-287. doi: 10.1089/omi.2011.0118
Zankl A, Duncan E L, Leo P J, Clark G R, Glazov E A, Addor M-C, Herlin T, Kim C A, Leheup B P, McGill J, McTaggart S, Mittas S, Mitchell A L, Mortier G R, Robertson S P, Schroeder M, Terhal P, Brown M A. 2012. Multicentric carpotarsal osteolysis is caused by mutations clustering in the amino-terminal transcriptional activation domain of MAFB. Am J Hum Genet 90:494-501. doi: 10.1016/j.ajhg.2012.01.003
Zhang Q, Cigan A D, Marrero L, Lopreore C, Liu S, Ge D, Savoie F H, You Z. 2011. Expression of doublecortin reveals articular chondrocyte lineage in mouse embryonic limbs. Genesis 49:75-82. doi: 10.1002/dvg.20702
Zhang Q, Zuo H, Yu S, Lin Y, Chen S, Liu H, Chen Z. 2020. RUNX2 co-operates with EGR1 to regulate osteogenic differentiation through Htral enhancers. J Cell Physiol 235:8601-8612. doi: 10.1002/jcp.29704
Zhou X, von der Mark K, Henry S, Norton W, Adams H, de Crombrugghe B. 2014. Chondrocytes transdifferentiate into osteoblasts in endochondral bone during development, postnatal growth and fracture healing in mice. PLOS Genet 10: e1004820. doi: 10.1371/journal.pgen.1004820

### CROSS REFERENCE TO RELATED APPLICATION

The present application claims priority to Australian provisional patent application No. 2021903310, which was filed on 14 Oct. 2022. The entire content of that application is hereby incorporated herein by reference.

1. A method for generating chondrocytes, chondrocyte-like cells, cartilage-like tissue, cartilage, or a combination thereof, the method comprising:
a) culturing a three-dimensional aggregation of sclerotome cells with an FGF pathway activator to produce a three-dimensional aggregation of chondrocyte progenitor cells; and
b) culturing the three-dimensional aggregation of chondrocyte progenitor cells generated in step a) in the absence of said FGF pathway activator to produce a three-dimensional aggregation of chondrocytes, or chondrocyte-like cells, that express COL2A1 and ACAN;
wherein the method comprises transferring the three-dimensional aggregation of sclerotome cells or the three-dimensional aggregation of chondrocyte progenitor cells to orbital culture.

2. The method of claim 1, wherein said sclerotome cells are obtained by a method comprising:
i) culturing a population of human pluripotent progenitor cells with a composition comprising a TGF-beta pathway activator, a Wnt pathway activator, an FGF pathway activator, and a P13K inhibitor for a time period of about 24 hours;
ii) culturing the cells of step i) with a composition comprising a TGF-beta pathway inhibitor, a Wnt pathway activator, an FGF pathway activator, and a BMP pathway inhibitor for a time period of about 24 hours;
iii) culturing the cells of step ii) with a composition comprising a Wnt pathway inhibitor, a BMP pathway inhibitor and a MEK/ERK pathway inhibitor for a time period of about 24 hours;
iv) contacting the cells of step iii) with a composition comprising a Wnt pathway inhibitor and a Hedgehog pathway activator, for a time period of about 72 hours to generate sclerotome cells.

3. The method of claim 1, wherein said sclerotome cells;
a) express any one of PAX1, SOX9, FOXC2, PAX9, NKX3.2/BAPX1 and TWIST1, and/or
b) are derived from a human embryonic stem cell population (hESC) or a human induced pluripotent stem cell population (PSC), and
optionally wherein said iPSCs are derived from a feeder-free cell culture.

4.-5. (canceled)

6. The method of claim 1, wherein step a) comprises transferring said aggregation of sclerotome cells to orbital culture at any time from day 7 of step a) onwards.

7. (canceled)

8. The method of claim 1, wherein said chondrocytes, chondrocyte-like cells, cartilage-like tissue, cartilage, or a combination thereof expresses:
i) one or more further genes selected from the group consisting of COL11A1, COL11A2, COL9A1, COLAK, COL9A3, MATN1 and MATN3; and/or
ii) collagen 2 (COL2A1) and ACAN at a ratio in the range of from 20:1 to 5:1, and/or
iii) does not express collagen COL1A1 or COL1A2 in levels greater than about 0.2% of the level of COL2A1.

9. The method of claim 1, wherein the chondrocytes, chondrocyte-like cells, cartilage-like tissue, cartilage, or a combination thereof express COL2A1 and ACAN at a ratio of about 10:1 and does not substantially express COL10A1

10. The method of claim 1, wherein the method further comprises culturing said three-dimensional aggregation of chondrocytes, or chondrocyte-like cells with triiodothyronine (T3) to produce a population of hypertrophic chondrocytes or hypertrophic chondrocyte-like cells, hypertrophic cartilage-like tissue, hypertrophic cartilage, or a combination thereof that express COL10A1 and/or expresses COL2A1 and COL10A1 at a ratio in the range of about 1:1 to about 2.5:1.

11.-13. (canceled)

14. The method of claim 1, wherein the method further comprises culturing said three-dimensional aggregation of chondrocytes, or chondrocyte-like cells with TGF-beta3, to produce a population of articular chondrocytes or articular chondrocyte-like cells, articular cartilage-like tissue, articular cartilage, or a combination thereof that express PRG4.

15. A method for generating articular chondrocytes or articular chondrocyte-like cells, articular cartilage-like tissue, articular cartilage, or a combination thereof, the method comprising:
a) culturing a three-dimensional aggregation of sclerotome cells with an FGF pathway activator to produce a three-dimensional aggregation of chondrocyte progenitor cells;
b) culturing the three-dimensional aggregation of chondrocyte progenitor cells with an FGF pathway activator and a TGF-beta agonist to produce a three-dimensional aggregation of chondrocytes, or chondrocyte-like cells, that express COL2A1 and ACAN; and
c) culturing said three-dimensional aggregation of chondrocytes, or chondrocyte-like cells with a TGF-beta agonist for an extended period of time to produce articular chondrocytes or articular chondrocyte-like cells, articular cartilage-like tissue, articular cartilage, or a combination thereof
wherein the method comprises transferring the three-dimensional aggregation of sclerotome cells, the three-dimensional aggregation of chondrocyte progenitor cells, or the three-dimensional aggregation of chondrocytes, or chondrocyte-like cells to orbital culture.

16. The method of claim 15, wherein said articular chondrocytes or articular chondrocyte-like cells, articular cartilage-like tissue, articular cartilage, or a combination thereof expresses COL2A1, ACAN, PRG4, and optionally, one or more further genes selected from the group consisting of ASPN, CILP and CILP2.

17. The method of claim 1, wherein said culturing in step a) and/or step b) is for a period of at least 7 days, from about 7-21 days, from about 10-17 days, about 14 days, or about 7 days.

18.-26. (canceled)

27. The method of claim 15, wherein said culturing in step c) is for a period of about 2 to about 5 weeks.

28. The method of claim 1, wherein the FGF pathway activator is selected from the group consisting of FGF2, FGF4, FGF9, FGF19, FGF21, FGF3, FGF5, FGF6, FGF8a, FGF16, FGF17, FGF18, FGF20 and FGF23, or is FGF2.

29.-45. (canceled)

46. The method of claim 10, further comprising:
culturing in orbital culture the hypertrophic chondrocytes, hypertrophic chondrocyte-like cells, hypertrophic cartilage-like tissue, hypertrophic cartilage with an osteogenic culture medium to generate osteoblasts or a bone-like organoid that express COL1A1 and COL1A2.

47. The method of claim 46 wherein said osteogenic differentiation culture medium comprises β-glycerophosphate, ascorbic acid 2-phosphate, sodium ascorbate, and dexamethasone, wherein said osteogenic differentiation culture medium comprises a WNT agonist for about the first 3-7 days of culture.

48. (canceled)

49. The method of claim 47, wherein said WNT agonist is CHIR99021.

50. The method of claim 1, further comprising a step of decellularizing said three-dimensional aggregation of chondrocytes, or chondrocyte-like cells, cartilage-like tissue or cartilage to produce a decellularized scaffold.

51. The method of claim 46, further comprising a step of decellularizing said bone-like organoid produce a decellularized scaffold; or degrading the extracellular matrix and isolating said osteoblasts.

52.-61. (canceled)

62. A method of treating a chondral defect or an osteochondral defect in a subject in need thereof comprising a) generating a composition comprising: chondrocytes, chondrocyte-like cells, cartilage-like tissue, cartilage, or a combination thereof produced according to claim 1.

63. A method of testing candidate chondrogenic or osteogenic modulating substances, the method comprising:
a) carrying out the method of claim 1, wherein said test substance is included in any one, or more, of the culture steps of the method;
b) assessing the effect of the test substance on chondrocyte or osteoblast proliferation, maintenance and/or differentiation compared to a control population generated in the absence of test substance; and
c) identifying the test substance as a candidate chondrogenic or osteogenic modulating substance if the test substance increases or decreases proliferation, and/or affects chondrocyte or osteoblast maintenance or differentiation compared to the control.

64. The method of claim 63, wherein the method is carried out using cells derived from a subject with a bone or cartilage disease or disorder, or cells engineered to have a mutation associated with a bone or cartilage disease or disorder or engineered to correct a mutation associated with a bone or cartilage disease or disorder.

65.-66. (canceled)

67. The method of claim 1 wherein the cells are derived from a subject with a bone or cartilage disease or disorder, or cells engineered to have a mutation associated with a bone or cartilage disease or disorder, or engineered to correct a mutation associated with a bone or cartilage disease or disorder.

68. (canceled)

69. A method of treating a chondral defect or an osteochondral defect in a subject in need thereof comprising a) generating a composition comprising: articular chondrocytes or articular chondrocyte-like cells, articular cartilage-like tissue, articular cartilage according to claim 15, and b) administering the composition to the subject.

70. A method of treating a chondral defect or an osteochondral defect in a subject in need thereof comprising a) generating a composition comprising: osteoblasts or bone-like organoid according to claim 46, and b) administering the composition to the subject.
